SEQUENCE LISTING (53 sequences)

SEQ ID NO: 1 - DNA, 23 bp, Artificial Sequence
  A synthesized primer for PCR
ggcacgtaagtaacacttcctgg

SEQ ID NO: 2 - DNA, 22 bp, Artificial Sequence
  A synthesized primer for PCR
gttacaggcacttacgggaacc

SEQ ID NO: 3 - DNA, 20 bp, Artificial Sequence
  A synthesized primer for PCR
ccagttgggcaatgcttatt

SEQ ID NO: 4 - DNA, 20 bp, Artificial Sequence
  A synthesized primer for PCR
ctcttgcttctcccaccttg

SEQ ID NO: 5 - DNA, 20 bp, Artificial Sequence
  An artificially synthesized primer for PCR
ttagctgtgctcgcgctact

SEQ ID NO: 6 - DNA, 20 bp, Artificial Sequence
  A synthesized primer for PCR
tcacatggttcacacggcag

SEQ ID NO: 7 - DNA, 21 bp, Artificial Sequence
  A synthesized primer for PCR
gaggtgatagcattgctttcg

SEQ ID NO: 8 - DNA, 21 bp, Artificial Sequence
  A synthesized primer for PCR
caagtcagtgtacaggtaagc

SEQ ID NO: 9 - DNA, 30 bp, Artificial Sequence
  A synthesized oligonucleotide for siRNA
aagcagtggtatcaacgcagagtacgcggg

SEQ ID NO: 10 - DNA, 45 bp, Artificial Sequence
  A synthesized primer for PCR
ctaatacgactcactatagggcaagcagtggtatcaacgcagagt

SEQ ID NO: 11 - DNA, 22 bp, Artificial Sequence
  A synthesized primer for PCR
ctaatacgactcactatagggc

SEQ ID NO: 12 - DNA, 26 bp, Artificial Sequence
  A synthesized primer for PCR
aactgcagacaggacatcagtcagca

SEQ ID NO: 13 - DNA, 23 bp, Artificial Sequence
  A synthesized primer for PCR
aagcagtggtatcaacgcagagt

SEQ ID NO: 14 - DNA, 26 bp, Artificial Sequence
  A synthesized primer for PCR
gcagacttcacagacatcaggtgtcc

SEQ ID NO: 15 - DNA, 23 bp, Artificial Sequence
  A synthesized primer for northern blot probe
ggcacgtaagtaacacttcctgg

SEQ ID NO: 16 - DNA, 22 bp, Artificial Sequence
  A synthesized primer for northern blot probe
gttacaggcacttacgggaacc

SEQ ID NO: 17 - DNA, 23 bp, Artificial Sequence
  A synthesized primer for northern blot probe
ggccattgattatcatctgaagc

SEQ ID NO: 18 - DNA, 22 bp, Artificial Sequence
  A synthesized primer for northern blot probe
tccttaccgtgtttgaaaggaa

SEQ ID NO: 19 - DNA, 19 bp, Artificial Sequence
  A synthesized target sequence for siRNA
gcgcgctttgtaggattcg

SEQ ID NO: 20 - DNA, 19 bp, Artificial Sequence
  A synthesized target sequence for siRNA
gcatgagagaagaccattc

SEQ ID NO: 21 - DNA, 19 bp, Artificial Sequence
  A synthesized target sequence for siRNA
ggacgtgcctttggaaatc

SEQ ID NO: 22 - DNA, 19 bp, Artificial Sequence
  A synthesized target sequence for siRNA
tcatgcgagcagaccattc

SEQ ID NO: 23 - DNA, 19 bp, Artificial Sequence
  A synthesized target sequence for siRNA
tcaacatgaggagacagtc

SEQ ID NO: 24 - DNA, 20 bp, Artificial Sequence
  A synthesized primer for PCR
ccagttgggcaatgcttatt

SEQ ID NO: 25 - DNA, 20 bp, Artificial Sequence
  A synthesized primer for PCR
ctcttgcttctcccaccttg

SEQ ID NO: 26 - DNA, 41 bp, Artificial Sequence
  A synthesized primer for PCR
atgcatgcctcgagttatgagagaagaccattcttttcatg

SEQ ID NO: 27 - DNA, 40 bp, Artificial Sequence
  A synthesized primer for PCR
acgtacgtgactcgagctaatggtctgccgattttttccc

SEQ ID NO: 28 - DNA, 84 bp, Artificial Sequence
  A synthesized primer for PCR
atgcatgcgctagcaagcatgtacccatacgatgttccagattacgctggaggaggaggaagagaagaccattcttttcatgtt

SEQ ID NO: 29 - DNA, 37 bp, Artificial Sequence
  An artificially synthesized primer for PCR
atgcatgcgatatcctaatggtctgccgattttttcc

SEQ ID NO: 30 - DNA, 39 bp, Artificial Sequence
  A synthesized primer for PCR
atgcatgccatatgagagaagaccattcttttcatgttc

SEQ ID NO: 31 - DNA, 40 bp, Artificial Sequence
  A synthesized primer for PCR
acgtacgtgactcgagatggtctgccgattttttccctga

SEQ ID NO: 32 - DNA, 33 bp, Artificial Sequence
  A synthesized primer for PCR
atgcatgcgaattcggcggatggaaggggccgg

SEQ ID NO: 33 - DNA, 35 bp, Artificial Sequence
  A synthesized primer for PCR
atgcatgcctcgagctactcagccaggctgttgcg

SEQ ID NO: 34 - DNA, 19 bp, Artificial Sequence
  A synthesized oligonucleotide for siRNA
gaagcagcacgacuucuuc

SEQ ID NO: 35 - DNA, 19 bp, Artificial Sequence
  A synthesized oligonucleotide for siRNA
gaagaagucgugcugcuuc

SEQ ID NO: 36 - DNA, 19 bp, Artificial Sequence
  A synthesized oligonucleotide for siRNA
ggacgugccuuuggaaauc

SEQ ID NO: 37 - DNA, 19 bp, Artificial Sequence
  A synthesized oligonucleotide for siRNA
gauuuccaaaggcacgucc

SEQ ID NO: 38 - DNA, 1855 bp, Homo sapiens
aattttatttaaaaattttttaaaagaatttggttttggattaaaaggcatgcaagcagcattaattccactcacagttacagtctatcacctggggcattcactacttttcagagtcagatcacagttcaaaagacagctctcaccttgggggcatattccccaagtcagtgtgaacatgtcttgtcccaagaggacactttatacgcatggatgttcacaaatttattctaaactgcaggtgtctgtcaggattcatttgttaaacatgtcctctgtgttaaaaatcctaaaatccttttgcatttcttagaaaaaatcacgtttctgatgaaattctaaagatgtttgcaatattaaactagaatatattaacatgtataattttgagacaaggtcttgctctgtcacccaggctggagtacagtggctcgatcataactcactgccacctcacgatcctcttgcctcagccctccgagtggctgggactgcaggcgtgtaccaccatgcctggctagttttatttttagtacatagccttgaacaactgagcttaagcgatcatcctccctcaaccttccaaagtgctgggactgtaggcatgagccactgcacctggcctgaaattacattgttacaggcacgtaagtaacacttcctggatccaaaaatgtgtattctgcatttcaacaactgaacttaagcgatcatcctccctcaaccttccaaagtgctaggactgtaggcatgagccactgcacctggcctaaaattacattgttacaggcacgtaagtaacacttcctggatccaaaaatgtgtattctgcatttcaacaactgaacttaagcgatcatcctccctcaaccttccaaagtgctaggactgtaggcatgagccactgcacctggcctaaaattacattgttacaggcacgtaagtaacacttcctggatccaaaaatgtgtattctgcatttcatattaaaataaaaaagcaagtgcttgggatacaaatatctgcatgaaaatttaaagactttattcctgactggtatcacttttagtaagcagttgaacataacttgtagtgtgaatatggctaaaacaaaggacacctgatgtctgtgaagtctgctaaggacaggtacaaaatttatgcattgctttttaaaaagtttaaaatgaggaatgcttttgataatcagaaagactaatgtaaagtgctgactgatgtcctgtctgcagttaaggaagacacccaactctcttcttcctcatcatggtattctctatgtatagatctctaaaaatgcaaacttcctatggacaagacaatatgatttgctataatatgaattaagatatggtaatatctaatagtctccactgctaggattctgagtaacacaaaaaataggttttataaaaagcccatgcacttcaattggtgggggaaaagaataaagtcattttcagtcgactgactctgtaaaacagattaccaatataacaagctatgttatctaaattgccctggcagtcttcagttttctctaagtaaacaaagtaaaaggaacaataaggatgcaggcgtagtaatagctgtggaaaagatgaaactacagccacagtgacctgtgggcagcctgcgtagagataagctgaggtgtcagtcagactgatcctggtaacgatactccatggctgcatgaagatgctggaggcatcaagtgggtggccttaactctagtggattcactaggcaatgtaaattggttagtgggttcccgtaagtgcctgtaacaataaaatttctttttcaatgtccccaaaaaaaaaaaaaaaaaaaa

SEQ ID NO: 39 - DNA, 5611 bp, Homo sapiens
aggcgcagaggagggcggtgttgagaccggcggagcggcgggacccctaggtggcggagggacgctccgggaaagcgaggggcgctacgagctctggcccacgtgacctgccgggggcgggagcagggggcgcgccggcctcctgcggtgcccctgccttggggaggggccgtgaccacccgtctgtcgcccgaggcggccgccgctgcaccttcaccgcgtacccgggacccgcccgcccgcgggagaaatgttgctgaagtgctgctgaaagggccagagatgcaaggatttgggatacattttgaacctttaagctgtctgacattgacctcctttcattattaataaagaagaatcaggagcttaggatgtattaacaccaactcattaatatactaaccggacaatgttctacaaacaattctacattgtaaaggactggattggcacaaaataaaataattttattttattcagcttataatatgactcgatggaggaaaatttgataagcatgagagaagaccattcttttcatgttcgttacagaatggaagcttcttgcctagagctggccttggaaggggaacgtctatgtaaatcaggagactgccgcgctggcgtgtcattctttgaagctgcagttcaagttggaactgaagacctaaaaacacttagcgctatttacagccagttgggcaatgcttatttctatttgcatgattatgccaaagcattagaatatcaccatcatgatttaacccttgcaaggactattggagaccagctgggggaagcgaaagctagtggtaatctgggaaacaccttaaaagttcttgggaattttgacgaagccatagtttgttgtcagcgacacctagatatttccagagagcttaatgacaaggtgggagaagcaagagcactttacaatcttgggaatgtgtatcatgccaaagggaaaagttttggttgccctggtccccaggatgtaggagaatttccagaagaagtgagagatgctctgcaggcagccgtggatttttatgaggaaaacctatcattagtgactgctttgggtgaccgagcggcacaaggacgtgcctttggaaatcttggaaacacacattacctccttggcaacttcagggatgcagttatagctcatgagcagcgtctccttattgcaaaagaatttggagataaagcagctgaaagaagagcatatagcaaccttggaaatgcatatatatttcttggtgaatttgaaactgcctcggaatactacaagaagacactactgttggcccgacagcttaaagaccgagctgtagaagcacagtcttgttacagtcttggaaatacatatactttacttcaagactatgaaaaggccattgattatcatctgaagcacttagcaattgctcaagagctgaatgatagaattggtgaaggaagagcatgttggagcttaggaaatgcatacacagcactaggaaatcatgatcaagcaatgcattttgctgaaaagcacttggaaatttcaagagaggttggggataaaagtggtgaactaacagcacgacttaatctctcagaccttcaaatggttcttggtctgagctacagcacaaataactccataatgtctgaaaatactgaaattgatagcagtttgaatggtgtacgccccaagttgggacgccggcatagtatggaaaatatggaacttatgaagttaacaccagaaaaggtacagaactggaacagtgaaattcttgctaagcaaaaacctcttattgccaaaccttctgcaaagctactctttgtcaacagactgaaggggaaaaaatacaaaacgaattcctccactaaagttctccaagatgccagtaattctattgaccaccgaattccaaattctcagaggaaaatcagtgcagatactattggagatgaagggttctttgacttattaagccgatttcaaagcaataggatggatgatcagagatgttgcttacaagaaaagaactgccatacagcttcaacaacaacttcttccactccccctaaaatgatgctaaaaacatcatctgttcctgtggtatcccccaacacggatgagtttttagatcttcttgccagctcacagagtcgccgtctggatgaccagagggctagtttcagtaatttgccagggcttcgtctaacacaaaacagccagtcggtacttagccacctgatgactaatgacaacaaagaggctgatgaagatttctttgacatccttgtaaaatgtcaaggatccagattagatgatcaaagatgtgctccaccacctgctaccacaaagggtccgacagtaccagatgaagactttttcagccttattttacggtcccagggaaagagaatggatgaacagagagttcttttacaaagagatcaaaacagagacactgactttgggctaaaggactttttgcaaaataatgctttgttggagtttaaaaattcagggaaaaaatcggcagaccattagttactatggatttattttttttcctttcaaacacgatattgaaggcagtttacaaggggatgataacaaagtaactaactaactgtagcaaaagacaagtatgggacagactgggacctggagtaacactggatcacagactgcaaaggcccacgcttggaacaagttgccaacttgtttcactctgcttgcttcagactgtgcacagttgggatgggatctggggatgtggacccctattctttcaaaaagtcattcaggtgatttggatgggcaaatagaactatttctctaatggccaatgttttttaagagtcataacctggaattaattacattaagtgctcagctaaaaaaaaaaaaaaaagttctaaattacaacctggattacattatgtttcatgtatactataaggtatgatgtcctggatagcattttaaaaactcaggtaagtttcatggggttcttataagaaaattcagttaagacaatataccaaaccacaacgtaaaaagtttgtgtatacaaactttttgtattatttgtacacaaagtacaaatttgtatttgtacacaaagtacaaactttgtgtattattttgtgtatacaatagttcttacagcctgtaaacattcaacattattccatttaaacattgtttttaacagctgtctaccctgttctgattttcctaaaagcttcacactatataggctgggcacagtggctcatgcctgtaatcccagcactttgggaggccgacaagggcgggtcacttgaggtcaggagttcgagaccagcctgaccaacatggtgaaaccccatctctactaaaaaaatacaaaaattagccaggcatggtgatgcacatctgtagtcctagctgctcgggagactgaggcaggagaatctcttgaacctgggaggtggaggttgcagtgagccgagattgtaccacctcactccagcctgggtgacagagcaaaactctcaaaaaaaagtaaaatttcaaaaaagcttcacaatataaaccttaattttaatgacatattggctagtcaataaacaagtcttatctcatctcatctcttttctgataacaaacacccgatgtgttcaatttgctttcatatttaagtctttcctgaattgctgtcatcattcaacaacagttgcatgtcgccttgctagctgtcaaagtagacttcatccccaaatggatatctgtaatgaaagaatacaaaggtgaaattttatttaaaaattttttaaaagaatttggttttggattaaaaggcatgcaagcagcattaattccactcacagttacagtctatcacctggggcattcactacttttcagagtcagatcacagttcaaaagacagctctcaccttgggggcatattccccaagtcagtgtgaacatgtcttgtcccaagaggacactttatacgcatggatgttcacaaatttattctaaactgcaggtgtctgtcaggattcatttgttaaacatgtcctctgtgttaaaaatcctaaaatccttttgcatttcttagaaaaaatcacgtttctgatgaaattctaaagatgtttgcaatattaaactagaatatattaacatgtataattttgagacaaggtcttgctctgtcacccaggctggagtacagtggctcgatcataactcactgccacctcacgatcctcttgcctcagccctccaagtagctgggactgcaggcgtgtaccaccatgcctggctagttttatttttagtacatagccttgaacaactgagcttaagcgatcatcctccctcaaccttccaaagtgctaggactgtaggcatgagccactgcacctggcctaaaattacattgttacaggcacgtaagtaacacttcctggatccaaaaatgtgtattctgcatttcaacaactgaacttaagcgatcatcctccctcgaccttccaaagtgctaggactgtaggcatgagccactgcacctggcctaaaattacattgttacaggcacgtaagtaacacttcctggatccaaaaatgtgtattctgcatttcaacaactgaacttaagcgatcatcctccctcaaccttccaaagtgctaggactgtaggcatgagccactgcacctggcctaaaattacattgttacaggcacgtaagtaacacttcctggatccaaaaatgtgtattctgcatttcatattaaaataaaaaagcaagtgcttgggatacaaatatctgcatgaaaatttaaagactttattcctgactggtatcacttttagtaagcagttgaacataacttgtagtgtgaatatggctaaaacaaaggacacctgatgtctgtgaagtctgctaaggacaggtacaaaatttatgcattgctttttaaaaagtttaaaatgaggaatgcttttgataatcagaaagactaatgtaaagtgctgactgatgtcctgtctgcagttaaggaagacacccaactctcttcttcctcatcatggtattctctatgtatagatctctaaaaatgcaaacttcctatggacaagacaatatgatttgctataatatgaattaagatatggtaatatctaatagtctccactgctaggattctgagtaacacaaaaaataggttttataaaaagcccatgcacttcaattggtgggggaaaagaataaagtcattttcagtcgactgactctgtaaaacagattaccaatataacaagctatgttatctaaattgccctggcagtcttcagttttctctaagtaaacaaagtaaaaggaacaataaggatgcaggcgtagtaatagctgtggaaaagatgaaactacagccacagtgacctgtgggcagcctgcgtagagataagctgaggtgtcagtcagactgatcctggtaacgatactccatggctgcatgaagatgctggaggcatcaagtgggtggccttaactctagtggattcactaggcaatgtaaattggttagtgggttcccgtaagtgcctgtaac

SEQ ID NO: 40 - protein, 684 aa, Homo sapiens
MEENLISMREDHSFHVRYRMEASCLELALEGERLCKSGDCRAGVSFFEAAVQVGTEDLKTLSAIYSQLGNAYFYLHDYAKALEYHHHDLTLARTIGDQLGEAKASGNLGNTLKVLGNFDEAIVCCQRHLDISRELNDKVGEARALYNLGNVYHAKGKSFGCPGPQDVGEFPEEVRDALQAAVDFYEENLSLVTALGDRAAQGRAFGNLGNTHYLLGNFRDAVIAHEQRLLIAKEFGDKAAERRAYSNLGNAYIFLGEFETASEYYKKTLLLARQLKDRAVEAQSCYSLGNTYTLLQDYEKAIDYHLKHLAIAQELNDRIGEGRACWSLGNAYTALGNHDQAMHFAEKHLEISREVGDKSGELTARLNLSDLQMVLGLSYSTNNSIMSENTEIDSSLNGVRPKLGRRHSMENMELMKLTPEKVQNWNSEILAKQKPLIAKPSAKLLFVNRLKGKKYKTNSSTKVLQDASNSIDHRIPNSQRKISADTIGDEGFFDLLSRFQSNRMDDQRCCLQEKNCHTASTTTSSTPPKMMLKTSSVPVVSPNTDEFLDLLASSQSRRLDDQRASFSNLPGLRLTQNSQSVLSHLMTNDNKEADEDFFDILVKCQGSRLDDQRCAPPPATTKGPTVPDEDFFSLILRSQGKRMDEQRVLLQRDQNRDTDFGLKDFLQNNALLEFKNSGKKSADH

SEQ ID NO: 41 - DNA, 3039 bp, Homo sapiens
aggcgcagaggagggcggtgttgagaccggcggagcggcgggacccctaggtggcggagggacgctccgggaaagcgaggggcgctacgagctctggcccacgtgacctgccgggggcgggagcagggggcgcgccggcctcctgcggtgcccctgccttggggaggggccgtgaccacccgtctgtcgcccgaggcggccgccgctgcaccttcaccgcgtacccgggacccgcccgcccgcgggagaaatgttgctgaagtgctgctgaaagggccagagatgcaaggatttgggatacattttgaacctttaagctgtctgacattgacctcctttcattattaataaagaagaatcaggagcttaggatgtattaacaccaactcattaatatactaaccggacaatgttctacaaacaattctacattgtaaaggactggattggcacaaaataaaataattttattttattcagcttataatatgactcgatggaggaaaatttgataagcatgagagaagaccattcttttcatgttcgttacagaatggaagcttcttgcctagagctggccttggaaggggaacgtctatgtaaatcaggagactgccgcgctggcgtgtcattctttgaagctgcagttcaagttggaactgaagacctaaaaacacttagcgctatttacagccagttgggcaatgcttatttctatttgcatgattatgccaaagcattagaatatcaccatcatgatttaacccttgcaaggactattggagaccagctgggggaagcgaaagctagtggtaatctgggaaacaccttaaaagttcttgggaattttgacgaagccatagtttgttgtcagcgacacctagatatttccagagagcttaatgacaaggtgggagaagcaagagcactttacaatcttgggaatgtgtatcatgccaaagggaaaagttttggttgccctggtccccaggatgtaggagaatttccagaagaagtgagagatgctctgcaggcagccgtggatttttatgaggaaaacctatcattagtgactgctttgggtgaccgagcggcacaaggacgtgcctttggaaatcttggaaacacacattacctccttggcaacttcagggatgcagttatagctcatgagcagcgtctccttattgcaaaagaatttggagataaagcagctgaaagaagagcatatagcaaccttggaaatgcatatatatttcttggtgaatttgaaactgcctcggaatactacaagaagacactactgttggcccgacagcttaaagaccgagctgtagaagcacagtcttgttacagtcttggaaatacatatactttacttcaagactatgaaaaggccattgattatcatctgaagcacttagcaattgctcaagagctgaatgatagaattggtgaaggaagagcatgttggagcttaggaaatgcatacacagcactaggaaatcatgatcaagcaatgcattttgctgaaaagcacttggaaatttcaagagaggttggggataaaagtggtgaactaacagcacgacttaatctctcagaccttcaaatggttcttggtctgagctacagcacaaataactccataatgtctgaaaatactgaaattgatagcagtttgaatggtgtacgccccaagttgggacgccggcatagtatggaaaatatggaacttatgaagttaacaccagaaaaggtacagaactggaacagtgaaattcttgctaagcaaaaacctcttattgccaaaccttctgcaaagctactctttgtcaacagactgaaggggaaaaaatacaaaacgaattcctccactaaagttctccaagatgccagtaattctattgaccaccgaattccaaattctcagaggaaaatcagtgcagatactattggagatgaagggttctttgacttattaagccgatttcaaagcaataggatggatgatcagagatgttgcttacaagaaaagaactgccatacagcttcaacaacaacttcttccactccccctaaaatgatgctaaaaacatcatctgttcctgtggtatcccccaacacggatgagtttttagatcttcttgccagctcacagagtcgccgtctggatgaccagagggctagtttcagtaatttgccagggcttcgtctaacacaaaacagccagtcggtacttagccacctgatgactaatgacaacaaagaggctgatgaagatttctttgacatccttgtaaaatgtcaaggatccagattagatgatcaaagatgtgctccaccacctgctaccacaaagggtccgacagtaccagatgaagactttttcagccttattttacggtcccagggaaagagaatggatgaacagagagttcttttacaaagagatcaaaacagagacactgactttgggctaaaggactttttgcaaaataatgctttgttggagtttaaaaattcagggaaaaaatcggcagaccattagttactatggatttattttttttcctttcaaacacggtaaggaaacaatctattacttttttccttaaaaggagaatttatagcactgtaatacagcttaaaatatttttagaatgatgtaaatagttaaccttcagtagtctattaaggcattaatacttctctggacatgcgcgtttgagggtggaggggtcctgtaaggtgcttcatcgtctgtgattactgcttgggatgtgttctttggcagcttgtgagattactttacctagtgtttataaagtaggaagttaagtgaatcatagattagaatttaatactcttatggaaataattttttaacatcttaattgacaatggcgtttttttatacataaccatggatgtagtgggaaacaatgttgtttggtaaaaataatgtacttgatcaatgtaaaaaagtatataaaatagtcttactaaaaatctaggtttttttttcctccaaaaaaa

SEQ ID NO: 42 - DNA, 10024 bp, Homo sapiens
cccaggggaggaggtgaaattcctcagctctccaccaagattggccacaaaagcctgatcccctggaacacagcaggcctcacatagacggtcagccattggatcataggaactgccctggcctgactcacccaatatggaggaggtgcctggggatgccctgtgtgaacactttgaggccaacatacttacccagaaccgctgtcaaaactgcttccaccctgaggaggcccatggagcaagataccaggagctcaggagcccttcaggtgctgaggtgccctactgcgacctgcctcgatgtccacctgcccctgaggacccactcagcgcctcaacctccggctgccagtctgtggtggacccaggcctcaggccagggcccaagaggggcccatccccctcagcagggctcccagaagagggtcccacagctgcccccaggagcaggagccgggagcttgaggcagtaccctatctggagggcctgaccacttccttgtgtggcagctgcaacgaggaccccggctctgaccccacctccagccctgactccgccacccctgatgataccagcaactcgtcctctgtggactgggacactgttgagaggcaggaggaggaggcccccagctgggacgagctcgcagtgatgatcccgaggaggcctcgggaggggccgagagctgacagctcccaaagggctccgtctctcctcaccaggtcccctgtgggaggagatgctgcaggccagaaaaaggaggacaccggcggtgggggccggagcgcaggacagcactgggcaaggctccggggagaaagcgggttgtccctggagcggcaccggtcaacactgacccaggcttcctccatgacaccacacagtggacctcgaagcaccacgtctcaggcttctcctgcccaaagggacactgctcaggctgcctctacacgtgaaatccccagagcctcctctccccatcgaatcacccaaagggacacctccagggcctcatccacccaacaggaaatctccagggcctcatccacccaacaggaaacctccagggcctcatccacccaagaggacacccctagggcctcatccacccaagaggacacccccagggcctcatctacacagtggaacacccccagagcttcctctccctcacgaagcacccaactggataaccccagaacctcttctacccagcaggacaacccccaaacttcttttcctacttgtactccccagcgggaaaaccccaggacaccctgtgtccagcaggacgatcccagagcctcctctcccaacagaaccactcaacgagagaattccagaacatcctgtgcccagcgggacaatcccaaagcctccagaacctcctctcccaatagagccacacgagacaaccccagaacatcctgcgcccagcgggacaatcccagagcctcctctcccagtagagctacacgagacaaccccacaacatcctgtgcccagcgggacaatcccagagcctccagaacctcctctcccaatagagccacacgagacaaccccagaacatcctgtgcccagcgggacaatcccagagcctcctctcccagtagagctacacgagacaaccccacaacatcctgtgcccagcgggacaatcccagagcctccagaacctcctctcccaatagagccacacgagacaaccccagaacatcctgcgcccagcgggacaatcccagagcctcctctcccaatagagctgcacgagacaaccccacaacatcctgtgcccagcgggacaatcccagagcctccagaacctcctctcccaatagagccacacgagacaaccccagaacatcctgtgcccagcgggacaatcccagagcctcctctcccaatagagctacacgagacaaccccacaacatcctgtgcccagcgggacaatcccagagcctccagaacctcctctcccaatagagccacacgagataaccccagaacatcctgtgcccagcgggacaatcccagagcctcctctcccaacagaaccacccaacaagacagccccagaacatcctgtgcccgacgggacgatcccagagcctcctctcctaacagaaccatccaacaagagaaccccagaacatcctgtgccctacgggacaatcccagagcctcctctcccagcagaaccatccaacaagagaaccccagaacatcctgtgcccaacgggacgatcccagagcctcctctcctaacagaaccacccaacaagagaaccccagaacatcctgtgcccgacgggacaatcccagagcctcctctcgcaacagaaccatccagcgagacaaccccagaacatcctgtgcccagcgggacaatcccagagcctcctctcctaacagaaccatccaacaagagaacctcagaacatcctgtacccgacaggacaatcccaggacctcctctcccaatagagccacacgagacaaccccagaacatcctgtgcccagcgggacaatctcagagcctcctctcccatcagagccacccaacaggacaaccccagaacttgtattcaacagaacatccccagatcatcttctacccaacaagacaaccctaaaacctcttgtaccaaacgagataacctcagacccacttgtacacagcgggaccgcacacagtccttttcctttcaacgagacaaccctggaacctcctcatctcaatgctgcacccaaaaggagaatctgagaccatcatctccccaccgctccactcaatggaacaatcccaggaattcatctccccatcgtactaacaaagacatcccctgggcctcgtttcccctccggccaactcagagtgatggtccccgaacctcttccccatctcgctccaagcaaagcgaggttccctgggcatccatcgccctccggccaacccaaggtgacaggcctcagacatcctctcccagcaggccagcccagcatgacccaccccagtcctcctttggccccacccagtacaacttgccatcccgggccacctcttcctcccataacccaggccaccagagcacctcccgaacttcctcacctgtgtaccccgctgcctatggggctcccctgacctctcctgagccctcccagcctccatgtgctgtgtgcattgggcaccgggatgcccctcgagcctcttcgccccctcgctatttgcagcacgaccccttccccttcttcccagagccccgcgcccctgagagtgaaccgccccaccacgagcctccctatataccacctgctgtgtgcattggacaccgagatgccccccgggcgtcctcgcccccccgccacacccaatttgaccccttccccttcctcccagacacatcagatgccgagcatcagtgtcagtccccccaacacgagccccttcagctccctgcacctgtgtgtattgggtaccgagatgcaccccgggcctcctccccaccacgccaggccccagagccttccctcttattccaggacctccccagggccagcacagagagccttgtcccttccatggactctctgcacgagtgcccccacatccccacccctgtgtgcattgggcaccgggatgcaccctccttctcatccccaccacgccaggctcctgagccatccctcttcttccaggatccccctggaactagtatggagagcctggccccctccactgactctctgcatggctccccagtgctgatcccccaagtgtgcatcgggcaccgggatgcaccccgagcctcctccccaccccgccacccacccagtgacctagcgttcctggcaccctcaccttcaccgggcagctctgggggctcccggggctcagcgcctcccggggagaccaggcacaacttggagcgggaggagtacactgtgctggccgacctgcccccacccaggaggctggcccagagacagccagggccccaggcgcagtgcagcagcgggggccgcacccacagccctggccgtgcagaggtggagcgcctcttcgggcaagagcgcaggaagtccgaggcagcgggggccttccaggcccaggacgagggacggtcacagcagcccagccaaggccagagccaacttctccgaagacagtccagccctgcccccagcaggcaggtgaccatgctccctgccaaacaggcagaactgacccggcggagccaagcagagccccctcatccttggagtcctgagaagagacctgagggagatcggcagctccaggggtccccgctgccccccaggacatcagccaggacccctgagagggagctgcggacacagagacctctggagagtggccaagcaggcccaagacagcctctgggggtgtggcagagtcaggaggaaccgccagggtcccagggccctcatagacacctagaaaggagctggagcagccaggagggaggcctgggccctgggggctggtggggatgtggagagcccagcctgggggcagccaaagccccggagggagcatgggggggcacttccagggagtacaaggagagctgggggcagccagaggcctgggaggagaagcccactcatgagctccccagagaactaggaaagagaagcccactcacgagcccccctgagaactggggaggccccgcagagtcctcacaatcctggcactctgggacacccactgctgtgggctggggggcagagggagcgtgtccatacccgcgtggctctgagaggcgacccgagcttgactggagggatctgcttggccttctccgggcaccaggagagggggtctgggcccgtgtccccagcctggactgggagggcctcttggagctcctgcaggccaggctgccccgcaaggacccagctggacacagggatgacctggccagggctttagggccagagctgggtcccccaggcacaaacgatgtccctgagcaggagtcacacagccagccagaaggctgggccgaggccaccccagtcaatggacacagccccgcactgcagtcccagagcccggtccagctgcccagccctgcctgcacctccacccagtggccaaagatcaaagtgacaagaggaccagcgaccgcaactctggcaggcctggagcagacgggccccctggggagcaggagcactgcgaagggccccagcttgccagagctgcagttccaaccagaggagcctgaggagtcagaaccaagcagaggccaagaccccctgactgaccagaagcaggcagactcggcagacaagaggccagcagagggcaaggctgggagcccgctcaagggccgactggtgacctcatggcggatgcccggggaccggcccacgctgttcaatccgttcctgctgtctctgggggtcctcaggtggcgaaggcccgatctgctcaacttcaagaagggatggatgtcgatcttggacgagcctggagagcctccctccccctcgctcaccaccacctctacttcgcagtggaagaaacattggtttgtgctgacagattcaagtctcaaatattacagagactccactgctgaggaggcagatgagctggatggtgagatcgacctgcgttcctgcacggatgtcactgagtacgcggtgcagcgcaactatggcttccagatccacaccaaggatgctgtctataccttgtcggccatgacctcaggcatccggcggaactggatcgaggctctgagaaagaccgtacgtccaacttcagccccagatgtcaccaagctctcggactctaacaaggagaacgcgctgcacagctacagcacccagaagggccccctgaaggcaggggagcagcgggcgggctctgaggtcatcagccggggtggccctcggaaggcggacgggcagcgtcaggccttggactacgtggagctctcgccgctgacccaggcttccccgcagcgggcccgcaccccagcccgcactcctgaccgcctggccaagcaggaggagctggagcgggacctggcccagcgctccgaggagcggcgcaagtggtttgaggccacagacagcaggaccccagaggtgcctgctggtgaggggccgcgccggggcctgggtgcccccctgactgaggaccagcaaaaccggcttagtgaggagatcgagaagaagtggcaggagctggagaagctgcccctgcgggagaataagcgggtgcccctcactgccctgctcaaccaaagccgcggagagcgccgagggcccccaagtgacggccacgaggcactggagaaggaggttcaggctcttcgggcccagctggaggcgtggcgtctccaaggggaggctcctcagagtgcactgagatcccaggaggatggccacatccccccgggctacatctcacaggaggcatgtgagcgcagcctggcagagatggagtcctcgcaccagcaggtgatggaggagctgcagcggcaccacgagcgggagctgcagcgcctgcagcaggagaaggagtggctcctggctgaggagacggcagccacggcctcagccattgaagccatgaagaaggcctaccaggaagagctgagccgagagctgagcaaaacacggagtctccagcagggcccggatggcctccggaagcagcaccagtcagatgtggaggcactgaagcgagagctgcaggtgctatcggagcagtactcgcagaagtgcctggagattggggcactcatgcggcaggctgaggagcgcgagcacacgctgcgccgctgccagcaggagggccaggagctgctgcgccacaaccaggagctgcatggccgcctgtcagaggagatagaccagctgcgcggcttcattgcctcgcagggcatgggcaatggctgcgggcgcagcaacgagcggagttcctgcgagctagaggtgctgcttcgcgtaaaagaaaacgaactccagtacctaaagaaggaggtgcagtgcctccgggacgagctccagatgatgcagaaggacaagcgcttcacctcgggaaagtaccaggacgtctatgtggagctgagccacatcaagacacggtctgagcgggagatcgagcagctgaaggagcacctgcgtcttgccatggccgccctccaggagaaggagtcgatgcgcaacagcctggctgagtagaggtcccgcccagctgcagaccctccaggctggaggaccagccgccctccttccctcctggatggaagtaaaaagccaagctttctccccaccctctgtgggccacacgtgcacttgcacccaccacacacacacacacacacacacacacacacacagacacacagacacatacgcacacacgtgcacacatgtacacacggatacacacacacacacacacactgcatatctgagcgcgcccctcgcactgggtctcaccttgcaccttcttcaggattttatatgtgaagagatttttatatagatttttttcctttttttccaaaacactttatactttaaaaaaaaaaaaaaaaagcaattcctggtggctgtgtgcctccaaccctggtccccctctgtctccagccaccctctgcttgggcttctgagctggtggccctggcccagaggtctggcggaggcccaggcagcagccatggcggggtgtctctacaggggagaggcgggagcctgccaccctcttcctgccctacctcctactaacacttcctgccccatttggacccgtaccatggggctcaggacagagggagctagcagctggcctccatggccccacagcctccttcgaggctgtgctgggtgcagaaccgccagagccacccaaaaggtgtttctcttctgctccctgaacctcttaacttaataaaacgttccagcagctctggtgtcctagatggctggcagaacaggaatggagattggggtttcttgaagtggcttccccactcacaccctacccacaccccacccttcctaaagcagcggcctctaggcttctgagggtggggctgaaaatccaaggtctcccttagtacagactgtgacgcccccagtgtggcttgcaggctagtggcagcggaagcatgtgggaaatagcaagtccagtcccaccccaacctactgaaccagcgtctgcattttaacagggtgccccagtgattcacgtgaccttcaaagtctgaggagccctggagtagggcccagggcccaggcctgagagagaggcctggggctaagactagccctgaccatgaccttgggcaagtcacacccctcactgagcctcagtttgcttctccatcaaatgggtgtttttctttcagtcccagtcctctcctgaggcttttcccagaggtcatgcccagccctgggcaggggtgagggcagggttgtggctgccaggcacaggtccagcgggctgggagtcctcaccatggctggctgtgctgattatggttgcatggggctgggaagaggtcccatttctgagccacagaaacaggccacatccagtaggggtcctggaaggctgtgagaacccagaggagaccctgacccagcctgcaggtagaagactgcctttggaggcagctttgaaagatgactcttccagatggaagaatcaggaagggccttcccacgtggaggcaccagcaggagcaaacgtccctggtgtgttcaggcaaagagccacaggagagtgacctcgtggagtgggaagagattgtttcccattcccttgccagggattggttgaggaatggacaaggcttgcagttcacgcaagccagtgaaatgcgaggggaagtcttgggagtggggtggaggaggtcatctgggaagggtgtccttggtcttaaaaagaggcacaagaggccgggcgcggtggctcttgcctattatcctagcactttgggaggccaaggcagggggatcacttgaggtcaggagttcgagaccagcctggccaacatggtgaaaccccgtctctactaaaaatacaaaaaattagccagacgtggtgccaagcacctgtaatcccagctactcaggaggctgaggcaggagaatcacttgaacccaggaggcagaagttgcagtgagccaagatcgcgccactgcactccagcctgtgtgacaagggcgaaactccatctcaaaaaaaaagggggagggtggcgcaaggagaggacacctctctcctggccctgtcgccatcttccctcatcaatagctaaaaatggcaaagaagaaagagccaggaccccgtgccatcaaatcctccggccttgtgctgccctgtctcggcactgcttgccttaggagatcaggagtgttcctagccactgggagatgggatgcctgccactggcagctctgccgtccatcctcaccatcccaccgtccccccaagacagctctccatcccatcctctgcctcccttctagcctcaacctaactcagggctcccccgtctcacacctgggttgcaggaacatcctcttcaccaacccccctcccatccctcctcagggctgcctagggcagctacaaaaaccaggccagcctctgaaaggtgctccacatctcattaaaaaccgtgaaatcgcactgccccactatcagaatagctcagaggagaaggtctgccagtaccaagtgctggtggggatacggagaagagcagctcccaggcgctgctggtgcaaatgtgctttgttgcacccacggcaggaaactgaccagcttctcatcaagttaaatacgcatctaccctatggcctagcatttccactcttgcttacacatccaggagaaatgagtccttacatgcacaaaaggatgagtaccgtaatactcacatcagcaaataaacaatacagctgtgtgtcagtagtagaatgaataaattgtggtcgattcataaaaaaaaaaaaaaa

SEQ ID NO: 43 - protein, 2365 aa, Homo sapiens
MEEVPGDALCEHFEANILTQNRCQNCFHPEEAHGARYQELRSPSGAEVPYCDLPRCPPAPEDPLSASTSGCQSVVDPGLRPGPKRGPSPSAGLPEEGPTAAPRSRSRELEAVPYLEGLTTSLCGSCNEDPGSDPTSSPDSATPDDTSNSSSVDWDTVERQEEEAPSWDELAVMIPRRPREGPRADSSQRAPSLLTRSPVGGDAAGQKKEDTGGGGRSAGQHWARLRGESGLSLERHRSTLTQASSMTPHSGPRSTTSQASPAQRDTAQAASTREIPRASSPHRITQRDTSRASSTQQEISRASSTQQETSRASSTQEDTPRASSTQEDTPRASSTQWNTPRASSPSRSTQLDNPRTSSTQQDNPQTSFPTCTPQRENPRTPCVQQDDPRASSPNRTTQRENSRTSCAQRDNPKASRTSSPNRATRDNPRTSCAQRDNPRASSPSRATRDNPTTSCAQRDNPRASRTSSPNRATRDNPRTSCAQRDNPRASSPSRATRDNPTTSCAQRDNPRASRTSSPNRATRDNPRTSCAQRDNPRASSPNRAARDNPTTSCAQRDNPRASRTSSPNRATRDNPRTSCAQRDNPRASSPNRATRDNPTTSCAQRDNPRASRTSSPNRATRDNPRTSCAQRDNPRASSPNRTTQQDSPRTSCARRDDPRASSPNRTIQQENPRTSCALRDNPRASSPSRTIQQENPRTSCAQRDDPRASSPNRTTQQENPRTSCARRDNPRASSRNRTIQRDNPRTSCAQRDNPRASSPNRTIQQENLRTSCTRQDNPRTSSPNRATRDNPRTSCAQRDNLRASSPIRATQQDNPRTCIQQNIPRSSSTQQDNPKTSCTKRDNLRPTCTQRDRTQSFSFQRDNPGTSSSQCCTQKENLRPSSPHRSTQWNNPRNSSPHRTNKDIPWASFPLRPTQSDGPRTSSPSRSKQSEVPWASIALRPTQGDRPQTSSPSRPAQHDPPQSSFGPTQYNLPSRATSSSHNPGHQSTSRTSSPVYPAAYGAPLTSPEPSQPPCAVCIGHRDAPRASSPPRYLQHDPFPFFPEPRAPESEPPHHEPPYIPPAVCIGHRDAPRASSPPRHTQFDPFPFLPDTSDAEHQCQSPQHEPLQLPAPVCIGYRDAPRASSPPRQAPEPSLLFQDLPRASTESLVPSMDSLHECPHIPTPVCIGHRDAPSFSSPPRQAPEPSLFFQDPPGTSMESLAPSTDSLHGSPVLIPQVCIGHRDAPRASSPPRHPPSDLAFLAPSPSPGSSGGSRGSAPPGETRHNLEREEYTVLADLPPPRRLAQRQPGPQAQCSSGGRTHSPGRAEVERLFGQERRKSEAAGAFQAQDEGRSQQPSQGQSQLLRRQSSPAPSRQVTMLPAKQAELTRRSQAEPPHPWSPEKRPEGDRQLQGSPLPPRTSARTPERELRTQRPLESGQAGPRQPLGVWQSQEEPPGSQGPHRHLERSWSSQEGGLGPGGWWGCGEPSLGAAKAPEGAWGGTSREYKESWGQPEAWEEKPTHELPRELGKRSPLTSPPENWGGPAESSQSWHSGTPTAVGWGAEGACPYPRGSERRPELDWRDLLGLLRAPGEGVWARVPSLDWEGLLELLQARLPRKDPAGHRDDLARALGPELGPPGTNDVPEQESHSQPEGWAEATPVNGHSPALQSQSPVQLPSPACTSTQWPKIKVTRGPATATLAGLEQTGPLGSRSTAKGPSLPELQFQPEEPEESEPSRGQDPLTDQKQADSADKRPAEGKAGSPLKGRLVTSWRMPGDRPTLFNPFLLSLGVLRWRRPDLLNFKKGWMSILDEPGEPPSPSLTTTSTSQWKKHWFVLTDSSLKYYRDSTAEEADELDGEIDLRSCTDVTEYAVQRNYGFQIHTKDAVYTLSAMTSGIRRNWIEALRKTVRPTSAPDVTKLSDSNKENALHSYSTQKGPLKAGEQRAGSEVISRGGPRKADGQRQALDYVELSPLTQASPQRARTPARTPDRLAKQEELERDLAQRSEERRKWFEATDSRTPEVPAGEGPRRGLGAPLTEDQQNRLSEEIEKKWQELEKLPLRENKRVPLTALLNQSRGERRGPPSDGHEALEKEVQALRAQLEAWRLQGEAPQSALRSQEDGHIPPGYISQEACERSLAEMESSHQQVMEELQRHHERELQRLQQEKEWLLAEETAATASAIEAMKKAYQEELSRELSKTRSLQQGPDGLRKQHQSDVEALKRELQVLSEQYSQKCLEIGALMRQAEEREHTLRRCQQEGQELLRHNQELHGRLSEEIDQLRGFIASQGMGNGCGRSNERSSCELEVLLRVKENELQYLKKEVQCLRDELQMMQKDKRFTSGKYQDVYVELSHIKTRSEREIEQLKEHLRLAMAALQEKESMRNSLAE

SEQ ID NO: 44 - DNA, 1840 bp, Homo sapiens
ggagggttcgaattgcaacggcagctaccgggcgtatgtgttggtgctagaggcagctgcagggtctcgctgggggccgctcgggaccaattttgaagaggtacttggccacgacttattttcacctccgacctttccttccaggcggtgagactctggactgagagtggctttcacaatggaagggatcagtaatttcaagacaccaagcaaattatcagaaaaaaagaaatctgtattatgttcaactccaactataaatatcccggcctctccgattatgcagaagcttggctttggtactggggtaaatgtgtacctaatgaaaagatctccaagaggtttgtctcattctccttgggctgtaaaaaagattaatcctatatgtaatgatcattatcgaagtgtgtatcaaaagagactaatggatgaagctaagattttgaaaagccttcatcatccaaacattgttggttatcgtgcttttactgaagccagtgatggcagtctgtgtcttgctatggaatatggaggtgaaaagtctctaaatgacttaatagaagaacgatataaagccagccaagatccttttccagcagccataattttaaaagttgctttgaatatggcaagagggttaaagtatctgcaccaagaaaagaaactgcttcatggagacataaagtcttcaaatgttgtaattaaaggcgattttgaaacaattaaaatctgtgatgtaggagtctctctaccactggatgaaaatatgactgtgactgaccctgaggcttgttacattggcacagagccatggaaacccaaagaagctgtggaggagaatggtgttattactgacaaggcagacatatttgcctttggccttactttgtgggaaatgatgactttatcgattccacacattaatctttcaaatgatgatgatgatgaagataaaacttttgatgaaagtgattttgatgatgaagcatactatgcagccttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcgtaaataactgtttattccaaaatatttacatagttactatcagtagttattagactctaaaattggcatatttcaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaccattaaattgaatgccattacttttattaatgatctttcttaaatattctatattttaatggatctactgacattagcactttgtacagtacaaaataaagtctacatttgtttaaaacactgaaccttttgctgatgtgtttatcaaatgataactggaagctgaggagaatatgcctcaaaaagagtagctccttggatacttcagactctggttacagattgtcttgatctcttggatctcctcagatcttctttggtttttgctttaatttattaaatgtattttccatactgagtttaaaatttattaatttgtaccttaagcatttcccagctgtgtaaaaacaataaaactcaaataggatgataaagaataaaggacactttgggtaaaaaaaaaaaaaa

SEQ ID NO: 45 - protein, 322 aa, Homo sapiens [sequence omitted]

SEQ ID NO: 46 - DNA, 25 bp, Artificial Sequence
  A synthesized primer for PCR
cgccggcatgctatggaaaatatgg

SEQ ID NO: 47 - DNA, 25 bp, Artificial Sequence
  A synthesized primer for PCR
ccatattttccatagcatgccggcg

SEQ ID NO: 48 - DNA, 24 bp, Artificial Sequence
  A synthesized primer for PCR
acttcttccgctccccctaaaatg

SEQ ID NO: 49 - DNA, 24 bp, Artificial Sequence
  A synthesized primer for PCR
cattttagggggagcggaagaagt

SEQ ID NO: 50 - DNA, 24 bp, Artificial Sequence
  A synthesized primer for PCR
cagagggctgctttcagtaatttg

SEQ ID NO: 51 - DNA, 24 bp, Artificial Sequence
  A synthesized primer for PCR
caaattactgaaagcagccctctg

SEQ ID NO: 52 - DNA, 2336 bp, Homo sapiens
  CDS (174)..(2207)
ggcacgaggaagaatcaggagcttaggatgtattaacaccaactcattaatatactaaccggacaatgttctacaaacaattctacattgtaaaggactggattggcacaaaataaaataattttattttattcagcttataatatgactcgatggaggaaaatttgataagcatgagagaagaccattcttttcatgttcgttacagaatggaagcttcttgcctagagctggccttggaaggggaacgtctatgtaaatcaggagactgccgcgctggcgtgtcattctttgaagctgcagttcaagttggaactgaagacctaaaaacacttagcgctatttacagccagttgggcaatgcttatttctatttgcatgattatgccaaagcattagaatatcaccatcatgatttaacccttgcaaggactattggagaccagctgggggaagcgaaagctagtggtaatctgggaaacaccttaaaagttcttgggaattttgacgaagccatagtttgttgtcagcgacacctagatatttccagagagcttaatgacaaggtgggagaagcaagagcactttacaatcttgggaatgtgtatcatgccaaagggaaaagttttggttgccctggtccccaggatgtaggagaatttccagaagaagtgagagatgctctgcaggcagccgtggatttttatgaggaaaacctatcattagtgactgctttgggtgaccgagcggcacaaggacgtgcctttggaaatcttggaaacacacattacctccttggcaacttcagggatgcagttatagctcatgagcagcgtctccttattgcaaaagaatttggagataaagcagctgaaagaagagcatatagcaaccttggaaatgcatatatatttcttggtgaatttgaaactgcctcggaatactacaagaagacactactgttggcccgacagcttaaagaccgagctgtagaagcacagtcttgttacagtcttggaaatacatatactttacttcaagactatgaaaaggccattgattatcatctgaagcacttagcaattgctcaagagctgaatgatagaattggtgaaggaagagcatgttggagcttaggaaatgcatacacagcactaggaaatcatgatcaagcaatgcattttgctgaaaagcacttggaaatttcaagagaggttggggataaaagtggtgaactaacagcacgacttaatctctcagaccttcaaatggttcttggtctgagctacagcacaaataactccataatgtctgaaaatactgaaattgatagcagtttgaatggtgtactccccaagttgggacgccggcatagtatggaaaatatggaacttatgaagttaacaccagaaaaggtacagaactggaacagtgaaattcttgctaagcaaaaacctcttattgccaaaccttctgcaaagctactctttgtcaacagactgaaggggaaaaaatacaaaacgaattcctccactaaagttctccaagatgccagtaattctattgaccaccgaattccaaattctcagaggaaaatcagtgcagatactattggagatgaagggttctttgacttattaagccgatttcaaagcaataggatggatgatcagagatgttgcttacaagaaaagaactgccatacagcttcaacaacaacttcttccactccccctaaaatgatgctaaaaacatcatctgttcctgtggtatcccccaacacggatgagtttttagatcttcttgccagctcacagagtcgccgtctggatgaccagagggctagtttcagtaatttgccagggcttcgtctaacacaaaacagccagtcggtacttagccacctgatgactaatgacaacaaagaggctgatgaagatttctttgacatccttgtaaaatgtcaaggatccagattagatgatcaaagatgtgctccaccacctgctaccacaaagggtccgacagtaccagatgaagactttttcagccttattttacggtcccagggaaagagaatggatgaacagagagttcttttacaaagagatcaaaacagagacactgactttgggctaaaggactttttgcaaaataatgctttgttggagtttaaaaattcagggaaaaaatcggcagaccattagttactatggatttattttttttcctttcaaacacggtaaggaaacaatctattacttttttccttaaaaggagaatttatagcactgtaatacagcttaaaatatttttagaatgatgtaaatagttaa

SEQ ID NO: 53 - protein, 677 aa, Homo sapiens
MREDHSFHVRYRMEASCLELALEGERLCKSGDCRAGVSFFEAAVQVGTEDLKTLSAIYSQLGNAYFYLHDYAKALEYHHHDLTLARTIGDQLGEAKASGNLGNTLKVLGNFDEAIVCCQRHLDISRELNDKVGEARALYNLGNVYHAKGKSFGCPGPQDVGEFPEEVRDALQAAVDFYEENLSLVTALGDRAAQGRAFGNLGNTHYLLGNFRDAVIAHEQRLLIAKEFGDKAAERRAYSNLGNAYIFLGEFETASEYYKKTLLLARQLKDRAVEAQSCYSLGNTYTLLQDYEKAIDYHLKHLAIAQELNDRIGEGRACWSLGNAYTALGNHDQAMHFAEKHLEISREVGDKSGELTARLNLSDLQMVLGLSYSTNNSIMSENTEIDSSLNGVLPKLGRRHSMENMELMKLTPEKVQNWNSEILAKQKPLIAKPSAKLLFVNRLKGKKYKTNSSTKVLQDASNSIDHRIPNSQRKISADTIGDEGFFDLLSRFQSNRMDDQRCCLQEKNCHTASTTTSSTPPKMMLKTSSVPVVSPNTDEFLDLLASSQSRRLDDQRASFSNLPGLRLTQNSQSVLSHLMTNDNKEADEDFFDILVKCQGSRLDDQRCAPPPATTKGPTVPDEDFFSLILRSQGKRMDEQRVLLQRDQNRDTDFGLKDFLQNNALLEFKNSGKKSADH

(19) United States
(12) Patent Application Publication
(10) Pub. No.: US 2011/0313018 A1
(43) Pub. Date: Dec. 22, 2011

(54) CANCER RELATED GENE, LGN/GPSM2

(75) Inventors: Yusuke Nakamura, Tokyo (JP); Toyomasa Katagiri, Tokyo (JP); Chikako Fukukawa, Kanagawa (JP)

(73) Assignee: Oncotherapy Science, Inc., Kanagawa (JP)

(21) Appl. No.: 13/061,123

(22) PCT Filed: Aug. 21, 2009
(86) PCT No.: PCT/JP2009/004017

§ 371 Date: May 19, 2011

Related U.S. Application Data
(60) Provisional application No. 61/190,395, filed on Aug. 27, 2008.

(51) Int. Cl. A61K 31/713; A61P 35/00; C12N 15/85; C07H 21/04; C12Q 1/68; C12N 15/113

(52) U.S. Cl. 514/44 A; 435/6.14; 435/6.12; 435/7.1; 435/6.13; 536/24.5; 435/320.1; 536/23.5

(57) ABSTRACT

The present invention provides methods for detecting and diagnosing cancer, which method involves the determination of the expression level of the LGN/GPSM2 gene. Furthermore, the present invention provides methods of screening for therapeutic agents useful in the treatment or prevention of cancer and methods for treating breast cancer. Moreover, the present invention provides siRNAs targeting the LGN/GPSM2 gene, which are useful in the treatment or prevention of cancer.

### PRIORITY

This application claims the benefit of U.S. Provisional Application No. 61/190,395, filed on Aug. 27, 2008, the entire disclosure of which is hereby incorporated herein by reference.

The present invention relates to methods for detecting and diagnosing cancer as well as methods for treating and preventing cancer.

### TECHNICAL FIELD

### BACKGROUND ART

Breast cancer is the most common cancer in women, with estimated new cases of 1.15 million worldwide in 2002 (NPL 1:Parkin D M et al., 2005 CA Cancer J Clin 55:74-108). Incidence rates of breast cancer are increasing in most countries, and the increasing rate is much higher in countries where its incidence was previously low (NPL 1:Parkin D M et al., 2005 CA Cancer J Clin 55:74-108). Early detection with mammography as well as development of molecular targeted drugs such as tamoxifen and trastuzumab have reduced the mortality rate and made the quality of life of patients better (NPL 2:Navolanic and McCubrey, Int J Oncol. 2005 27:1341-1344, NPL 3:Bange et al., 2001 Nat Med. 7:548-552). However, still very limited treatment options are available to patients at an advanced stage, particularly those with a hormone-independent tumors. Hence, development of novel drugs to provide better management to such patients is still needed.

Gene-expression profiles obtained by cDNA microarray analysis have provided detailed characterization of individual cancers and such information should contribute to choose more appropriate clinical strategies to individual patients through development of novel drugs and providing the basis of personalized treatment (NPL 4:Petricoin et al., Nat Genet. 2002 32 Suppl:474-479). Through the genome-wide expression analysis, the present inventors have isolated a number of genes that function as oncogenes contributing to the development and/or progression of breast cancers (NPL 5:Park J H et al., Cancer Res. 2006 66:9186-9195, NPL 6:Shimo et al., Cancer Sci. 2007 98:174-181, NPL 7:Lin M L et al., Breast Cancer Res. 2007 9, R17), synovial sarcomas (NPL 8:Nagayama S, et al. (2004) Oncogene 23:5551-5557., NPL 9:Nagayama S, et al. (2005) Oncogene 24:6201-6212.;), and renal cell carcinomas (NPL 10: Togashi et al., Cancer Res. 2005 65:4817-4826, NPL 11:Hirota et al., Int J Oncol. 2006 29:799-827). Such molecules are considered to be candidate targets for development of new therapeutic modalities.

In an attempt to identify novel molecular targets for breast cancer therapy, the inventors previously analyzed the detailed gene-expression profiles of breast cancer cells, which were purified by laser microbeam microdissection, by means of cDNA microarray (NPL 12:Nishidate et al., Int J Oncol. 2004 25:797-819). The present invention is based, in part, on the elucidation of the pathophysiologic role in brease cancer of a LGN/GPSM2 (Leu-Gly-Asn repeat-enriched protein/G-protein signalling modulator 2) gene that was previously isolated as a protein that interact with the alpha-subunit of the heterotrimeric GTP-binding protein, Gi2 (NPL 13:Mochizuki et al., Gene. 1996 181:39-43) by yeast two hybrid system.

### CITATION LIST

### Non Patent Literature

[NPL 1] Parkin D M et al., 2005 CA Cancer J Clin 55:74-108

[NPL 2] Navolanic and McCubrey, Int J Oncol. 2005 27:1341-1344,

[NPL 3] Bange et al., 2001 Nat Med. 7:548-552

[NPL 4] Petricoin et al., Nat Genet. 2002 32 Suppl:474-479

[NPL 5] Park J H et al., Cancer Res. 2006 66:9186-9195

[NPL 6] Shimo et al., Cancer Sci. 2007 98:174-181

[NPL 7] Lin M L et al., Breast Cancer Res. 2007 9:R17

[NPL 8] Nagayama S, et al. (2004) Oncogene 23:5551-5557

[NPL 9] Nagayama S, et al. (2005) Oncogene 24:6201-6212.

[NPL 10] Togashi et al., Cancer Res. 2005 65:4817-4826

[NPL 11] Hirota et al., Int J Oncol. 2006 29:799-827

[NPL 12] Nishidate et al., Int J Oncol. 2004 25:797-819

[NPL 13] Mochizuki et al., Gene.1996 181:39-43

### SUMMARY OF INVENTION

The present invention is based, in part, on the discovery of a specific expression pattern of the LGN/GPSM2 gene in cancerous cells.

Through the present invention, the LGN/GPSM2 gene was revealed to be frequently up-regulated in human tumors, in particular, breast cancer. Moreover, since the suppression of the LGN/GPSM2 gene by small interfering RNA (siRNA) resulted in growth inhibition and/or cell death of cancer cells, this gene serves as a therapeutic target for human cancers.

The LGN/GPSM2 gene identified herein as well as its transcription and translation products find diagnostic utility as a marker for cancer and as an oncogene target, the expression and/or activity of which may be altered to treat or alleviate symptoms of breast cancer. Similarly, by detecting the changes in the expression of the LGN/GPSM2 gene due to a compound, various compounds can be identified as agents for treating or preventing cancer.

Accordingly, the present invention provides methods for diagnosing or determining a predisposition to cancer in a subject by determining the expression level of the LGN/GPSM2 gene in a subject-derived biological sample, for example, a tissue sample. An increased expression level of the LGN/GPSM2 gene in the tissue or cells of the biological sample as compared to the expression level of LGN/GPSM2 in the tissue or cells of a normal control indicates that the subject suffers from or is at risk of developing cancer. The normal control level can be determined using a normal cell obtained from a non-cancerous tissue, for example, normal breast tissue.

In the context of the present invention, the phrase “control level” refers to the expression level of the LGN/GPSM2 gene detected in a control sample and includes both normal control level and cancer control level. A control level can be a single expression pattern derived from a single reference population or the average calculated from a plurality of expression patterns. Alternatively, the control level can be a database of expression patterns from previously tested cells. A “normal control level” refers to a level of the LGN/GPSM2 gene expression detected in a normal healthy individual or in a population of individuals known not to be suffering from cancer. A normal individual is one with no clinical symptom of cancer. A “normal control level” may also be the expression level of the LGN/GPSM2 gene detected in the normal healthy tissue or cell of an individual or population known not to be suffering from breast cancer. On the other hand, a “cancer control level” refers to an expression level of the LGN/GPSM2 gene detected in a cancerous tissue or cell of an individual or population suffering from breast cancer.

An increase in the expression level of the LGN/GPSM2 gene detected in a sample as compared to a normal control level indicates that the subject (from which the sample has been obtained) suffers from or is at risk of developing cancer.

Alternatively, expression level of the LGN/GPSM2 gene in a sample can be compared to cancer control level of the LGN/GPSM2 gene. A similarity between the expression level of a sample and the cancer control level indicates that the subject (from which the sample has been obtained) suffers from or is at risk of developing cancer.

Herein, gene expression levels are deemed to be “increased” when the gene expression increases by, for example, 10%, 25%, or 50% from, or at least 0.1 fold, at least 0.2 fold, at least 0.5 fold, at least 2 fold, at least 5 fold, or at least 10 fold or more in a test sample compared to a normal control level. The expression level of the LGN/GPSM2 gene can be determined by detecting using any method known in the art, including without limitation, e.g., hybridization intensity of nucleic acid probes to and/or quantitative amplification of gene transcripts in a sample.

In the context of the present invention, subject-derived tissue samples may be any breast tissues obtained from test subjects, e.g., patients known to have or suspected of having breast cancer. For example, tissues may comprise breast epithelial cells. More particularly, tissues may be cancerous breast epithelial cells.

The present invention further provides methods for screening a candidate compound for treating or preventing cancer using the LGN/GPSM2 polypeptide, the LGN/GPSM2 polynucleotide, the transcriptional regulatory region thereof, or a cell expressing LGN/GPSM2.

The present invention also provides a kit that comprises at least one detection reagent which binds to the transcription or translation product of the LGN/GPSM2 gene.

The present invention includes methods for treating or preventing cancer in a subject, comprising the step of administering to a subject a double-stranded molecule or vector encoding thereof, wherein the double-stranded molecule inhibits the expression of the LGN/GPSM2 gene.

The present invention further provides a composition for treating or preventing cancer, comprising a double-stranded molecule or a vector encoding thereof, wherein the double-stranded molecule inhibits the expression of the LGN/GPSM2 gene.

The present invention further provides a double-stranded molecule against the LGN/GPSM2 gene or a vector encoding thereof, wherein the double stranded molecule inhibits the cancer cell growth, as well as the expression of the LGN/GPSM2 gene.

Other features and advantages of the present invention will be apparent from the following detailed description, and from the claims.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

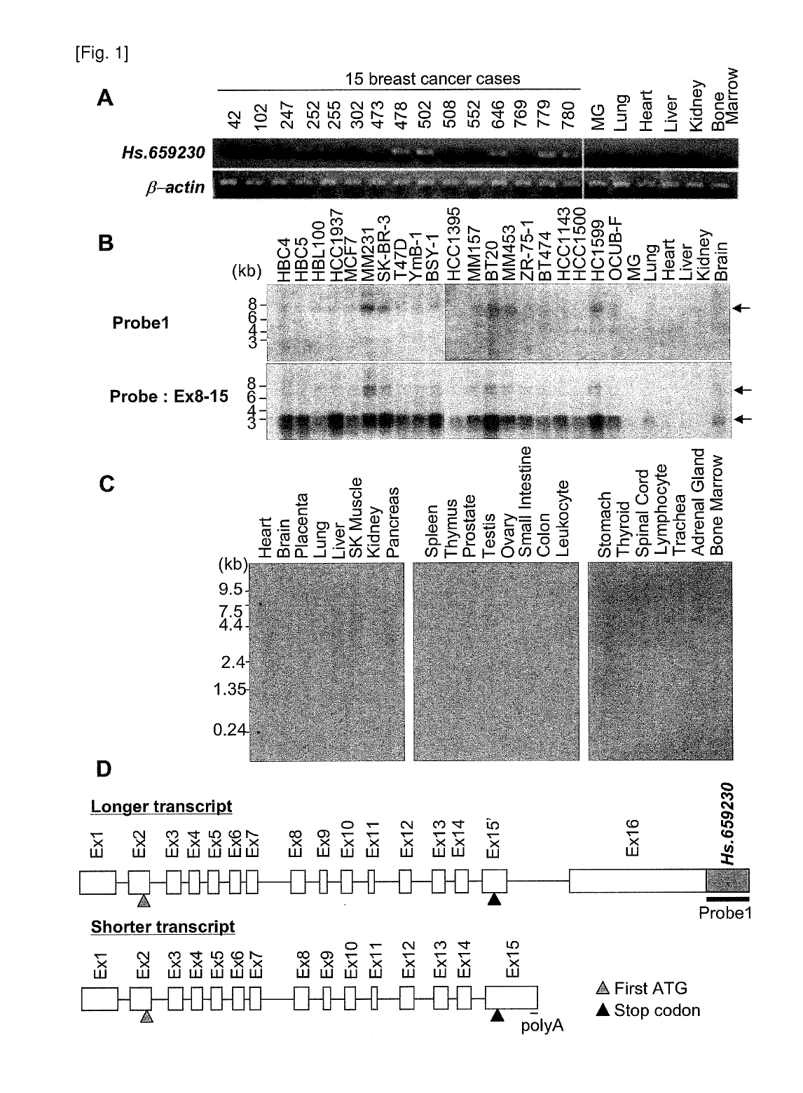

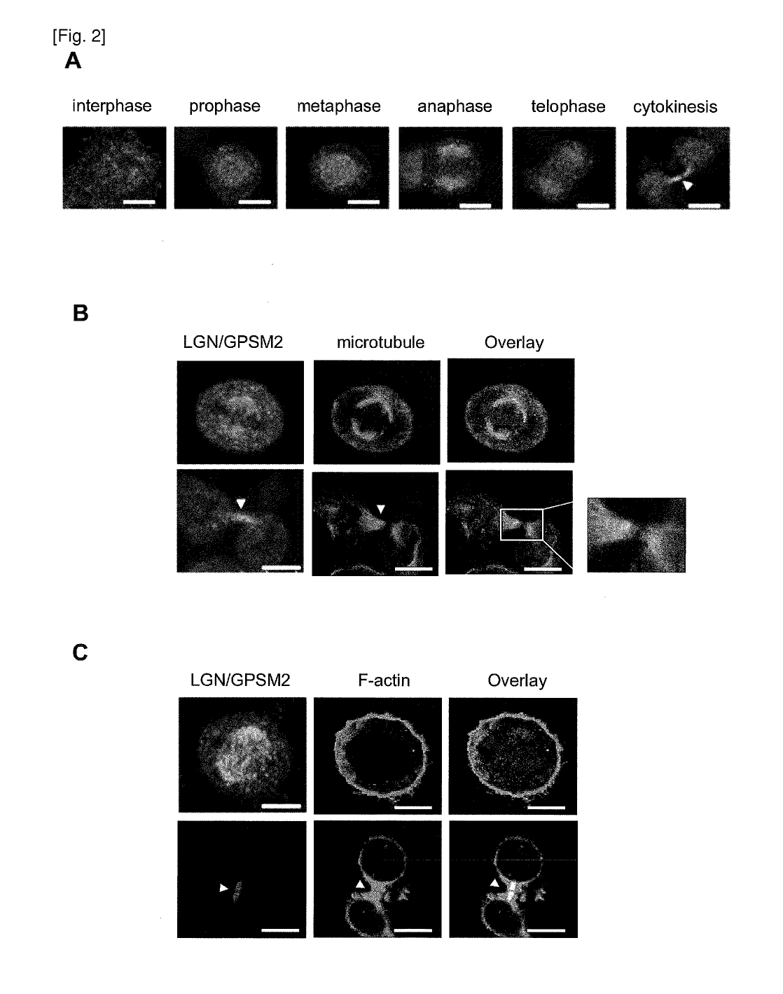

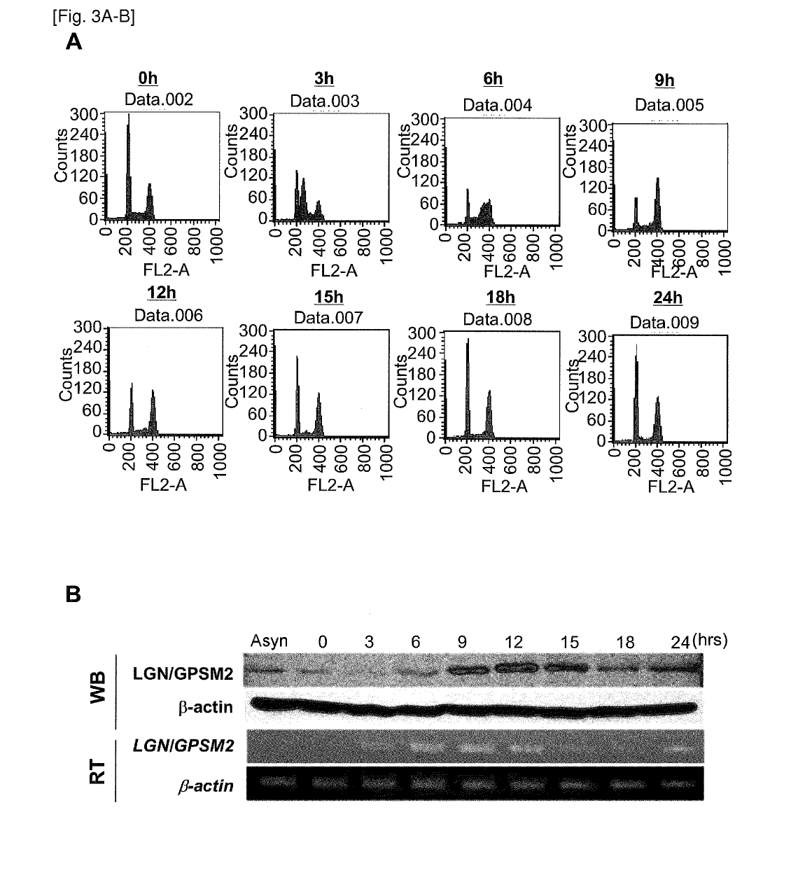

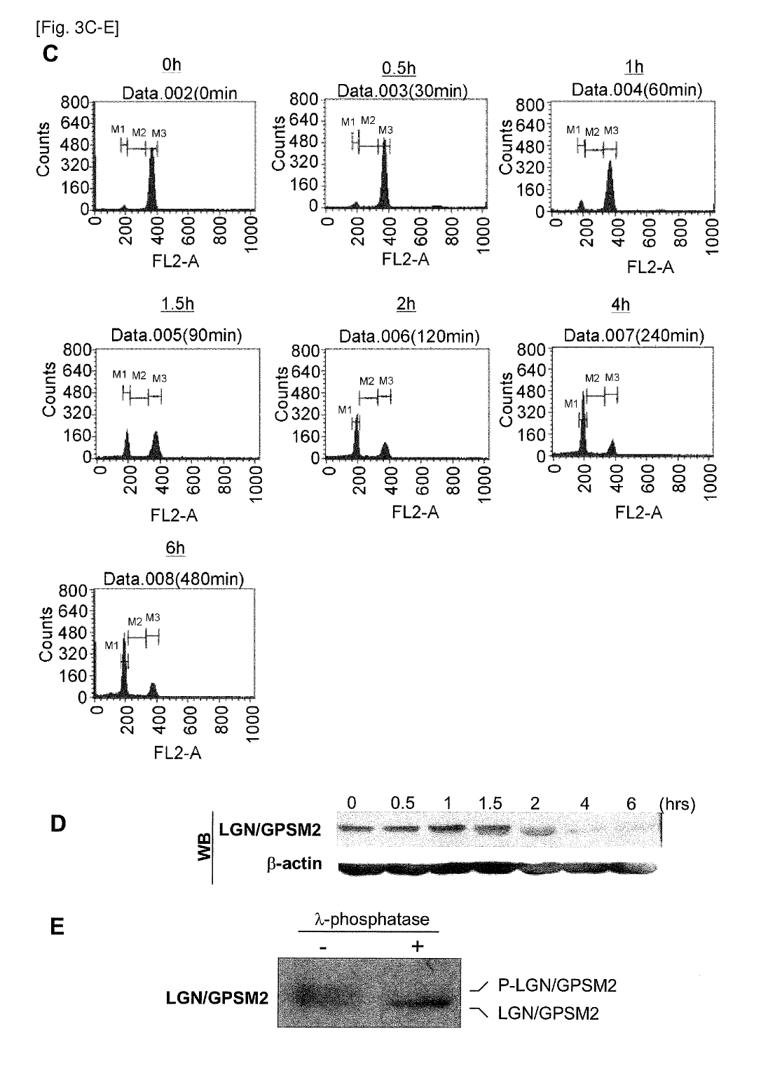

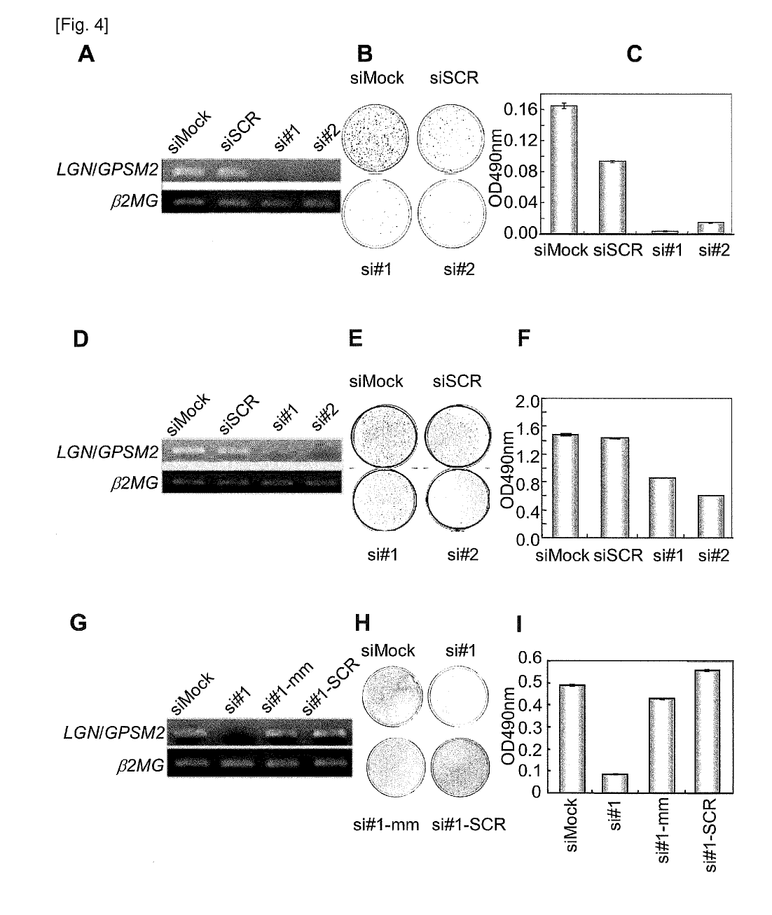

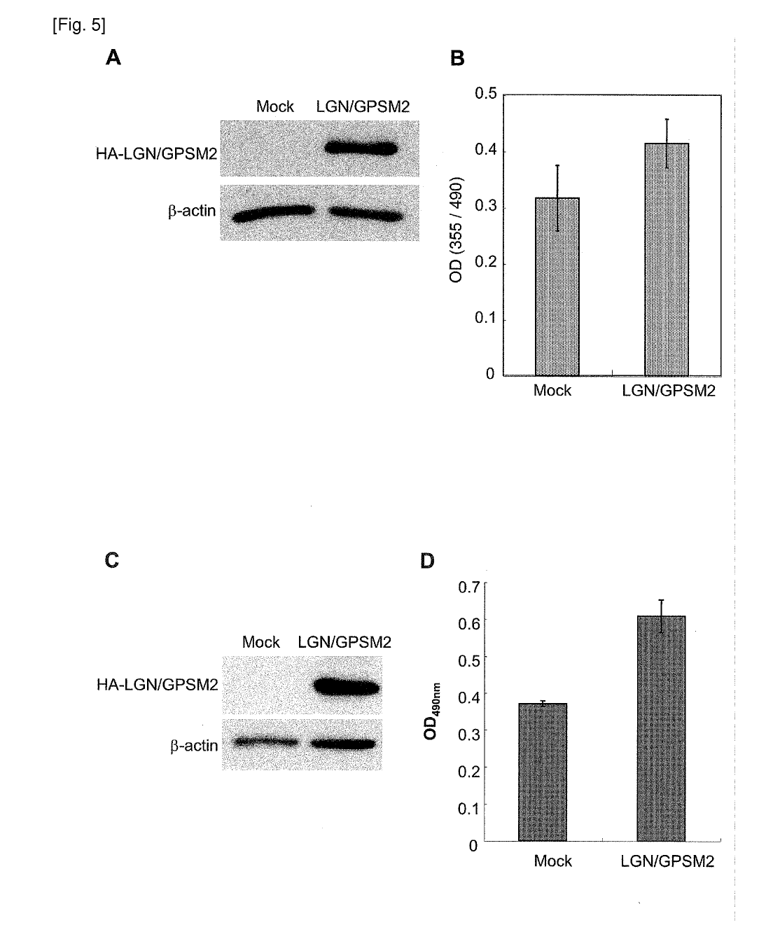

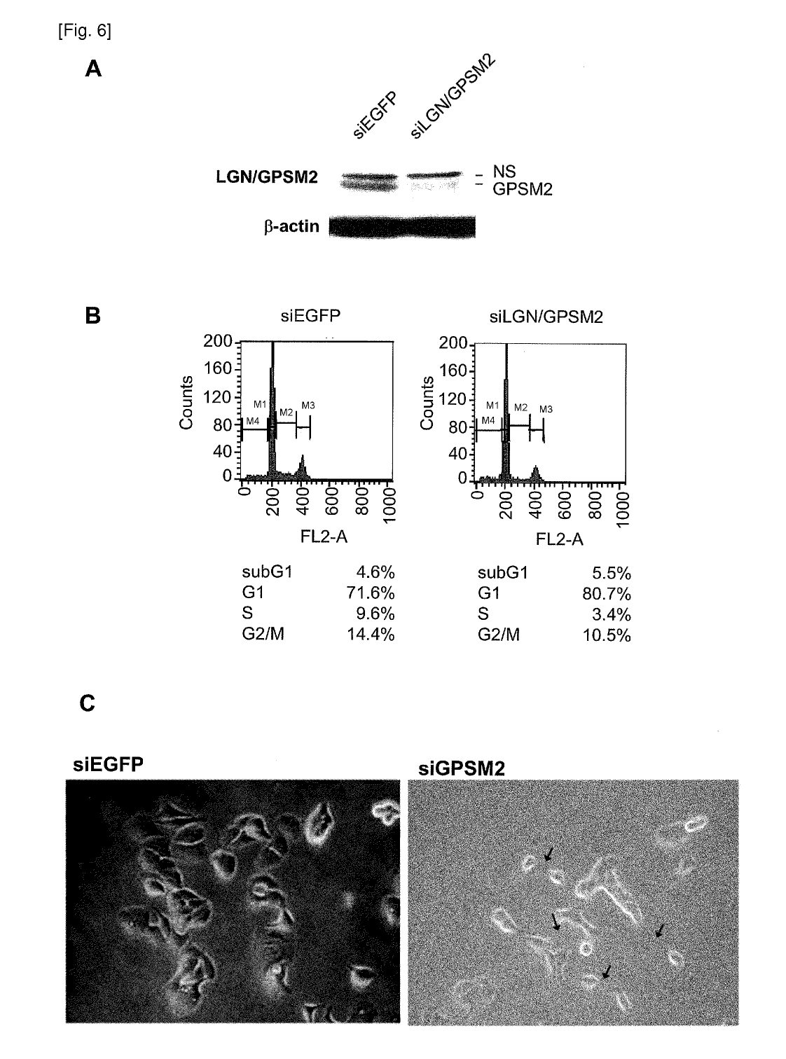

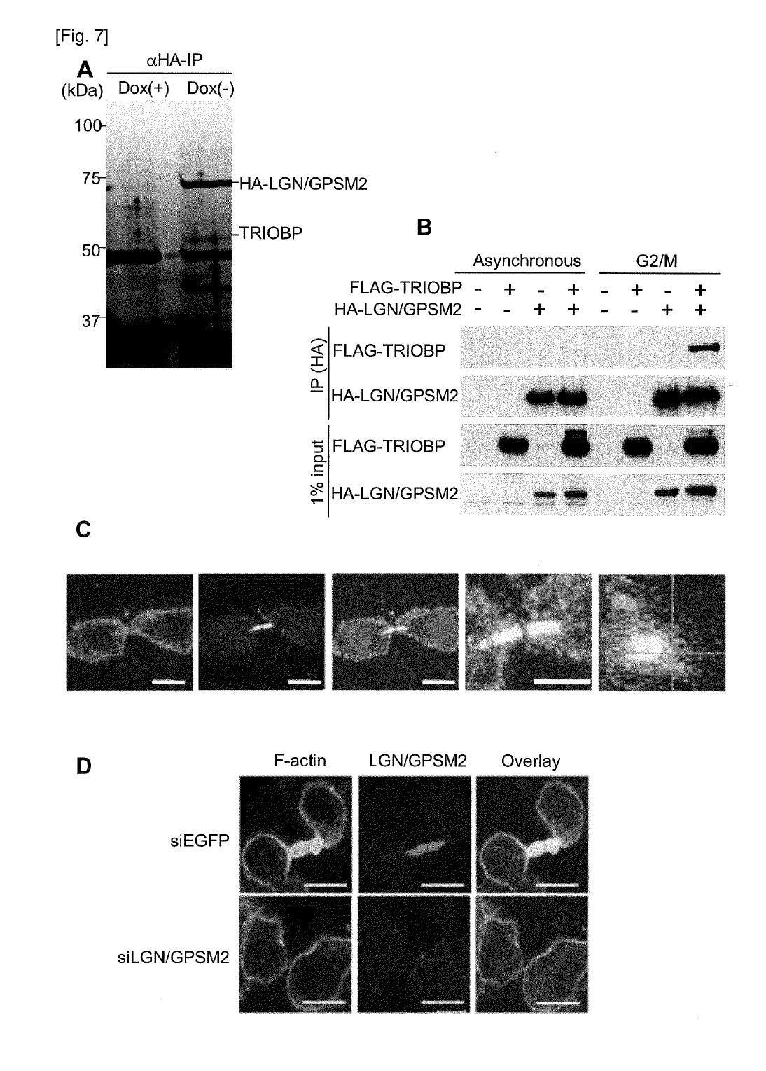

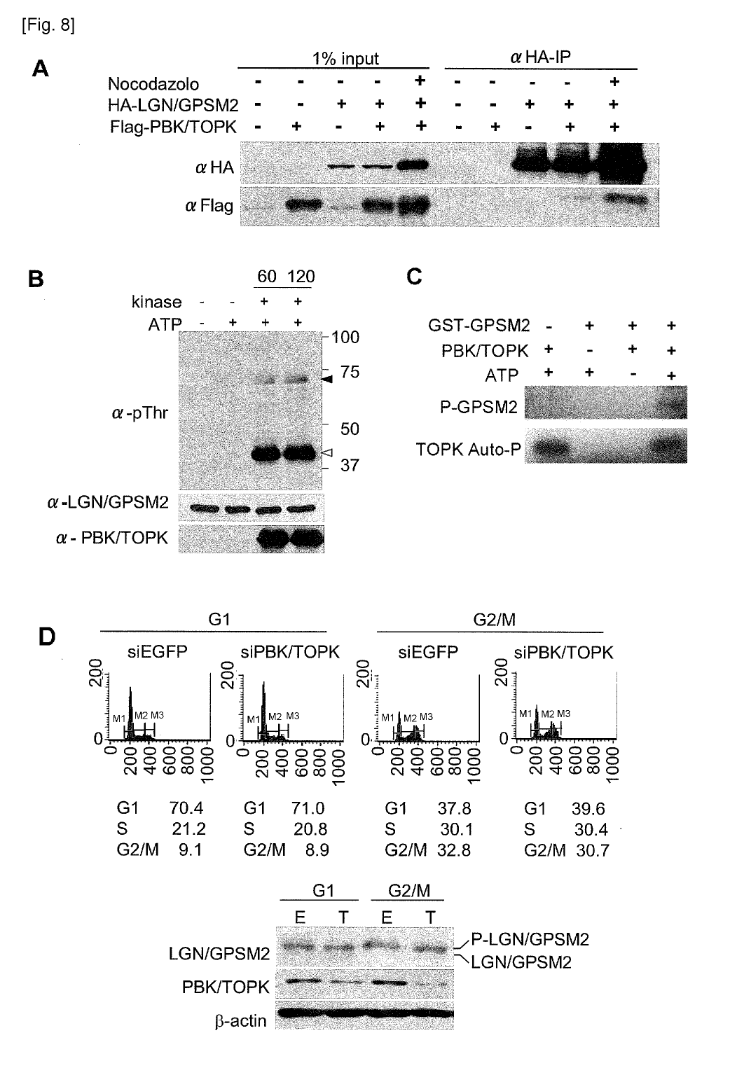

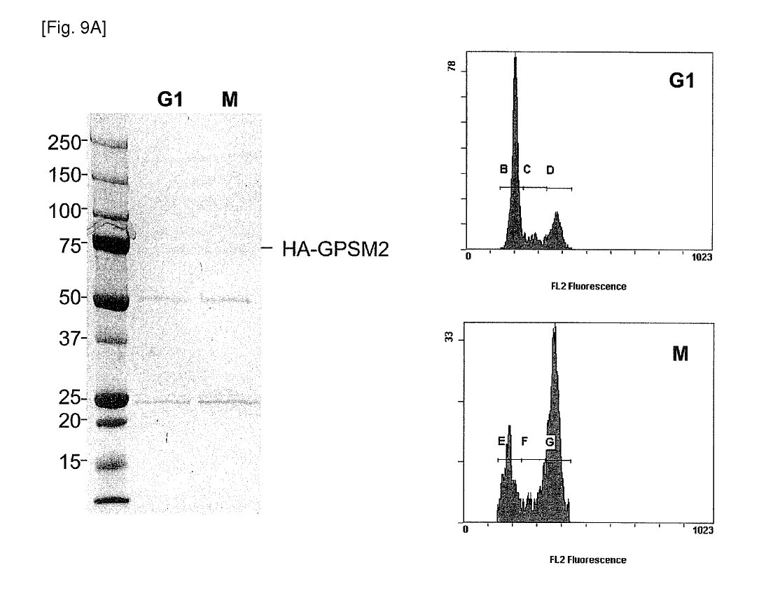

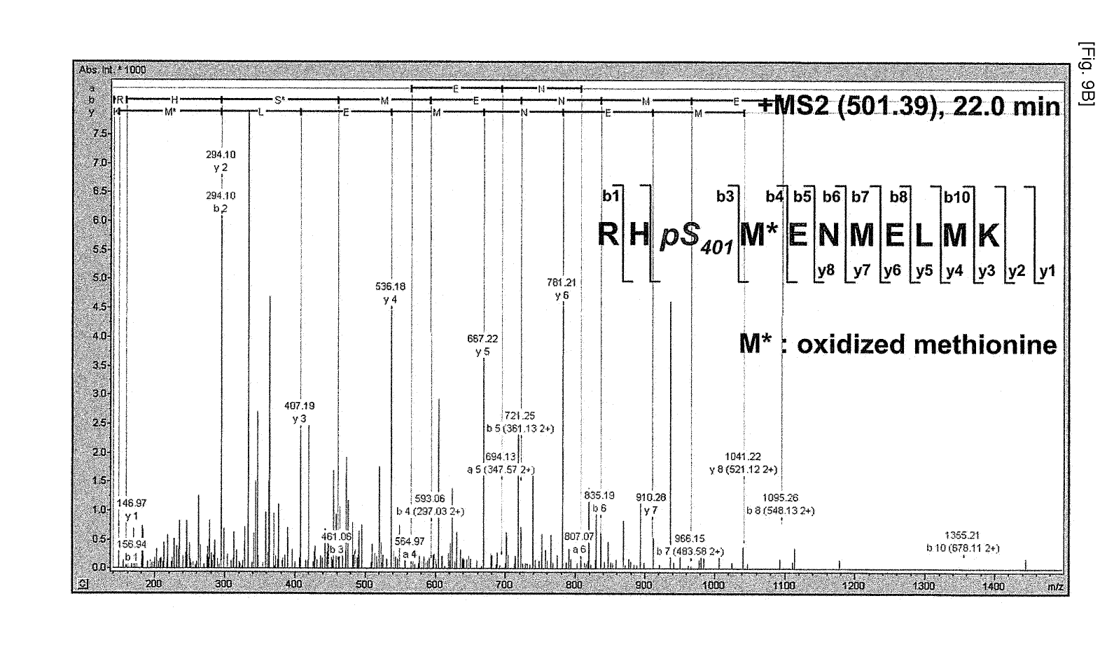

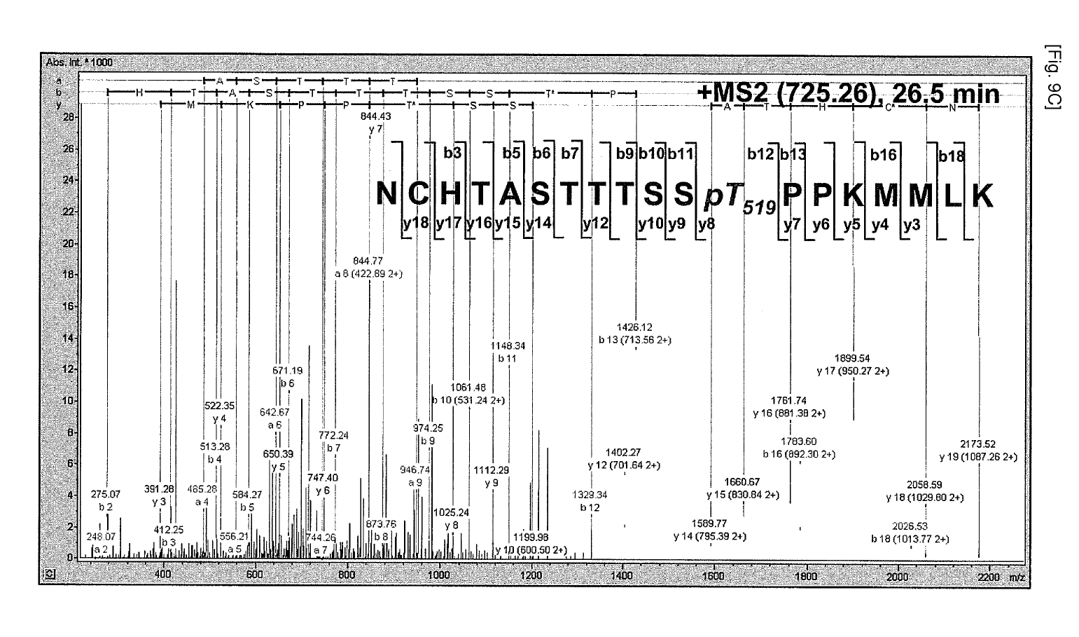

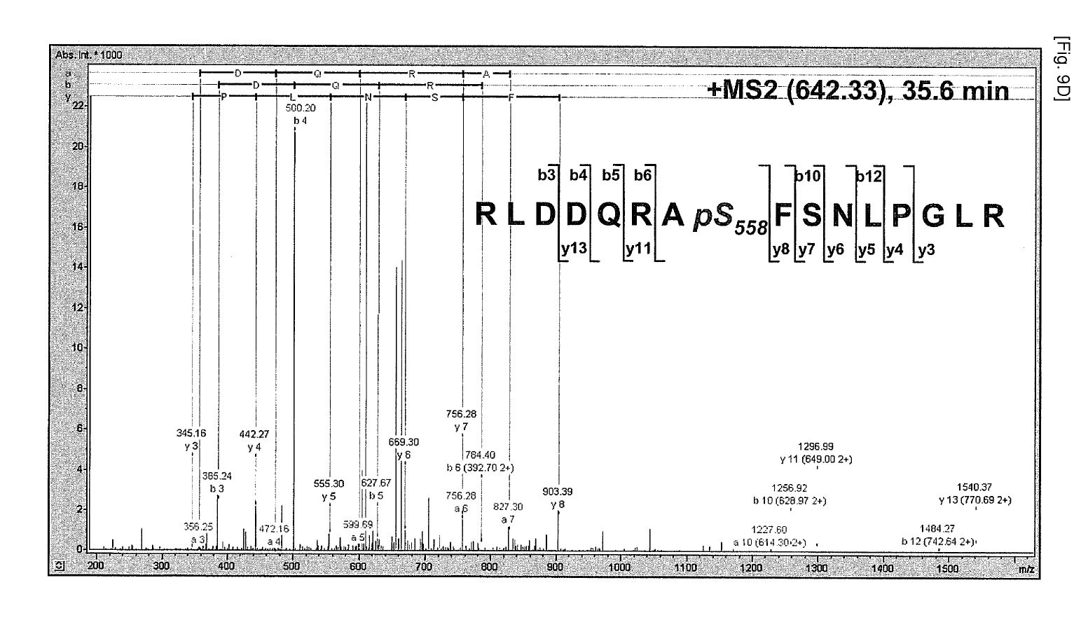

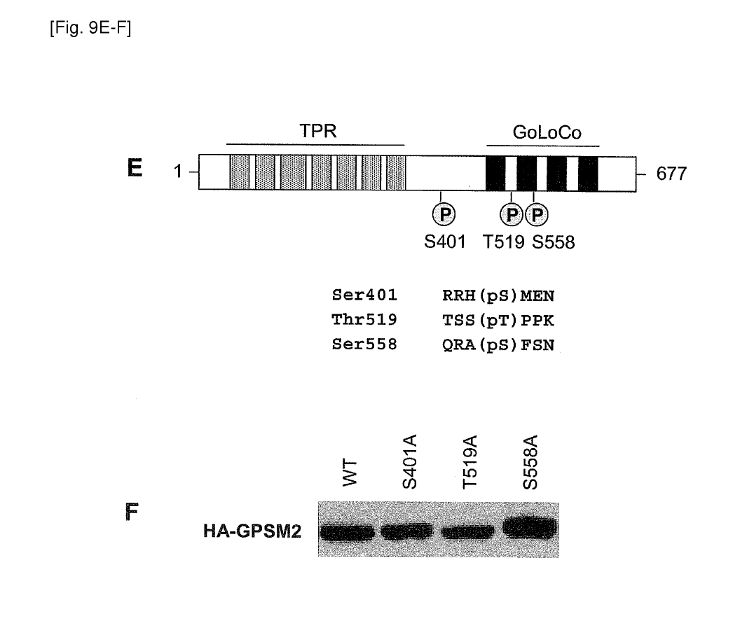

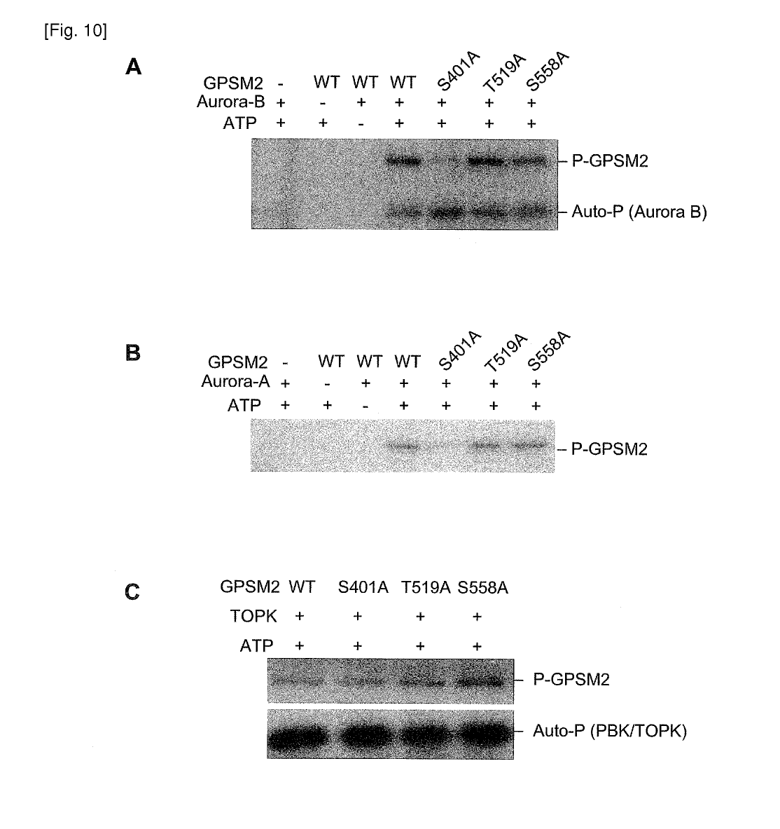

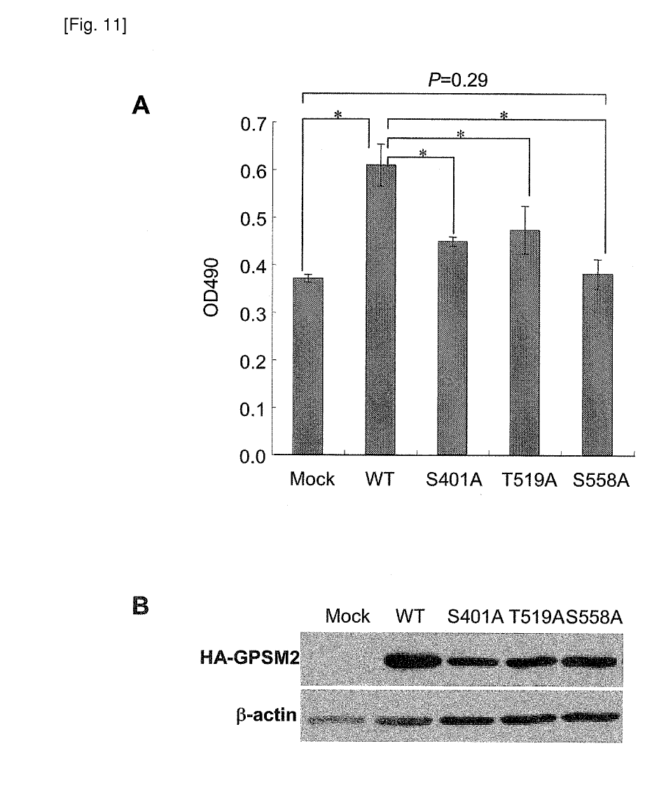

### BRIEF DESCRIPTION OF DRAWINGS

[FIG. 1] Expression pattern of LGN/GPSM2 in breast cancers and normal human organs. (A), Expression of LGN/GPSM2 in microdissected tumor cells from breast cancer tissues (42, 102, 247, 252, 255, 302, 473, 478, 502, 552, 646, 769, 779 and 780), compared with normal human tissues (MG; mammary gland) by semiquantitative RT-PCR. beta-actin served as a loading control. (B), Northern blot analysis of the two transcripts of LGN/GPSM2 in 20 breast cancer cell lines and normal human tissues including mammary gland, lung, heart, liver, kidney and brain. (C), Northern blot analysis of the LGN/GPSM2 transcript in various human tissues. (D), Genomic structure of two variants of LGN/GPSM2 (V1 and V2). Grey triangles indicate initiation codon, and black triangles indicate terminal codon. The number above each box indicates the exon number.

[FIG. 2] Immunocytochemical staining analysis. (A-C), T47D cells were stained using anti-LGN/GPSM2 antibody (red), co-stained with anti-alpha-tubulin (green;B) or F-actin affinity peptide, phalloidin (green;C). Nucleus were counterstained with 4′,6′-diamidine-2′-phenylindole dihydrochloride (DAPI; blue). Arrowheads indicate the midbody of the cytokinesis cells. Scale bar; 10 mcm.

[FIG. 3A-B] Cell-cycle dependent expression of LGN/GPSM2. (A), Fluorescence-activated cell sorting (FACS) analysis showed population of T47D cells collected at indicated time (0, 3, 6, 9, 12, 15, 18, 21, 24 h) after synchronization with aphidicolin treatment. (B), (Upper panels): Western blot analysis of endogenous LGN/GPSM2 at each cell cycle phase indicated in (A). Beta-actin is served as a quantitative control. (Lower panels): Semiquantitative RT-PCR analysis of LGN/GPSM2 at each cell cycle phase indicated in (A). Beta-actin is served as an internal quantitative control.

[FIG. 3C-E] (C), Fluorescence-activated cell sorting (FACS) analysis showed population of T47D cells collected at indicated time (0, 0.5, 1, 1.5, 2, 4, 6 h) after synchronization with nocodazole treatment. (D), Western blot analysis of endogenous LGN/GPSM2 at each cell cycle phase indicated in (C). Beta-actin is served as an internal quantitative control. (E), Lambda-protein phosphatase treatment of LGN/GPSM2. The cell lysates of nocodazole-treated T47D cells were incubated with lambda-protein phosphatase (+) or sodium fluoride (−) were analyzed by western blot. The phosphorylated and unphosphorylated LGN/GPSM2 proteins are indicated as P-LGN/GPSM2 and LGN/GPSM2, respectively.

[FIG. 4] Growth-inhibitory effects of LGN/GPSM2-siRNA in breast cancer cell lines, T47D (A-C, G-I) and BT20 (D-F). Semi-quantitative RT-PCR shows the expression of endogenous LGN/GPSM2 five days after transfection; expression of Beta2MG is served as an internal control (A, D, G). Colony-formation assays were performed 14 days after transfection (B, E, H) and MTT assays were performed 10 days after transfection (C, F, I). Shown data is a representative data of two independent analyses.

[FIG. 5] Effect of the LGN/GPSM2 overexpression on the cell growth examinded by bromodeoxyuridine incorporation assay (A, B) and by MTT assay (C,D). (A), Western blot analysis of HEK293 cells 48 h after transfection with empty vector (Mock) or pCAGGSnHA-LGN/GPSM2. Beta-actin is served as an internal quantitative control. (B), Bromodeoxyuridine (BrdUrd)-incorporation of HEK293 cells transfected with empty vector (Mock) or pCAGGSnHA-LGN/GPSM2 were measured. Shown data is a representative data of three independent analyses. (C), Western blot analysis of COS-7 cells 72 h after transfection with empty vector (Mock) or pCAGGSnHA-LGN/GPSM2. beta-actin is served as an internal quantitative control. (D), MTT assay of COS-7 cells transfected with empty vector (Mock) or pCAGGSnHA-LGN/GPSM2 were performed. Shown data is a representative data of three independent analyses.

[FIG. 6] Cell cycle analysis and morphological change of breast cancer cells transfected with LGN/GPSM2-siRNA oligonucleotide. (A), Western blot analysis of T47D cells transfected with siEGFP or siLGN/GPSM2. Samples were collected 24 h after transfection. NS: non-specific band. Beta-actin is served as an internal quantitative control. (B), FACS analysis showed the cell cycle population of T47D cells collected at 72 h after transfection. (C), Light microscopy images of T47D cells 72 h after transfection. Original magnification; ×100. Arrowheads indicate the aberrant intercellular bridges. Shown data is a representative data of two independent analyses.

[FIG. 7] Interaction of LGN/GPSM2 and TRIOBP/Tara. (A), Silver staining of SDS-PAGE gels that contained the immunoprecipitated products. (B), Immunoprecipitation analysis of HEK293 cells transfected with pCAGGSn3F-TRIOBP and pCAGGSnHA-LGN/GPSM2. Representative data of two independent experiments is shown. (C), Immunocytochemical staining of endogenous TRIOBP (red) and LGN/GPSM2 (green) in breast cancer cells, T47D. Cross-section image of midbody is shown in right two panels. Scale bar: 10 mcm. (D), Immunocytochemical staining of endogenous LGN/GPSM2 (red) and F-actin (green) in cytokinesis T47D cells transfected with siEGFP or siLGN/GPSM2 for 24 h. Scale bar: 10 mcm.

[FIG. 8] LGN/GPSM2 is phosphorylated by PBK/TOPK at G2/M phase. (A), Immunoprecipitation analysis of HEK293 cells transfected with pCAGGSn3F-PBK/TOPK and pCAGGSnHA-LGN/GPSM2. Representative data of two independent experiments is shown. (B), In vitro kinase assay was performed with purified full-length recombinant LGN/GPSM2 protein. Closed arrowhead indicates the phosphorylated LGN/GPSM2 and open arrowhead indicates the auto-phosphorylated PBK/TOPK. (C), In vitro kinase assay was performed with purified full-length recombinant GST-LGN/GPSM2 protein. Autoradiography images are shown. (D), (Upper panels): FACS analysis showed population of T47D cells transfected with siEGFP or siPBK/TOPK and collected at 0 h (G1) or 6 h (G2/M) after synchronization with aphidicolin treatment. (Lower panels): Western blot analysis of endogenous LGN/GPSM2 indicated above. Beta-actin is served as an internal quantitative control. The phosphorylated and unphosphorylated LGN/GPSM2 proteins are indicated as P-LGN/GPSM2 and LGN/GPSM2, respectively.

[FIG. 9A] LGN/GPSM2 is phosphorylated at Ser401, T519, and S558 in mitotic phase. (A) Immunoprecipitation of HA-LGN/GPSM2 from nocodazole-treated (M) MCF7 Tet-Off cells. Cell cycle analyses are shown below.

[FIG. 9B] (B) The assigned MS/MS spectra of LGN/GPSM2 399-409, 508-526, and 551-566 on Biotools Software are shown. Identified phosphorylated peptide sequences and matched b- or y-series ions are also displayed at the upper-right corner of each panel. pS or pT indicates the phosphorylated serine or threonine residues, respectively.

[FIG. 9C] (C) The assigned MS/MS spectra of LGN/GPSM2 399-409, 508-526, and 551-566 on Biotools Software are shown. Identified phosphorylated peptide sequences and matched b- or y-series ions are also displayed at the upper-right corner of each panel. pS or pT indicates the phosphorylated serine or threonine residues, respectively.

[FIG. 9D] (D) The assigned MS/MS spectra of LGN/GPSM2 399-409, 508-526, and 551-566 on Biotools Software are shown. Identified phosphorylated peptide sequences and matched b- or y-series ions are also displayed at the upper-right corner of each panel. pS or pT indicates the phosphorylated serine or threonine residues, respectively.

[FIG. 9E-F] (E) Schematic diagram of LGN/GPSM2 protein structure and phosphorylated amino acids identified here. (F) Western blotting of wild type (WT) and alanine-substituted (S401A, T519A, S558A) LGN/GPSM2. Samples were collected from transiently transfected HEK293 48hrs after transfection. Transfected cells were treated with 0.3 mcg/ml nocodazole for 18 hours in prior to collection.

[FIG. 10] Aurora kinase phosphorylates LGN/GPSM2 on serine residue 401 in vitro. GST-LGN/GPSM2 WT and alanine substitutes indicated were transferred to in vitro kinase reactions to test for phosphorylation by active Aurora kinase A (A), Aurora kinase B (B) and PBK/TOPK (C) in the presence of [gamma-³²P]-ATP. Phosphorylated proteins were visualized by autoradiography.

[FIG. 11] Serine 401, threonine 519 and serine 558 are involved in LGN/GPSM2-mediated growth enhancement. (A), MTT assay of COS-7 cells transfected with empty vector (Mock), pCAGGSnHA-LGN/GPSM2 WT or alanine substitutes indicated were performed. Shown data is a representative data of three independent analyses. (B), Western blot analysis of COS-7 cells 72 hours after transfection. beta-actin is served as an internal quantitative control.

### DESCRIPTION OF EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

Unless otherwise defined, all technical and scientific terms used herein have the same meaning as commonly understood by one of ordinary skill in the art to which this invention belongs. In case of conflict, the present specification, including definitions, will control.

Definitions

The words “a”, “an”, and “the” as used herein mean “at least one” unless otherwise specifically indicated.

The terms “isolated” and “purified” used in relation with a substance (e.g., polypeptide, antibody, polynucleotide, etc.) indicates that the substance is substantially free from at least one substance that may else be included in the natural source. Thus, an isolated or purified antibody refers to antibodies that is substantially free of cellular material such as carbohydrate, lipid, or other contaminating proteins from the cell or tissue source from which the protein (antibody) is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, it is also preferably substantially free of culture medium, which includes preparations of polypeptide with culture medium less than about 20%, 10%, or 5% of the volume of the protein preparation. When the polypeptide is produced by chemical synthesis, it is preferably substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In a preferred embodiment, antibodies and polypeptides of the present invention are isolated or purified. An “isolated” or “purified” nucleic acid molecule, such as a cDNA molecule, can be substantially free of other cellular material, or culture medium when produced by recombinant techniques, or substantially free of chemical precursors or other chemicals when chemically synthesized. In a preferred embodiment, nucleic acid molecules encoding antibodies of the present invention are isolated or purified.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers. The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “polynucleotides”, “oligonucleotide”, “nucleotides”, “nucleic acids”, and “nucleic acid molecules” are used interchangeably unless otherwise specifically indicated and are similarly to the amino acids referred to by their commonly accepted single-letter codes. Similar to the amino acids, they encompass both naturally-occuring and non-naturally occurring nucleic acid polymers. The polynucleotide, oligonucleotide, nucleotides, nucleic acids, or nucleic acid molecules may be composed of DNA, RNA or a combination thereof.

As use herein, the term “double-stranded molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As used herein, the term “dsRNA” refers to a construct of two RNA molecules comprising complementary sequences to one another and that have annealed together via the complementary sequences to form a double-stranded RNA molecule. The nucleotide sequence of two strands may comprise not only the “sense” or “antisense” RNAs selected from a protein coding sequence of target gene sequence, but also RNA molecule having a nucleotide sequence selected from non-coding rigion of the target gene.

The term “shRNA”, as used herein, refers to an siRNA having a stem-loop structure, comprising a first and second regions complementary to one another, i.e., sense and antisense strands. The degree of complementarity and orientation of the regions is sufficient such that base pairing occurs between the regions, the first and second regions is joined by a loop region, and the loop results from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotied composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a sense nucleic acid sequence (also referred to as “sense strand”), an antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules comprising complementary sequences to one another and that have annealed together via the complementary sequences to form a double-stranded polynucleotide molecule. The nucleotide sequence of two strands may comprise not only the “sense” or “antisense” polynucleotides sequence selected from a protein coding sequence of target gene sequence, but also polynucleotide having a nucleotide sequnence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, comprising a first and second regions complementary to one another, i.e., sense and antisense strands. The degree of complementarity and orientation of the regions is sufficient such that base pairing occurs between the regions, the first and second regions is joined by a loop region, the loop results from a lack of base pairing between nucleotides (or nucleotide analogs) within the loop region. The loop region of an shD/R-NA is a single-stranded region intervening between the sense and antisense strands and may also be referred to as “intervening single-strand”.

I. Polynucleotides and Polypeptides

The present invention is based in part on the discovery of elevated expression of the LGN/GPSM2 gene in cancer cells. The expression of the gene was discovered to be particularly elevated in clinical cancer tissues.

Nucleotide sequences of the genes and amino acid sequences of the polypeptides recited to the present invention, are known to those skilled in the art, and obtained, for example, from gene databases on the web site such as GenBank™.

For example, exemplary nucleotide sequences of human LGN/GPSM2 gene are shown in SEQ ID NO: 39(variant1), SEQ ID NO: 41(variant2), and SEQ ID NO:52 and these sequences are also available as GenBank Accession No. AB445462, NM_{—}013296, and U54999 respectively. Both of variants share same ORF (from 1^{st} to 2586^{th} position in the both of variants) encoding same amino acid sequence(SEQ ID NO: 40). Herein, the nucleotide sequence shown in SEQ ID NO: 39 is a novel sequence. Exemplary nucleotide sequence of human TRIOBP/Tara (TRIO and F-actin binding protein) gene is shown in SEQ ID NO: 42 and this sequence is also available as GenBank Accession No. NM_{—}001039141. Exemplary nucleotide sequences of human PBK/TOPK (PDZ binding kinase) gene is shown in SEQ ID NO: 44 and this sequence is also available as GenBank Accession No. AF237709. Herein, the LGN/GPSM2, TRIOBP/Tara or PBK/TOPK genes encompasses the human genes as well as gene homologs of other animals including non-human primate, mouse, rat, dog, cat, horse, and cow but are not limited thereto, and includes allelic mutants and genes found in other animals as corresponding to the individual gene. In some embodiments, the LGN/GPSM2 gene shares at least about 90%, 93%, 95%, 97%, 99% sequence identity with the human LGN/GPSM2 gene of SEQ ID NOs:39, 41, or 52, as measured using a sequence comparison algorithm known in art, e.g., BLAST or ALIGN, set to default settings. Similarly, the TRIOBP/Tara, PBK/TOPK shares at least about 90%, 93%, 95%, 97%, 99% sequence identity with the nucleotide sequences of SEQ ID NOs: 42 and 44, respectively.

Exemplary amino acid sequence encoded the human LGN/GPSM2 gene is shown in SEQ ID NO: 40, or 53 (Genbank Accession No. AAB40385). Exemplary amino acid sequence encoded the human TRIOBP/Tar gene is shown in SEQ ID NO: 43. Exemplary amino acid sequence encoded the human PBK/TOPK, gene is shown in SEQ ID NO: 45.

In the present invention, the polypeptide encoded by the LGN/GPSM2 gene is referred to as “LGN/GPSM2”, and sometimes as “LGN/GPSM2 polypeptide” or “LGN/GPSM2 protein”. The other polypeptides are also referred to in the same manner.

According to an aspect of the present invention, functional equivalents are also included in the LGN/GPSM2, TRIOBP/Tara or PBK/TOPK polypeptides. Herein, a “functional equivalent” of a protein is a polypeptide that has a biological activity equivalent to the protein. Namely, any polypeptide that retains the biological ability of the original protein may be used as such a functional equivalent in the present invention.

Such functional equivalents include those wherein one or more amino acids are substituted, deleted, added, or inserted to the natural occurring amino acid sequence of the original protein. Alternatively, the polypeptide may be one that comprises an amino acid sequence having at least about 80%, 90%, 93%, 95%, 97% or 99% homology (also referred to as sequence identity) to the sequence of the respective proteins. In other embodiments, the polypeptide can be encoded by a polynucleotide that hybridizes under stringent conditions to the naturally occurring nucleotide sequence of the LGN/GPSM2, TRIOBP/Tara or PBK/TOPK gene.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10 degrees C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions can be as following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

Generally, it is known that modifications of one or more amino acid in a protein do not influence the function of the protein. One of skill in the art will recognize that individual additions, deletions, insertions, or substitutions to an amino acid sequence which alters a single amino acid or a small percentage of amino acids is a “conservative modification” wherein the alteration of a protein results in a protein with similar functions. Conservative substitution tables providing functionally similar amino acids are well known in the art. For example, the following eight groups each contain amino acids that are conservative substitutions for one another:

1) Alanine (A), Glycine (G);

2) Aspartic acid (d), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cystein (C), Methionine (M) (see, e.g., Creighton, Proteins 1984).

Such conservatively modified polypeptides are included in the polypeptides recited in the present invention (i.e., LGN/GPSM2, TRIOBP/Tara and PBK/TOPK polypeptides). However, the present invention is not restricted thereto and the polypeptides includes non-conservative modifications so long as they retain at least one biological activity of the original protein. Furthermore, the modified proteins do not exclude polymorphic variants, interspecies homologues, and those encoded by alleles of these proteins.

Moreover, the LGN/GPSM2 gene of the present invention encompasses polynucleotides that encode such functional equivalents of the LGN/GPSM2 protein. Similarly, TRIOBP/Tara and PBK/TOPK gene encompasses polynucleotides that encode such functional equivalents of the TRIOBP/Tara and PBK/TOPK proteins, respectively.

II. Diagnosing Cancer:

II-1. Method for Diagnosing Cancer or a Predisposition for Developing Cancer

The expression of the LGN/GPSM2 gene was found to be specifically elevated in patients with cancer. Therefore, the gene identified herein as well as its transcription and translation products find diagnostic utility as a marker for cancer and by measuring the expression of the LGN/GPSM2 gene in a cell sample, cancer can be diagnosed.

Specifically, the present invention provides a method for diagnosing cancer or a predisposition for developing cancer in a subject by determining the expression level of the LGN/GPSM2 gene in the subject. In some embodiments, the expression level of the LGN/GPSM2 gene is determined in breast tissue from the subject.

Alternatively, the present invention provides a method for detecting cancer cells in a subject-derived breast tissue sample, said method comprising the step of determining the expression level of the LGN/GPSM2 gene in a subject-derived breast tissue sample, wherein an increase in said expression level as compared to a normal control level of said gene indicates the presence or suspicion of cancer cell in the tissue.

Such result may be combined with additional information to assist a doctor, nurse, or other practitioner to diagnose that a subject suffers from the disease. Alternatively, the present invention may provide a doctor with useful information to diagnose that the subject suffers from the disease. For example, according to the present invention, when the suspicion or doubt of the presence of cancer cells in the tissue obtained from a subject is indicated, clinical decisions would be made by a doctor with consideration of this observation and another aspect including the pathological finding of the tissue, levels of known tumor marker(s) in blood, or clinical course of the subject, etc. Some blood tumor markers use for the diagnostis of breast cancer are well known. For example, carbohydrate antigen 125 (CA125), carbohydrate antigen 15-3 (CA15-3), or carcinoembryonic antigen (CEA) are known blood tumor markers for breast cancer. According to the present invention, an intermediate result for examining the condition of a subject may also be provided by measuring the levels of LGN/GPSM2 protein in a patient.

In another embodiment, the present invention provides a method for detecting a diagnostic marker of cancer, said method comprising the step of detecting the expression of the LGN/GPSM2 gene in a subject-derived biological sample as a diagnostic marker of cancer. Preferable cancers to be diagnosed by the present method include breast cancer.

In the context of the present invention, the term “diagnosing” is intended to encompass predictions and likelihood analysis. The present method is intended to be used clinically in making decisions concerning treatment modalities, including therapeutic intervention, diagnostic criteria such as disease stages, and disease monitoring and surveillance for cancer. According to the present invention, an intermediate result for examining the condition of a subject may also be provided. Such intermediate result may be combined with additional information to assist a doctor, nurse, or other practitioner to diagnose that a subject suffers from the disease. Alternatively, the present invention may be used to detect cancerous cells in a subject-derived tissue, and provide a doctor with useful information to diagnose that the subject suffers from the disease.

A subject to be diagnosed by the present method is preferably a mammal. Exemplary mammals include, but are not limited to, human, non-human primate, mouse, rat, dog, cat, horse, and cow.

It is preferred to collect a biological sample from the subject to be diagnosed to perform the diagnosis. Any biological material can be used as the biological sample for the determination so long as it comprises the objective transcription or translation product of the LGN/GPSM2 gene. The biological samples include, but are not limited to, bodily tissues and fluids, such as blood, plasma, serum, saliva, sputum, and urine. Preferably, the biological sample contains a cell population comprising an epithelial cell, more preferably a cancerous breast epithelial cell or a breast epithelial cell derived from tissue suspected to be cancerous. Further, if necessary or desired, the cell may be purified from the obtained bodily tissues and fluids, and then used as the biological sample.

According to the present invention, the expression level of the LGN/GPSM2 gene is determined in the subject-derived biological sample. The expression level can be determined at the transcription (nucleic acid) product level, using any method known in the art. For example, the mRNA of the LGN/GPSM2 gene may be quantified using probes by hybridization methods (e.g., Northern hybridization) or using quantitative nucleic acid amplification techniques. The detection also may be carried out on a chip or an array. The use of an array is preferable for detecting the expression level of a plurarity of genes (e.g., various cancer specific genes) including the present LGN/GPSM2 gene. Those skilled in the art can prepare such probes utilizing the sequence information of the LGN/GPSM2 gene (e.g., SEQ ID NO: 39; GenBank Accession No. AB445462 or SEQ ID NO: 41; GenBank Accession No. NM_{—}013296). For example, the cDNA of the LGN/GPSM2 gene or the fragment may be used as the probes, such as Hs.659320 (SEQ ID NO: 38; GenBank Accession No. AK000053.1). If necessary or desired, the probe may be labeled with a suitable label, such as dyes and isotopes, and the expression level of the gene may be detected as the intensity of the hybridized labels.

Furthermore, the transcription product of the LGN/GPSM2 gene may be quantified using primers by amplification-based detection methods (e.g., RT-PCR). Such primers can also be prepared based on the available sequence information of the gene. For example, the primers used in the Example (SEQ ID NOs:3 and 4) may be employed for the detection by RT-PCR, but the present invention is not restricted thereto.

Specifically, a probe or primer used for the present method hybridizes under stringent, moderately stringent, or low stringent conditions to the mRNA of the LGN/GPSM2 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degrees C. lower than the thermal melting point (Tₘ) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees C. for short probes or primers (e.g., 10 to 50 nucleotides) and at least about 60 degrees C. for longer probes or primers. Stringent conditions may also be achieved with the addition of destabilizing agents, such as formamide.

Alternatively, the translation product may be detected for the diagnosis of the present invention. For example, the quantity of the LGN/GPSM2 protein may be determined. A method for determining the quantity of the protein as the translation product includes immunoassay methods that use an antibody specifically recognizing the protein. The antibody may be monoclonal or polyclonal. Furthermore, any fragment or modification (e.g., chimeric antibody, scFv, Fab, F(ab′)₂, Fv, etc.) of the antibody may be used for the detection, so long as the fragment retains the binding ability to the LGN/GPSM2 protein. Methods to prepare these kinds of antibodies for the detection of proteins are well known in the art (e.g., see EXAMPLE I), and any method may be employed in the present invention to prepare such antibodies and equivalents thereof.

As another method to detect the expression level of the LGN/GPSM2 gene based on its translation product, the intensity of staining may be observed via immunohistochemical analysis using an antibody against the LGN/GPSM2 protein. Namely, the observation of strong staining indicates increased presence of the protein and at the same time high expression level of the LGN/GPSM2 gene.

Furthermore, the translation product may be detected based on its biological activity. The cancer cell growth promoting ability of the LGN/GPSM2 protein may be used as an index of the LGN/GPSM2 protein existing in the biological sample.

Moreover, in addition to the expression level of the LGN/GPSM2 gene, the expression level of other cancer-associated genes, for example, genes known to be differentially expressed in breast cancer, may also be determined to improve the accuracy of the diagnosis.

The expression level of the LGN/GPSM2 gene in a biological sample can be considered to be increased if it increases from the normal control level of the LGN/GPSM2 gene by, for example, 10%, 25%, or 50%; or increases to more than 1.1 fold, more than 1.5 fold, more than 2.0 fold, more than 5.0 fold, more than 10.0 fold, or more.

The control level may be determined at the same time with the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of the LGN/GPSM2 gene in samples from subjects whose disease states are known. Furthermore, the control level can be a database of expression patterns from previously tested cells. Moreover, according to an aspect of the present invention, the expression level of the LGN/GPSM2 gene in a biological sample may be compared to multiple control levels, which control levels are determined from multiple reference samples. It is preferred to use a control level determined from a reference sample derived from a tissue type similar to that of the patient-derived biological sample, e.g., epithelial breast tissue. Moreover, it is preferred, to use the standard value of the expression levels of the LGN/GPSM2 gene in a population with a known disease state. The standard value may be obtained by any method known in the art. For example, a range of mean plus/minus 2 S.D. or mean plus/minus 3 S.D. may be used as standard value.

In the context of the present invention, a control level determined from a biological sample that is known not to be cancerous is called “normal control level”. On the other hand, if the control level is determined from a cancerous biological sample, it will be called “cancerous control level”.

When the expression level of the LGN/GPSM2 gene is increased compared to the normal control level or is similar to the cancerous control level, the subject may be diagnosed to be suffering from or at a risk of developing cancer. Furthermore, in cases where the expression levels of multiple cancer-related genes are compared, a similarity in the gene expression pattern between the sample and the reference which is cancerous indicates that the subject is suffering from or at a risk of developing cancer.

Differences between the expression levels of a test biological sample and the control level can be normalized to the expression level of control nucleic acids, e.g. housekeeping genes. The expression levels of housekeeping genes are known not to differ depending on the cancerous or non-cancerous state of the cell. Exemplary control genes include, but are not limited to, beta actin, glyceraldehyde 3 phosphate dehydrogenase, and ribosomal protein P1.

II-2. Assessing Efficacy of Cancer Treatment

The differential expression of the LGN/GPSM2 gene between normal and cancerous cells also allows for the course of treatment of cancers to be monitored, and the above-described method for diagnosing cancer can be applied for assessing the efficacy of a treatment on cancer. Specifically, the efficacy of a treatment on cancer can be assessed by determining the expression level of the LGN/GPSM2 gene in a cell(s) derived from a subject undergoing the treatment. If desired, test cell populations are obtained from the subject at various time points, before, during, and/or after the treatment. The expression level of the LGN/GPSM2 gene can be, for example, determined following the method described above under the item of ‘I-1. Method for diagnosing cancer or a pre-disposition for developing cancer’. In the context of the present invention, it is preferred that the control level to which the detected expression level is compared is determined from the LGN/GPSM2 gene expression in a cell(s) not exposed to the treatment of interest.

If the expression level of the LGN/GPSM2 gene is compared to a control level that is determined from a normal cell or a cell population containing no cancer cells, a similarity in the expression level indicates that the treatment of interest is efficacious and an increase in the expression level indicates a less favorable clinical outcome or prognosis of that treatment. On the other hand, if the comparison is conducted against a control level that is determined from a cancer cell or a cell population containing a cancer cell(s), a decrease in the expression level indicates efficacious treatment, while a similarity in the expression level indicates a less favorable clinical outcome or prognosis.

Furthermore, the expression levels of the LGN/GPSM2 gene before and after a treatment can be compared according to the present method to assess the efficacy of the treatment. Specifically, the expression level detected in a subject-derived biological sample after a treatment (i.e., post-treatment level) is compared to the expression level detected in a biological sample obtained prior to treatment onset from the same subject (i.e., pre-treatment level). A decrease in the post-treatment level compared to the pre-treatment level indicates that the treatment of interest is efficacious while an increase in or similarity of the post-treatment level to the pre-treatment level indicates a less favorable clinical outcome or prognosis.

As used herein, the term “efficacious” indicates that the treatment leads to a reduction in the expression of a pathologically up-regulated gene, an increase in the expression of a pathologically down-regulated gene or a decrease in size, prevalence, or metastatic potential of carcinoma in a subject. When a treatment of interest is applied prophylactically, “efficacious” means that the treatment retards or prevents the forming of tumor or retards, prevents, or alleviates at least one clinical symptom of cancer. Assessment of the state of tumor in a subject can be made using standard clinical protocols.

In addition, efficaciousness of a treatment can be determined in association with any known method for diagnosing cancer. Cancers can be diagnosed, for example, by identifying symptomatic anomalies, e.g., weight loss, abdominal pain, back pain, anorexia, nausea, vomiting and generalized malaise, weakness, and jaundice. Cancers also can be diagnosed by pathological evaluation of tissue architecture.

II-3. Assessing Prognosis of Subject with Cancer

The methods for diagnosing cancer described above can also be used for assessing the prognosis of cancer in a subject. Thus, the present invention also provides methods for assessing the prognosis of a subject with breast cancer. The expression level of the LGN/GPSM2 gene can be, for example, determined following the method described above under the item of ‘II-1. Method for diagnosing cancer or a predisposition for developing cancer’. For example, the expression level of the LGN/GPSM2 gene in biological samples derived from patients over a spectrum of disease stages can be used as control levels to be compared with the expression level of the gene detected for a subject. By comparing the expression level of the LGN/GPSM2 gene in a subject and the control level(s) the prognosis of the subject can be assessed. Alternatively, by comparing over time the pattern of expression levels in a subject, the prognosis of the subject can be assessed.

For example, an increase in the expression level of LGN/GPSM2 gene in a subject as compared to a normal control level indicates less favorable prognosis. Conversely, a similarity in the expression level as compared to normal control level indicates a more favorable prognosis for the subject.

III. Kits:

The present invention also provides reagents for detecting cancer, i.e., reagents useful for detecting the transcription or translation product of the LGN/GPSM2 gene. Examples of such reagents include those capable of:

(a) detecting mRNA of the LGN/GPSM2 gene;

(b) detecting the LGN/GPSM2 protein; and/or

(c) detecting the biological activity of the LGN/GPSM2 protein in a subject-derived biological sample.

Suitable reagents include nucleic acids that specifically bind to or identify a transcription product of the LGN/GPSM2 gene. For example, the nucleic acids that specifically bind to or identify a transcription product of the LGN/GPSM2 gene include without limitation oligonucleotides (e.g., probes and primers) having a sequence that is complementary to a portion of the LGN/GPSM2 gene transcription product. Such oligonucleotides are exemplified by primers and probes that are specific to the mRNA of the gene of interest and may be prepared based on methods well known in the art. Alternatively, antibodies are exemplary reagents for detecting the translation product of the gene. The probes, primers, and antibodies described above under the item of ‘I-1. Method for diagnosing cancer or a predisposition for developing cancer’ are suitable examples of such reagents.

The LGN/GPSM2 translation products may also be detected based on biological activity. The present invention identifies that LGN/GPSM2 interacts with TRIOBP/tera or PBK/TOPK in breast cancer cells. Furthermore, the phosphorylation of LGN/GPSM2 by PBK/TOPK is also described. Any method known in the art can be used for detecting the biological activity of LGN/GPSM2 translation products.

The present kit find use for detecting breast cancer.

These reagents may be used for the above-described diagnosis of cancer. Exemplary assay formats for using the reagents include Northern hybridization or sandwich ELISA, both of which are well-known in the art.

The detection reagents may be packaged together in the form of a kit. For example, the detection reagents may be packaged in separate containers. Furthermore, the detection reagents may be packaged with other reagents necessary for the detection. For example, a kit may include a nucleic acid or antibody (e.g., either bound to a solid matrix or packaged separately with reagents for binding them to the matrix) as the detection reagent, a control reagent (positive and/or negative), and/or a detectable label. A kit of the present invention may further include other materials desirable from a commercial and user standpoint, including buffers, diluents, filters, needles, syringes. These reagents and such may be retained in a container with a label. Suitable containers include bottles, vials, and test tubes. The containers may be formed from a variety of materials, such as glass or plastic. Instructions (e.g., written, auditory or visual, e.g., print-out, tape recording, VCR, DVD, CD-ROM, etc.) for carrying out the assay may also be included in the kit.

Although the present kit is suited for the detection and diagnosis of breast cancer, it may also be useful in assessing the prognosis of cancer and/or monitoring the efficacy of a cancer therapy.

As an aspect of the present invention, the reagents for detecting cancer may be immobilized on a solid matrix, for example, a porous strip or an array, to form at least one site for detecting cancer. The measurement or detection region of a porous strip may include a plurality of sites, each containing a detection reagent (e.g., nucleic acid). A test strip may also contain sites for negative and/or positive controls. Alternatively, control sites may be located on a separate strip from the test strip. Optionally, the different detection sites may contain different amounts of immobilized detection reagents (e.g., nucleic acid), i.e., a higher amount in the first detection site and lesser amounts in subsequent sites. Upon the addition of test biological sample, the number of sites displaying a detectable signal provides a quantitative indication of the expression level of the LGN/GPSM2 gene in the sample. The detection sites may be configured in any suitably detectable shape and are typically in the shape of a bar or dot spanning the width of a test strip.

IV. Screening Methods:

Using the LGN/GPSM2 gene, polypeptides encoded by the gene or fragments thereof, or transcriptional regulatory region of the gene, one can screen agents that alter the expression of the gene or the biological activity of a polypeptide encoded by the gene. Such agents can be used as pharmaceuticals for treating or preventing cancer, in particular, breast cancer. Thus, the present invention provides methods of screening for agents for treating or preventing cancer using the LGN/GPSM2 gene, polypeptide encoded by the gene or fragments thereof, or transcriptional regulatory region of the gene.

An agent isolated by the screening method of the present invention is an agent that is expected to inhibit the expression of the LGN/GPSM2 gene or the activity of the translation product of the gene, and thus, is a candidate for treating or preventing diseases attributed to overexpression of LGN/GPSM2. The agents are expected to be particularly suited for the treatment or prevention of cancers that relates to the overexpression of LGN/GPSM2. Namely, the agents screened through the present methods are deemed to have a clinical benefit and can be further tested for its ability to prevent cancer cell growth in animal models or test subjects. Although the agents or compounds obtained by present screening methods may be applied to any cancers in which LGN/GPSM2 is overexpressed, suitable cancer is breast cancer.

In the context of the present invention, agents to be identified through the present screening methods may be any compound or composition including several compounds. Furthermore, the test agent exposed to a cell or protein according to the screening methods of the present invention may be a single compound or a combination of compounds. When a combination of compounds is used in the methods, the compounds may be contacted sequentially or simultaneously.

Any test agent, for example, cell extracts, cell culture supernatants, products of fermenting microorganism, extracts from marine organism, plant extracts, purified or crude proteins, peptides, non-peptide compounds, synthetic micromolecular compounds (including nucleic acid constructs, such as antisense RNA, siRNA, Ribozymes, etc.) and natural compounds can be used in the screening methods of the present invention. The test agent of the present invention also can be obtained using any of the numerous approaches in combinatorial library methods known in the art, including

(1) biological libraries,

(2) spatially addressable parallel solid phase or solution phase libraries,

(3) synthetic library methods requiring deconvolution,

(4) the “one-bead one-compound” library method and

(5) synthetic library methods using affinity chromatography selection.

The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698, 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the compound screened by any of the present screening methods is converted by addition, deletion and/or replacement, is included in the agents obtained by the screening methods of the present invention.

Furthermore, when the screened test agent is a protein, for obtaining a DNA encoding the protein, either the whole amino acid sequence of the protein may be determined to deduce the nucleic acid sequence coding for the protein, or partial amino acid sequence of the obtained protein may be analyzed to prepare an oligo DNA as a probe based on the sequence, and screen cDNA libraries with the probe to obtain a DNA encoding the protein. The obtained DNA finds use in preparing the test agent which is a candidate for treating or preventing cancer.

IV-1. Protein Based Screening Methods

According to the present invention, the expression of the LGN/GPSM2 gene has been found to be associated with the growth and/or survival of cancer cells, in particular breast cancer cells. Therefore, it was considered that agents which suppress the function of the LGN/GPSM2 polypeptide encoded by the LGN/GPSM2 gene inhibit the growth and/or survival of breast cancer cells, and find use in treating or preventing breast cancer. Thus, the present invention provides methods of screening an agent for treating or preventing breast cancer, using the LGN/GPSM2 polypeptide.

In addition to the LGN/GPSM2 polypeptide, fragments of the polypeptide may be used for the present screening so long as it retains at least one biological activity of the naturally occurring LGN/GPSM2 polypeptide.

The LGN/GPSM2 polypeptide or fragments thereof may be further linked to other substances so long as the polypeptide and fragments retains at least one of its biological activity. Usable substances include: peptides, lipids, sugar and sugar chains, acetyl groups, natural and synthetic polymers, etc. These kinds of modifications may be performed to confer additional functions or to stabilize the polypeptide and fragments.

The LGN/GPSM2 polypeptide or fragments used for the present method may be obtained from nature as naturally occurring proteins via conventional purification methods or through chemical synthesis based on the selected amino acid sequence. For example, conventional peptide synthesis methods that can be adopted for the synthesis include:

1) Peptide Synthesis, Interscience, New York, 1966;

2) The Proteins, Vol. 2, Academic Press, New York, 1976;

3) Peptide Synthesis (in Japanese), Maruzen Co., 1975;

4) Basics and Experiment of Peptide Synthesis (in Japanese), Maruzen Co., 1985;

5) Development of Pharmaceuticals (second volume) (in Japanese), Vol. 14 (peptide synthesis), Hirokawa, 1991;

6) WO99/67288; and

7) Barany G. & Merrifield R. B., Peptides Vol. 2, “Solid Phase Peptide Synthesis”, Academic Press, New York, 1980, 100-118.

Alternatively, the LGN/GPSM2 protein may be obtained adopting any known genetic engineering methods for producing polypeptides (e.g., Morrison J., J Bacteriology 1977, 132: 349-51; Clark-Curtiss & Curtiss, Methods in Enzymology (eds. Wu et al.) 1983, 101: 347-62). For example, first, a suitable vector comprising a polynucleotide encoding the objective protein in an expressible form (e.g., downstream of a regulatory sequence comprising a promoter) is prepared, transformed into a suitable host cell, and then the host cell is cultured to produce the protein. More specifically, a gene encoding the LGN/GPSM2 polypeptide is expressed in host (e.g., animal) cells and such by inserting the gene into a vector for expressing foreign genes, such as pSV2neo, pcDNA I, pcDNA3.1, pCAGGS, or pCD8. A promoter may be used for the expression. Any commonly used promoters may be employed including, for example, the SV40 early promoter (Rigby in Williamson (ed.), Genetic Engineering, vol. 3. Academic Press, London, 1982, 83-141), the EF-alpha promoter (Kim et al., Gene 1990, 91:217-23), the CAG promoter (Niwa et al., Gene 1991, 108:193), the RSV LTR promoter (Cullen, Methods in Enzymology 1987, 152:684-704), the SR alpha promoter (Takebe et al., Mol Cell Biol 1988, 8:466), the CMV immediate early promoter (Seed et al., Proc Natl Acad Sci USA 1987, 84:3365-9), the SV40 late promoter (Gheysen et al., J Mol Appl Genet 1982, 1:385-94), the Adenovirus late promoter (Kaufman et al., Mol Cell Biol 1989, 9:946), the HSV TK promoter, and such. The introduction of the vector into host cells to express the LGN/GPSM2 gene can be performed according to any methods, for example, the electroporation method (Chu et al., Nucleic Acids Res 1987, 15:1311-26), the calcium phosphate method (Chen et al., Mol Cell Biol 1987, 7:2745-52), the DEAE dextran method (Lopata et al., Nucleic Acids Res 1984, 12:5707-17; Sussman et al., Mol Cell Biol 1985, 4:1641-3), the Lipofectin method (Derijard B, Cell 1994, 7:1025-37; Lamb et al., Nature Genetics 1993, 5:22-30; Rabindran et al., Science 1993, 259:230-4), and such.

The LGN/GPSM2 protein may also be produced in vitro adopting an in vitro translation system.

The LGN/GPSM2 polypeptide to be contacted with a test agent can be, for example, a purified polypeptide, a soluble protein, or a fusion protein fused with other polypeptides.

IV-1-1. Identifying Agents that Bind to LGN/GPSM2 Polypeptide

An agent that binds to a LGN/GPSM2 protein is likely to alter the expression of the gene coding for the protein or the biological activity of the protein. Thus, as an aspect, the present invention provides a method of screening an agent for treating or preventing breast cancer, which comprises the steps of:

a) contacting a test agent with the LGN/GPSM2 polypeptide or a fragment thereof;

b) detecting the binding between the polypeptide or fragment and the test agent; and

c) selecting the test agent that binds to the polypeptide as a candidate agent for treating or preventing breast cancer.

In the present invention, the therapeutic effect may be correlated with the binding level LGN/GPSM2 polypeptide or a functional fragment thereof. For example, when the test agent or compound bind to LGN/GPSM2 polypeptide or a functional fragment thereof, the test agent or compound may be identified or selected as the candidate agent or compound having the therapeutic effect. Alternatively, when the test agent or compound does not bind to LGN/GPSM2 polypeptide or a functional fragment thereof, the test agent or compound may be identified as the agent or compound having no significant therapeutic effect.

In one embodiment of the present invention, a fragment of LGN/GPSM2 polypeptide having a biological activity equivalent to the LGN/GPSM2 polypeptide may be used for the present screening method. In preferred embodiments, for example, the following activities or properties can be shown as the biological activity of LGN/GPSM2 polypeptide:

promoting activity of cell proliferation,

DNA synthesis enhancing activity,

binding activity to TRIOBP/tara or PBK/TOPK, and

PBK/TOPK-mediated phosphorylation.

That is, a fragment of LGN/GPSM2 polypeptide that has at least one activity among them may be referred to as a functional fragment. In more preferred embodiments, a functional fragment of LGN/GPSM2 polypeptide that has such activity(ies) can contain a TPR (Tetratricopeptide repeats) domain of LGN/GPSM2 polypeptide to retain or maintain the activity(ies). Specifically, for example, in order to retain or maintain the activity(ies), a TRP domain may be selected from the group consisting of;

amino acid residues 62-95 of SEQ ID NO: 40,

amino acid residues 102-135 of SEQ ID NO: 40,

amino acid residues 202-235 of SEQ ID NO: 40,

amino acid residues 242-275 of SEQ ID NO: 40,

amino acid residues 282-315 of SEQ ID NO: 40, and

amino acid residues 322-355 of SEQ ID NO: 40.

Likewise, in another embodiments, a functional fragment of LGN/GPSM2 polypeptide can also contain a GoLoco domain of LGN/GPSM2 polypeptide to retain or maintain the activity(ies). In more preferred embodiments, a functional fragment of LGN/GPSM2 polypeptide can contain at least one of the GoLoco domains selected from the group consisting of;

amino acid residues 489-511 of SEQ ID NO: 40,

amino acid residues 544-566 of SEQ ID NO: 40,

amino acid residues 594-616 of SEQ ID NO: 40, and

amino acid residues 628-650 of SEQ ID NO: 40.

In another embodiments, a functional fragment of LGN/GPSM2 polypeptide can also contain at least one of the TPR domains and at least one of the GoLoco domains of LGN/GPSM2 polypeptide. Accordingly, a functional fragment of LGN/GPSM2 polypeptide can contain;

(a) at least one of the TRP domains selected from the group consisting of;

amino acid residues 62-95 of SEQ ID NO: 40,

amino acid residues 102-135 of SEQ ID NO: 40,

amino acid residues 202-235 of SEQ ID NO: 40,

amino acid residues 242-275 of SEQ ID NO: 40,

amino acid residues 282-315 of SEQ ID NO: 40, and

amino acid residues 322-355 of SEQ ID NO: 40, and

(b) at least one of the GoLoco domains selected from the group consisting of;

amino acid residues 489-511 of SEQ ID NO: 40,

amino acid residues 544-566 of SEQ ID NO: 40,

amino acid residues 594-616 of SEQ ID NO: 40, and

amino acid residues 628-650 of SEQ ID NO: 40.

In the present invention, the functional fragment of LGN/GPSM2 polypeptide may consist of the amino acid sequence of less than about 600, 500, 400, 300, 200, 100, 50, or 30 contiguous residues selected from the amino sequence of SEQ ID NO:53 (677 amino acids residues). For example, preferable fragments contain any one domain to be required for retaining the activity, and consist of 25-200 or 25-100 contiguous residues selected from the amino sequence of SEQ ID NO:53 in length.

In addition, in preferred embodiments, the functional fragment may further contain at least one of phosphorylated sites of LGN/GPSM2 polypetide. For instance, it was revealed that the LGN/GPSM2 polypetide is phosphorylated at Ser401, Thr519 and/or Ser558 in the amino acid sequence of SEQ ID NO: 53. Accordingly, in the functional fragments, at least one amino acid residue corresponding to the position selected from Ser401, Thr519 and/or Ser558 of SEQ ID NO: 53 may be conserved to retain or maintain the activity(ies) of LGN/GPSM2 polypetide.

Alternatively, these phosphorylation sites correspond to Ser408, Thr526 and/or Ser565 in the amino acid sequence of SEQ ID NO:40. Accordingly, in the functional fragments, at least one amino acid residue corresponding to the position selected from Ser408, Thr526 and/or Ser565 of SEQ ID NO:40 may be conserved to retain or maintain the activity(ies) of the LGN/GPSM2 polypetide.

Therapeutic effect includes any of the following effect, such as inhibition of the growth of cancerous breast cells, involution or regression of a breast tumor, induction of remission and suppression of occurrence of breast cancer. Effectively treating breast cancer decreases mortality and improves the prognosis of individuals having breast cancer, decreases the levels of tumor markers in the blood, and alleviates detectable symptoms accompanying breast cancer.

The binding of a test agent to the LGN/GPSM2 polypeptide may be, for example, detected by immunoprecipitation using an antibody against the LGN/GPSM2 polypeptide. Therefore, for the purpose for such detection, it is preferred that the LGN/GPSM2 polypeptide or fragments thereof used for the screening contains an antibody recognition site. The antibody used for the screening may be one that recognizes an antigenic region (e.g., epitope) of the present LGN/GPSM2 polypeptide which preparation methods are well known in the art, and any method may be employed in the present invention to prepare such antibodies and equivalents thereof.

Alternatively, the LGN/GPSM2 polypeptide or a fragment thereof may be expressed as a fusion protein comprising at its N- or C-terminus a recognition site (epitope) of a monoclonal antibody, whose specificity has been revealed, to the N- or C-terminus of the polypeptide. Any commercially available epitope-antibody system can be used (Experimental Medicine 1995, 13:85-90). Vectors which can express a fusion protein with, for example, beta-galactosidase, maltose binding protein, glutathione S-transferase, green florescence protein (GFP), and such by the use of its multiple cloning sites are commercially available and can be used for the present invention. Furthermore, fusion proteins containing much smaller epitopes to be detected by immunoprecipitation with an antibody against the epitopes are also known in the art (Experimental Medicine 1995, 13:85-90). Such epitopes consisting of several to a dozen amino acids so as not to change the property of the LGN/GPSM2 polypeptide or fragments thereof can also be used in the present invention. Examples include polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage), and such.

Glutathione S-transferase (GST) is also well-known as the counterpart of the fusion protein to be detected by immunoprecipitation. When GST is used as the protein to be fused with the LGN/GPSM2 polypeptide or fragment thereof to form a fusion protein, the fusion protein can be detected either with an antibody against GST or a substance specifically binding to GST, i.e., such as glutathione (e.g., glutathione-Sepharose 4B).

In immunoprecipitation, an immune complex is formed by adding an antibody (recognizing the LGN/GPSM2 polypeptide or a fragment thereof itself, or an epitope tagged to the polypeptide or fragment) to the reaction mixture of the LGN/GPSM2 polypeptide and the test agent. If the test agent has the ability to bind the polypeptide, then the formed immune complex will consists of the LGN/GPSM2 polypeptide, the test agent, and the antibody. On the contrary, if the test agent is devoid of such ability, then the formed immune complex only consists of the LGN/GPSM2 polypeptide and the antibody. Therefore, the binding ability of a test agent to LGN/GPSM2 polypeptide can be examined by, for example, measuring the size of the formed immune complex. Any method for detecting the size of a substance can be used, including chromatography, electrophoresis, mass spectrometry, and such. For example, when mouse IgG antibody is used for the detection, Protein A or Protein G sepharose can be used for quantitating the formed immune complex.

For more details on immunoprecipitation see, for example, Harlow et al., Antibodies, Cold Spring Harbor Laboratory publications, New York, 1988, 511-52.

Furthermore, the LGN/GPSM2 polypeptide or a fragment thereof used for the screening of agents that bind to thereto may be bound to a carrier. Example of carriers that may be used for binding the polypeptides include insoluble polysaccharides, such as agarose, cellulose and dextran; and synthetic resins, such as polyacrylamide, polystyrene and silicon; preferably commercially available beads and plates (e.g., multi-well plates, biosensor chip, etc.) prepared from the above materials may be used. When using beads, they may be filled into a column. Alternatively, the use of magnetic beads is also known in the art, and enables to readily isolate polypeptides and agents bound on the beads via magnetism.

The binding of a polypeptide to a carrier may be conducted according to routine methods, such as chemical bonding and physical adsorption. Alternatively, a polypeptide may be bound to a carrier via antibodies specifically recognizing the protein. Moreover, binding of a polypeptide to a carrier can also be conducted by means of interacting molecules, such as the combination of avidin and biotin.

Screening using such carrier-bound LGN/GPSM2 polypeptide or fragments thereof include, for example, contacting a test agent to the carrier-bound polypeptide, incubating the mixture, washing the carrier, and detecting and/or measuring the agent bound to the carrier. The binding may be carried out in buffer, for example, but are not limited to, phosphate buffer and Tris buffer, as long as the buffer does not inhibit the binding.

A screening method wherein such carrier-bound LGN/GPSM2 polypeptide or fragments thereof and a composition (e.g., cell extracts, cell lysates, etc.) are used as the test agent, such method is generally called affinity chromatography. For example, the LGN/GPSM2 polypeptide may be immobilized on a carrier of an affinity column, and a test agent, containing a substance capable of binding to the polypeptides, is applied to the column. After loading the test agent, the column is washed, and then the substance bound to the polypeptide is eluted with an appropriate buffer.

A biosensor using the surface plasmon resonance phenomenon may be used as a mean for detecting or quantifying the bound agent in the present invention. When such a biosensor is used, the interaction between the LGN/GPSM2 polypeptide and a test agent can be observed real-time as a surface plasmon resonance signal, using only a minute amount of the polypeptide and without labeling (for example, BIAcore, Pharmacia). Therefore, one can evaluate the binding between the polypeptide and test agent using a biosensor such as BIAcore.

Methods of screening for molecules that bind to a specific protein among synthetic chemical compounds, or molecules in natural substance banks or a random phage peptide display library by exposing the specific protein immobilized on a carrier to the molecules, and methods of high-throughput screening based on combinatorial chemistry techniques (Wrighton et al., Science 1996, 273:458-64; Verdine, Nature 1996, 384:11-3) to isolate not only proteins but chemical compounds are also well-known to those skilled in the art. These methods can also be used for screening agents (including agonist and antagonist) that bind to the LGN/GPSM2 protein or fragments thereof.

When the test agent is a protein, for example, West-Western blotting analysis (Skolnik et al., Cell 1991, 65:83-90) can be used for the present method. Specifically, a protein binding to the LGN/GPSM2 polypeptide can be obtained by preparing first a cDNA library from cells, tissues, organs, or cultured cells (e.g., PC cell lines) expected to express at least one protein binding to the LGN/GPSM2 polypeptide using a phage vector (e.g., ZAP), expressing the proteins encoded by the vectors of the cDNA library on LB-agarose, fixing the expressed proteins on a filter, reacting the purified and labeled LGN/GPSM2 polypeptide with the above filter, and detecting the plaques expressing proteins to which the LGN/GPSM2 polypeptide has bound according to the label of the LGN/GPSM2 polypeptide.

Labeling substances such as radioisotope (e.g., ³H, ¹⁴C, ³²P, ³³P, ³⁵S, ¹²⁵I, ¹³¹I), enzymes (e.g., alkaline phosphatase, horseradish peroxidase, Beta-galactosidase, Beta-glucosidase), fluorescent substances (e.g., fluorescein isothiocyanate (FITC), rhodamine) and biotin/avidin, may be used for the labeling of LGN/GPSM2 polypeptide in the present method. When the protein is labeled with radioisotope, the detection or measurement can be carried out by liquid scintillation. Alternatively, when the protein is labeled with an enzyme, it can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Moreover, the LGN/GPSM2 polypeptide bound to the protein can be detected or measured by utilizing an antibody that specifically binds to the LGN/GPSM2 polypeptide, or a peptide or polypeptide (for example, GST) that is fused to the LGN/GPSM2 polypeptide. In case of using an antibody in the present screening, the antibody is preferably labeled with one of the labeling substances mentioned above, and detected or measured based on the labeling substance. Alternatively, the antibody against the LGN/GPSM2 polypeptide may be used as a primary antibody to be detected with a secondary antibody that is labeled with a labeling substance. Furthermore, the antibody bound to the LGN/GPSM2 polypeptide in the present screening may be detected or measured using protein G or protein A column.

Alternatively, in another embodiment of the screening method of the present invention, two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton et al., Cell 1992, 68:597-612” and “Fields et al., Trends Genet 1994, 10:286-92”). In two-hybrid system, LGN/GPSM2 polypeptide or a fragment thereof is fused to the SRF-binding region or GAL4-binding region and expressed in yeast cells. A cDNA library is prepared from cells expected to express at least one protein binding to the LGN/GPSM2 polypeptide, such that the library, when expressed, is fused to the VP16 or GAL4 transcriptional activation region. The cDNA library is then introduced into the above yeast cells and the cDNA derived from the library is isolated from the positive clones detected (when a protein binding to the LGN/GPSM2 polypeptide is expressed in the yeast cells, the binding of the two activates a reporter gene, making positive clones detectable). A protein encoded by the cDNA can be prepared by introducing the cDNA isolated above to E. coli and expressing the protein.

As a reporter gene, for example, Ade2 gene, lacZ gene, CAT gene, luciferase gene and such can be used in addition to the HIS3 gene.

The agent isolated by this screening is a candidate for agonists or antagonists of the LGN/GPSM2 polypeptide. The term “agonist” refers to molecules that activate the function of the polypeptide by binding thereto. On the other hand, the term “antagonist” refers to molecules that inhibit the function of the polypeptide by binding thereto. Moreover, an agent isolated by this screening as an antagonist is a candidate that inhibits the in vivo interaction of the LGN/GPSM2 polypeptide with molecules (including nucleic acids (RNAs and DNAs) and proteins (e.g., the substrate phosphorylated by the LGN/GPSM2 polypeptide)).

IV-1-2. Identifying Agents by Detecting Biological Activity of the LGN/GPSM2 Polypeptide

According to the present invention, the expression of LGN/GPSM2 gene was shown to be associated with the growth and/or survival of cancer cells, in particular, breast cancer cells. Therefore, agents that suppress or inhibit the biological function of the translational product of the LGN/GPSM2 gene are candidates for treating or preventing cancer. Thus, the present invention also provides a method for screening a compound for treating or preventing cancer, in particular, breast cancer, using the LGN/GPSM2 polypeptide or fragments thereof. Alternatively, a candidate compound suitable for the treatment and/or prevention of breast cancer may be identified by the present invention. Such methods include the steps of:

a) contacting a test compound with the LGN/GPSM2 polypeptide or a fragment thereof;

b) detecting the biological activity of the polypeptide or fragment of step (a).

c) comparing the biological activity of the polypeptide or fragment with the biological activity detected in the absence of the compound; and

d) selecting the compound that suppresses the biological activity of the polypeptide as a candidate compound for treating or preventing cancer. According to the present invention, the therapeutic effect of the test agent or compound on inhibiting the cell growth or a candidate agent or compound for treating or preventing LGN/GPSM2 associating disease, e.g., breast cancer, may be evaluated. Therefore, the present invention also provides a method of screening for a candidate agent or compound for inhibiting the cell growth or a candidate agent or compound for treating or preventing LGN/GPSM2 associating disease, e.g., breast cancer, using the LGN/GPSM2 polypeptide or fragments thereof including the steps as follows:

a) contacting a test agent or compound with the LGN/GPSM2 polypeptide or a functional fragment thereof; and

b) detecting the biological activity of the polypeptide or fragment of step (a), and

c) correlating the biological activity of b) with the therapeutic effect of the test agent or compound.

In the present invention, the therapeutic effect may be correlated with the biological activity LGN/GPSM2 polypeptide or a functional fragment thereof. For example, when the test agent or compound suppresses or inhibits the biological activity LGN/GPSM2 polypeptide or a functional fragment thereof as compared to a level detected in the absence of the test agent or compound, the test agent or compound may identified or selected as the candidate agent or compound having the therapeutic effect. Alternatively, when the test agent or compound does not suppress or inhibit the biological activity LGN/GPSM2 polypeptide or a functional fragment thereof as compared to a level detected in the absence of the test agent or compound, the test agent or compound may identified as the agent or compound having no significant therapeutic effect.

In preferred embodiments, biological activity of LGN/GPSM2 polypeptide is cell proliferative activity or DNA synthesis enhancing activity. The cell proliferative activity may be detected by obserbing proliferation of cell line. Meanwhile DNA synthesis enhancing activity can be evaluated by, for example, MTT and colony formation assays and BrdUrd-incorporation assays.

Any LGN/GPSM2 polypeptide or fragment thereof can be used for the screening so long as it has one biological activity of the LGN/GPSM2 polypeptide that can be used as an index in the present screening method. Any functional fragments or equivalents as described above, may be used for the the present screening method.

The present invention discloses that LGN/GPSM2 interacts with TRIOBP/tera or PBK/TOPK in breast cancer cells to promote cell growth or proliferation. Thus, the present invention provides methods of screening for a compound suitable for the treatment and/or prevention of cancer, in particular, breast cancer. Alternatively, a candidate compound suitable for the treatment and/or prevention of breast cancer may be identified by the present invention. Such methods include the steps of:

(a) contacting an TRIOBP/tera polypeptide or functional equivalent thereof with a LGN/GPSM2 polypeptide or functional equivalent thereof in the presence of a test compound;

(b) detecting the binding between the polypeptides of step (a); and

(c) selecting the test compound that inhibits the binding between the TRIOBP/tera and LGN/GPSM2 polypeptides; or

(a) contacting an PBK/TOPK polypeptide or functional equivalent thereof with a LGN/GPSM2 polypeptide or functional equivalent thereof in the presence of a test compound;

(b) detecting the binding between the polypeptides of step (a); and

(c) selecting the test compound that inhibits the binding between the PBK/TOPK and LGN/GPSM2 polypeptides.

In one embodiment of the present invention, a functional equivalent of LGN/GPSM2 polypeptide is referred to a polypeptide that has a biological activity equivalent to the LGN/GPSM2 polypeptide. In preferred embodiments, for example, following activities or properties can be shown as the biological activity of LGN/GPSM2 polypeptide: promoting activity of cell proliferation,

DNA synthesis enhancing activity,

binding activity to TRIOBP/tara or PBK/TOPK, and

PBK/TOPK-mediated phosphorylation.

Accordingly, above described functional fragment of LGN/GPSM2 polypeptide may also be functional equivalent of LGN/GPSM2 polypeptide.

According to the present invention, the therapeutic effect of the test agent or compound on inhibiting the cell growth or a candidate agent or compound for treating or preventing LGN/GPSM2 associating disease, e.g., breast cancer, may be evaluated. Therefore, the present invention also provides a method for screening a candidate agent or compound that suppresses the proliferation of breast cancer cells, and a method for screening a candidate agent or compound for treating or preventing breast cancer.

More specifically, the method includes the steps of:

(a) contacting a LGN/GPSM2 protein with a PBK/TOPK protein in the presence of an test agent or compound;

(b) detecting the level of binding between the LGN/GPSM2 and PBK/TOPK proteins;

(c) comparing the binding level of the LGN/GPSM2 and PBK/TOPK proteins with that detected in the absence of the test agent or compound; and

d) correlating the binding level of c) with the therapeutic effect of the test agent or compound.

In the present invention, the therapeutic effect may be correlated with the binding level of the LGN/GPSM2 and PBK/TOPK proteins. For example, when the test agent or compound reduces the binding level of LGN/GPSM2 and PBK/TOPK proteins as compared to a level detected in the absence of the test agent or compound, the test agent or compound may identified or selected as the candidate agent or compound having the therapeutic effect. Alternatively, when the test agent or compound does not reduce the binding level of LGN/GPSM2 and PBK/TOPK proteins as compared to a level detected in the absence of the test agent or compound, the test agent or compound may identified as the agent or compound having no significant therapeutic effect.

In the context of the present invention, a functional equivalent of a LGN/GPSM2, TRIOBP/tera or PBK/TOPK polypeptide is a polypeptide that has a biological activity equivalent to a LGN/GPSM2 polypeptide (SEQ ID NO: 40), TRIOBP/tera polypeptide (SEQ ID NO: 43) or PBK/TOPK polypeptide (SEQ ID NO: 45), respectively.

As a method of screening for compounds that inhibit the phosphorylation of LGN/GPSM2 by PBK/TOPK, any method known in the art can be used. For example, screening can be carried out using an in vitro assay system, such as a cellular system. The present invention involves identifying test compounds that regulate LGN/GPSM2-mediated phosphorylation of PBK/TOPK. Accordingly, the present invention provides a method of screening for compounds suitable for the treatment and/or prevention of cancer, in particular, breast cancer. Alternatively, a candidate compound suitable for the treatment and/or prevention of breast cancer may be identified by the present invention. Such methods including the steps of:

(a) incubating LGN/GPSM2 and PBK/TOPK in the presence of a test compound under conditions suitable for the phosphorylation of LGN/GPSM2 by PBK/TOPK, wherein the LGN/GPSM2 is a polypeptide selected from the group consisting of:

i. a polypeptide the amino acid sequence of SEQ ID NO: 40 (LGN/GPSM2);

ii. a polypeptide having the amino acid sequence of SEQ ID NO: 40 wherein one or more amino acids are substituted, deleted, or inserted, provided the polypeptide has a biological activity equivalent to the polypeptide consisting of the amino acid sequence of SEQ ID NO: 40;

iii. a polypeptide encoded by a polynucleotide that hybridizes under stringent conditions to a polynucleotide consisting of the nucleotide sequence of SEQ ID NO: 39 or 41, provided the polypeptide has a biological activity equivalent to a polypeptide consisting of the amino acid sequence of SEQ ID NO: 40, and

wherein the PBK/TOPK is a polypeptide selected from the group consisting of:

i. a polypeptide the amino acid sequence of SEQ ID NO: 45 (PBK/TOPK);

ii. a polypeptide having the amino acid sequence of SEQ ID NO: 45 wherein one or more amino acids are substituted, deleted, or inserted, provided the polypeptide has a biological activity equivalent to the polypeptide consisting of the amino acid sequence of SEQ ID NO: 45;

iii. a polypeptide encoded by a polynucleotide that hybridizes under stringent conditions to a polynucleotide consisting of the nucleotide sequence of SEQ ID NO: 44, provided the polypeptide has a biological activity equivalent to a polypeptide consisting of the amino acid sequence of SEQ ID NO: 45;

(b) detecting a phosphorylation level of the LGN/GPSM2;

(c) comparing the phosphorylation level of the LGN/GPSM2 to a control level; and

(d) selecting a compound that decreases the phosphorylation level of the LGN/GPSM2 as compared to the control level.

According to the present invention, the therapeutic effect of the test agent or compound on inhibiting the cell growth or a candidate agent or compound for treating or preventing LGN/GPSM2 associating disease may be evaluated. Therefore, the present invention also provides a method of screening for a candidate agent or compound for inhibiting the cell growth or a candidate agent or compound for treating or preventing LGN/GPSM2 associating disease, using the LGN/GPSM2 polypeptide or fragments thereof including the steps as follows:

(a) incubating LGN/GPSM2 and PBK/TOPK in the presence of a test compound under conditions suitable for the phosphorylation of LGN/GPSM2 by PBK/TOPK, wherein the LGN/GPSM2 is a polypeptide selected from the group consisting of:

i. a polypeptide the amino acid sequence of SEQ ID NO: 40 (LGN/GPSM2);

ii. a polypeptide having the amino acid sequence of SEQ ID NO: 40 wherein one or more amino acids are substituted, deleted, or inserted, provided the polypeptide has a biological activity equivalent to the polypeptide consisting of the amino acid sequence of SEQ ID NO: 40;

iii. a polypeptide encoded by a polynucleotide that hybridizes under stringent conditions to a polynucleotide consisting of the nucleotide sequence of SEQ ID NO: 39 or 41, provided the polypeptide has a biological activity equivalent to a polypeptide consisting of the amino acid sequence of SEQ ID NO: 40, and

wherein the PBK/TOPK is a polypeptide selected from the group consisting of:

i. a polypeptide the amino acid sequence of SEQ ID NO: 45 (PBK/TOPK);

ii. a polypeptide having the amino acid sequence of SEQ ID NO: 45 wherein one or more amino acids are substituted, deleted, or inserted, provided the polypeptide has a biological activity equivalent to the polypeptide consisting of the amino acid sequence of SEQ ID NO: 45;

iii. a polypeptide encoded by a polynucleotide that hybridizes under stringent conditions to a polynucleotide consisting of the nucleotide sequence of SEQ ID NO: 44, provided the polypeptide has a biological activity equivalent to a polypeptide consisting of the amino acid sequence of SEQ ID NO: 45;

(b) detecting a phosphorylation level of the LGN/GPSM2;

(c) comparing the phosphorylation level of the LGN/GPSM2 to a control level; and

(d) correlating the phosphorylation level of c) with the therapeutic effect of the test agent or compound.

In another embodiment of the present invention, the present invention also provides a method of screening for a candidate agent or compound for inhibiting the cell growth or a candidate agent or compound for treating or preventing an LGN/GPSM2-associated disease, using the LGN/GPSM2 polypeptide or fragments thereof including the steps as follows:

(a) contacting a LGN/GPSM2 polypeptide or a functional equivalent thereof with a protein kinase in the presence of a test compound under a suitable condition for phosphorylation;

(b) detecting the phosphorylation level of the LGN/GPSM2 polypeptide or functional equivalent thereof at one or two serine residues and/or a threonine residue corresponding to Ser401, Thr519 and/or Ser558 of SEQ ID NO: 53;

(c) comparing the phosphorylation level with the expression level or activity detected in the absence of the test compound; and

(d) selecting the test compound that reduces the phosphorylation level as a candidate compound for treating or preventing cancer.

Alternatively, these phosphorylation sites correspond to Ser408, Thr526 and/or Ser565 of SEQ ID NO:40. Accordingly, the present invention also provides a method of screening for a candidate agent or compound for inhibiting the cell growth or a candidate agent or compound for treating or preventing an LGN/GPSM2-associated disease, using the LGN/GPSM2 polypeptide or fragments thereof including the step of (b)′ detecting the phosphorylation level of the LGN/GPSM2 polypeptide or functional equivalent thereof at Ser408, Thr526 and/or Ser565 of SEQ ID NO:40.

In such embodiment of the present invention, a functional equivalent of LGN/GPSM2 polypeptide is referred to a polypeptide that has a biological activity equivalent to the LGN/GPSM2 polypeptide. In preferred embodiments, for example, following activities or properties can be shown as the biological activity of LGN/GPSM2 polypeptide:

promoting activity of cell proliferation, and

DNA synthesis enhancing activity.

Accordingly, above described functional fragment of LGN/GPSM2 polypeptide may also be functional equivalent of LGN/GPSM2 polypeptide as long as those activities are retained.

In the present invention, the therapeutic effect may be correlated with the phosphorylation level of the LGN/GPSM2 polypeptide or a functional fragment thereof. For example, when the test agent or compound suppresses or inhibits the phosphorylation level of the LGN/GPSM2 polypeptide or a functional fragment thereof as compared to a level detected in the absence of the test agent or compound, the test agent or compound may identified or selected as the candidate agent or compound having the therapeutic effect. Alternatively, when the test agent or compound does not suppress or inhibit the phosphorylation level of the LGN/GPSM2 polypeptide or a functional fragment thereof as compared to a level detected in the absence of the test agent or compound, the test agent or compound may identified as the agent or compound having no significant therapeutic effect.

In the context of the present invention, the conditions suitable for the phosphorylation of LGN/GPSM2 by PBK/TOPK may be provided with an incubation of LGN/GPSM2 and PBK/TOPK in the presence of a phosphate donor, e.g., ATP. The conditions suitable for the LGN/GPSM2 phosphorylation by PBK/TOPK also include culturing cells expressing the polypeptides. For example, such a cell may be a transformant cell harboring an expression vector containing a polynucleotide that encodes the LGN/GPSM2 polypeptide and/or the PBK/TOPK polypeptide. After the incubation, the phosphorylation level of the LGN/GPSM2 can be detected with a reagent, such as an antibody recognizing phosphorylated LGN/GPSM2.

Prior to the detection of phosphorylated LGN/GPSM2, LGN/GPSM2 may be separated from other elements, or cell lysate of LGN/GPSM2-expressing cells. For instance, gel electrophoresis may be used for the separation of LGN/GPSM2 from remaining components. Alternatively, LGN/GPSM2 may be captured by contacting LGN/GPSM2 with a carrier having an anti-LGN/GPSM2 antibody. When a labeled phosphate donor is used, the phosphorylation level of LGN/GPSM2 can be detected by tracing the label. For example, when radio-labeled ATP (e.g., ³²P-ATP) is used as a phosphate donor, radio activity of the separated LGN/GPSM2 correlates with the phosphorylation level of LGN/GPSM2. Alternatively, an antibody specifically recognizing phosphorylated LGN/GPSM2 from unphosphorylated LGN/GPSM2 may be used to detect phosphorylated LGN/GPSM2.

In some preferred embodiments, LGN/GPSM2 and PBK/TOPK may be incubated with a test agent under a condition suitable for the LGN/GPSM2 phoshorylation by PBK/TOPK. Such a condition may be provided by culturing cells expressing the polypeptides or lysate thereof. For example, such a cell may be a transformant cell harboring an expression vector containing a polynucleotide that encodes LGN/GPSM2 and/or PBK/TOPK. After the incubation with a test agent, the level of LGN/GPSM2 phoshorylation can be detected with an agent, such as an antibody recognizing the phosphorylation state of LGN/GPSM2. For instance, in the present invention, immunoassay or Western-blotting assay may be applied to the detection of the phosphorylation state of LGN/GPSM2.

In order to identify an agent that interferes with the LGN/GPSM2 phosphorylation by PBK/TOPK specifically, further screening may be performed, prior to or after the above-mentioned screening method. For example, by selecting an agent that binds to PBK/TOPK prior to or after the screening, a candidate agent that inhibits the function of PBK/TOPK may be identified. Such an agent may be selected by contacting a test agent with LGN/GPSM2 and PBK/TOPK, or fragment thereof; and identifying an agent that inhibits the level of the LGN/GPSM2 phosphorylation. Alternatively, it may also be confirmed whether a test agent affects the expression level of PBK/TOPK by determining the amount of the PBK/TOPK transcript or polypeptide.

Alternatively, other protein kinases may be used for phosphorylation of the LGN/GPSM2 polypeptide. According to the present invention, Ser401, Thr519 and Ser558 of SEQ ID NO: 53 are identified as phosphorylation sites of the LGN/GPSM2 polypeptide, and phosphorylation at these sites has been demonstrated to be involved in cell growth. Accordingly, agents or compounds that inhibit the phospholylation of the LGN/GPSM2 polypeptide at Ser401, Thr519 or Ser558 of SEQ ID NO: 53 may be useful for inhibiting cancer cell growth, therefore, treating or preventing cancer. Thus, the present invention also provides a method of screening for a candidate agent or compound for inhibiting the cancer cell growth or a candidate agent or compound for treating or preventing cancer, using the LGN/GPSM2 polypeptide or fragments thereof including the steps as follows:

(a) incubating a LGN/GPS2 polypeptide and a protein kinase in the presense of a test agent or compound under conditions suitable for the phosphorylation;

(b) detecting a phospholrylation level of the LGN/GPSM2 at Ser401, Thr519 and/or Ser558 of SEQ ID NO: 53;

(c) comparing the phosporylation level of the LGN/GPSM2 with that detected in the absence of the test agent or compound; and

(d) correlating the phosphorylation level of c) with the therapeutic effect of the test agent or compound.

Alternatively, these phosphorylation sites correspond to Ser408, Thr526 and/or Ser565 of SEQ ID NO:40. Accordingly, the present invention also provides a method of screening for a candidate agent or compound for inhibiting the cell growth or a candidate agent or compound for treating or preventing an LGN/GPSM2-associated disease, using the LGN/GPSM2 polypeptide or fragments thereof including the step of (b)′ detecting a phospholrylation level of the LGN/GPSM2 at Ser408, Thr526 and/or Ser565 of SEQ ID NO:40.

In the present invention, the therapeutic effect may be correlated with the phosphorylation level of the LGN/GPSM2 polypeptide or a functional fragment thereof at Ser401, Thr519 or Ser558 of SEQ ID NO: 53. For example, when the test agent or compound suppresses or inhibits the phosphorylation level of the LGN/GPSM2 polypeptide or a functional fragment thereof at Ser401, Thr519 and/or Ser558 of SEQ ID NO: 53 as compared to a level detected in the absence of the test agent or compound, the test agent or compound may identified or selected as the candidate agent or compound having the therapeutic effect. Alternatively, when the test agent or compound does not suppress or inhibit the phosphorylation level of the LGN/GPSM2 polypeptide or a functional fragment thereof as compared to a level detected in the absence of the test agent or compound, the test agent or compound may identified as the agent or compound having no significant therapeutic effect.

As a protein kinase, a serine-threonine kinases such as Aurora kinase may be preferably used in the present screening methods. Alternatively, a cell lysate or a whole cell, expressing the LGN/GPSM2 gene, may be incubated in the presence of a test agent or compound, and then the phosphorylation level in the lysate or cell may be detected. Such cell lysate or whole cell may be prepared from cancer cells such as breast cancer cells, or recombinat cells transfected with the LGN/GPSM2 gene.

The phosphorylation level of LGN/GPSM2 at Ser401, Thr519 and Ser558 may be detected antibodies specifically recognizing phospho-LGN/GPSM2 (Ser 401), phospho-LGN/GPSM2 (Thr519) and phospho-LGN/GPSM2 (Ser 558), respectively. When a whole cell is used in the present screening methods, the whole cell may be lysed using any conventional methods before detection of the phosphorylation level.

In the present invention, it is revealed that suppressing the phosphorylation of LGN/GPSM2 by PBK/TOPK, or binding between LGN/GPSM2 and PBK/TOPK, reduces breast cancer cell growth. Thus, by screening for candidate compounds that inhibits the binding or phosphorylation of LGN/GPSM2 by PBK/TOPK, candidate compounds that have the potential to treat or prevent breast cancers can be identified. The potential of these candidate compounds to treat or prevent breast cancers may be evaluated by second and/or further screening to identify therapeutic agent for breast cancers.

For example, when a compound that has a property selected from the group consisting of;

(a) a compound that binds to LGN/GPSM2 protein,

(b) a compound that reduces the biological activity of the polypeptide LGN/GPSM2,

(c) a compound that reduces the expression level LGN/GPSM2,

(d) a compound that reduces the expression level or activity of a reporter gene expressed under the control of the transcriptional regulatory region of the LGN/GPSM2 gene, and

(e) a compound that suppresses the phosphorylation level of a polypeptide comprising a PBK/TOPK-binding domain of a LGN/GPSM2 polypeptide,

(f) a compound that suppresses the phosphorylation level of LGN/GPSM2 polypeptide at Ser401, Thr519 and/or Ser558 of SEQ ID NO: 53.

wherein the compound breast cancer growth, it may be concluded that such compound has the LGN/GPSM2 specific therapeutic effect.

Preferably, the cell expressing LGN/GPSM2 and/or PBK/TOPK or functional equivalent thereof is a breast cancer cell.

In another aspect of the invention, a kit for screening for compounds suitable for the treatment and/or prevention cancer is also provided. The kit optionally includes the components of:

(a) a polypeptide selected from the group consisting of:

i. a polypeptide having the amino acid sequence of SEQ ID NO: 40 (LGN/GPSM2);

ii. a polypeptide having the amino acid sequence of SEQ ID NO: 40 wherein one or more amino acids are substituted, deleted, or inserted, provided the polypeptide has a biological activity equivalent to the polypeptide of the amino acid sequence of SEQ ID NO: 40; and

iii. a polypeptide encoded by a polynucleotide that hybridizes under stringent conditions to a polynucleotide of the nucleotide sequence of SEQ ID NO: 39 or 41 provided the polypeptide has a biological activity equivalent to a polypeptide of the amino acid sequence of SEQ ID NO: 40 and

(b) a polypeptide selected from the group consisting of:

i. a polypeptide having the amino acid sequence of SEQ ID NO: 45 (PBK/TOPK);

ii. a polypeptide having the amino acid sequence of SEQ ID NO: 45 wherein one or more amino acids are substituted, deleted, or inserted, provided the polypeptide has a biological activity equivalent to the polypeptide of the amino acid sequence of SEQ ID NO: 45; and

iii. a polypeptide encoded by a polynucleotide that hybridizes under stringent conditions to a polynucleotide of the nucleotide sequence of SEQ ID NO: 44, provided the polypeptide has a biological activity equivalent to a polypeptide of the amino acid sequence of SEQ ID NO: 45; and

(c) a reagent for detecting a phosphorylation level of LGN/GPSM2.

Further, this invention also provides a kit for screening for a compound suitable for the treatment and/or prevention cancer. The kit optionally includes the components of:

(a) a cell expressing a polypeptide selected from the group consisting of:

i. a polypeptide having the amino acid sequence of SEQ ID NO: 40 (LGN/GPSM2);

ii. a polypeptide having the amino acid sequence of SEQ ID NO: 40 wherein one or more amino acids are substituted, deleted, or inserted, provided the polypeptide has a biological activity equivalent to the polypeptide of the amino acid sequence of SEQ ID NO: 40

iii. a polypeptide encoded by a polynucleotide that hybridizes under stringent conditions to a polynucleotide of the nucleotide sequence of SEQ ID NO: 39 or 41, provided the polypeptide has a biological activity equivalent to a polypeptide of the amino acid sequence of SEQ ID NO: 40; and

(b) a reagent for detecting a phosphorylation level of LGN/GPSM2.

Furthermore, the kit for screening for compounds suitable for the treatment and/or prevention breast cancer may optionally include cells further expressing a polypeptide selected from the group consisting of:

i. a polypeptide having the amino acid sequence of SEQ ID NO: 45(PBK/TOPK);

ii. a polypeptide having the amino acid sequence of SEQ ID NO: 45 wherein one or more amino acids are substituted, deleted, or inserted, provided the polypeptide has a biological activity equivalent to the polypeptide of the amino acid sequence of SEQ ID NO: 45; and

iii. a polypeptide encoded by a polynucleotide that hybridizes under stringent conditions to a polynucleotide of the nucleotide sequence of SEQ ID NO: 44, provided the polypeptide has a biological activity equivalent to a polypeptide of the amino acid sequence of SEQ ID NO: 45.

In another aspect, the cell used in the kit is cancer cells, in particular, breast cancer.

In the present invention, the kit may further include a phosphate donor. The kit of the present invention may also include an antibody that recognizes a phosphorylation site(s) of a LGN/GPSM2 polypeptide or functional equivalent thereof as a reagent for detecting the phosphorylation level of LGN/GPSM2.

IV-2. Nucleotide Based Screening Methods

IV-2-1. Screening Method Using LGN/GPSM2 Gene

As discussed in detail above, by controlling the expression level of the LGN/GPSM2 gene, one can control the onset and progression of breast cancer. Thus, agents that may be used in the treatment or prevention of breast cancers can be identified through screenings that use the expression levels of LGN/GPSM2 gene as indices. In the context of the present invention, such screening may comprise, for example, the following steps:

a) contacting a test agent with a cell expressing the LGN/GPSM2 gene;

b) detecting the expression level of the LGN/GPSM2 gene;

c) comparing the expression level with the expression level detected in the absence of the agent; and

d) selecting the agent that reduces the expression level as a candidate agent for treating or preventing cancer.

According to the present invention, the therapeutic effect of the test agent or compound on inhibiting the cell growth or a candidate agent or compound for treating or preventing LGN/GPSM2 associating disease, e.g., breast cancer, may be evaluated. Therefore, the present invention also provides a method for screening a candidate agent or compound that suppresses the proliferation of breast cancer cells, and a method for screening a candidate agent or compound for treating or preventing LGN/GPSM2 associating disease.

In the context of the present invention, such screening may include, for example, the following steps:

a) contacting a test agent or compound with a cell expressing the LGN/GPSM2 gene;

b) detecting the expression level of the LGN/GPSM2 gene; and

c) correlating the expression level of b) with the therapeutic effect of the test agent or compound.

In the present invention, the therapeutic effect may be correlated with the expression level of the LGN/GPSM2 gene. For example, when the test agent or compound reduces the expression level of the LGN/GPSM2 gene as compared to a level detected in the absence of the test agent or compound, the test agent or compound may identified or selected as the candidate agent or compound having the therapeutic effect. Alternatively, when the test agent or compound does not reduce the expression level of the LGN/GPSM2 gene as compared to a level detected in the absence of the test agent or compound, the test agent or compound may identified as the agent or compound having no significant therapeutic effect.

An agent that inhibits the expression of the LGN/GPSM2 gene or the activity of its gene product can be identified by contacting a cell expressing the LGN/GPSM2 gene with a test agent and then determining the expression level of the LGN/GPSM2 gene. Naturally, the identification may also be performed using a population of cells that express the gene in place of a single cell. A decreased expression level detected in the presence of an agent as compared to the expression level in the absence of the agent indicates the agent as being an inhibitor of the LGN/GPSM2 gene, indicating that the agent is useful for inhibiting breast cancer, thus a candidate agent to be used for the treatment or prevention of breast cancer.

The expression level of a gene can be estimated by methods well known to one skilled in the art. The expression level of the LGN/GPSM2 gene can be, for example, determined using any method known in the art, including those described above under the item of ‘I-1. Method for diagnosing cancer or a predisposition for developing cancer’.

The cell or the cell population used for such identification may be any cell or any population of cells so long as it expresses the LGN/GPSM2 gene. For example, the cell or population may be or contain a breast epithelial cell derived from a tissue. Alternatively, the cell or population may be or contain an immortalized cell derived from a carcinoma cell. Cells expressing the LGN/GPSM2 gene include, for example, cell lines established from cancers (e.g., PC cell lines such as 22Rv1, C4-2B, P13, etc.).

Furthermore, the cell or population may be or contain a cell which has been transfected with the LGN/GPSM2 gene.

The present method allows screening of various agents mentioned above and is particularly suited for screening functional nucleic acid molecules including antisense RNA, siRNA, and such.

IV-2-2. Screening Method Using Transcriptional Regulatory Region of LGN/GPSM2 Gene

According to another aspect, the present invention provides a method which comprises the following steps of:

a) contacting a test agent with a cell into which a vector, comprising the transcriptional regulatory region of the LGN/GPSM2 gene and a reporter gene that is expressed under the control of the transcriptional regulatory region, has been introduced;

b) detecting the expression or activity of said reporter gene;

c) comparing the expression level or activity with the expression level or activity detected in the absence of the agent; and

d) selecting the agent that reduces the expression or activity of said reporter gene as a candidate agent for treating or preventing breast cancer.

According to the present invention, the therapeutic effect of the test agent or compound on inhibiting the cell growth or a candidate agent or compound for treating or preventing LGN/GPSM2 associating disease, e.g., breast cancer, may be evaluated. Therefore, the present invention also provides a method for screening a candidate agent or compound that suppresses the proliferation of breast cancer cells, and a method for screening a candidate agent or compound for treating or preventing LGN/GPSM2 associating disease.

According to another aspect, the present invention provides a method which includes the following steps of:

a) contacting a test agent or compound with a cell into which a vector, composed of the transcriptional regulatory region of the LGN/GPSM2 gene and a reporter gene that is expressed under the control of the transcriptional regulatory region, has been introduced;

b) detecting the expression or activity of said reporter gene; and

c) correlating the expression level of b) with the therapeutic effect of the test agent or compound.

In the present invention, the therapeutic effect may be correlated with the expression or activity of said reporter gene. For example, when the test agent or compound reduces the expression or activity of said reporter gene as compared to a level detected in the absence of the test agent or compound, the test agent or compound may identified or selected as the candidate agent or compound having the therapeutic effect. Alternatively, when the test agent or compound does not reduce the expression or activity of said reporter gene as compared to a level detected in the absence of the test agent or compound, the test agent or compound may identified as the agent or compound having no significant therapeutic effect.

Suitable reporter genes and host cells are well known in the art. The reporter construct required for the screening can be prepared using the transcriptional regulatory region of the LGN/GPSM2 gene, which can be obtained as a nucleotide segment containing the transcriptional regulatory region from a genome library based on the nucleotide sequence information of the gene.

The transcriptional regulatory region may be, for example, the promoter sequence of the LGN/GPSM2 gene. The reporter construct required for the screening can be prepared by connecting reporter gene sequence to the transcriptional regulatory region of LGN/GPSM2 gene. The transcriptional regulatory region of LGN/GPSM2 gene herein is the region from start codon to at least 500 bp upstream, preferably 1000 bp, more preferably 5000 or 10000 bp upstream. A nucleotide segment containing the transcriptional regulatory region can be isolated from a genome library or can be propagated by PCR. Methods for identifying a transcriptional regulatory region, and also assay protocol are well known (Molecular Cloning third edition chapter 17, 2001, Cold Springs Harbor Laboratory Press).

When a cell(s) transfected with a reporter gene that is operably linked to the regulatory sequence (e.g., promoter sequence) of the LGN/GPSM2 gene is used, an agent can be identified as inhibiting or enhancing the expression of the LGN/GPSM2 gene through detecting the expression level of the reporter gene product.

As a reporter gene, for example, Ade2 gene, lacZ gene, CAT gene, luciferase gene, HIS3 gene, and such well-known in the art can be used. Methods for detection of the expression of these genes are well known in the art.

IV-3. Selecting Therapeutic Agents that are Appropriate for a Particular Individual

Differences in the genetic makeup of individuals can result in differences in their relative abilities to metabolize various drugs. An agent that is metabolized in a subject to act as an anti-tumor agent can manifest itself by inducing a change in a gene expression pattern in the subject's cells from that characteristic of a cancerous state to a gene expression pattern characteristic of a non cancerous state. Accordingly, the LGN/GPSM2 gene differentially expressed between cancerous and non-cancerous breast tissue cells disclosed herein allow for a putative therapeutic or prophylactic inhibitor of breast cancer to be tested in a test cell population from a selected subject in order to determine if the agent is a suitable inhibitor of breast cancer in the subject.

To identify an inhibitor of breast cancer that is appropriate for a specific subject, a test cell population from the subject is exposed to a candidate therapeutic agent, and the expression of LGN/GPSM2 gene is determined.

In the context of the method of the present invention, test cell populations contain cancer cells expressing the LGN/GPSM2 gene. Preferably, the test cell is a breast epithelial cell.

Specifically, a test cell population may be incubated in the presence of a candidate therapeutic agent and the expression of the LGN/GPSM2 gene in the test cell population may be measured and compared to one or more reference profiles, e.g., a cancerous reference expression profile or a non-cancerous reference expression profile.

A decrease in the expression of the LGN/GPSM2 gene in a test cell population relative to a reference cell population containing cancer indicates that the agent has therapeutic potential. Alternatively, a similarity in the expression of the LGN/GPSM2 gene in a test cell population relative to a reference cell population not containing cancer indicates that the agent has therapeutic potential.

V. Pharmaceutical Compositions for Treating or Preventing Cancer:

The agents screened by any of the screening methods of the present invention, antisense nucleic acids and double-stranded molecules (e.g., siRNAs) of the LGN/GPSM2 gene, and antibodies against the LGN/GPSM2 polypeptide inhibit or suppress the expression of the LGN/GPSM2 gene, or the biological activity of the LGN/GPSM2 polypeptide and inhibit or disrupt breast cancer cell cycle regulation and breast cancer cell proliferation. Thus, the present invention provides compositions for treating or preventing breast cancer, which compositions include agents screened by any of the screening methods of the present invention, antisense nucleic acids and siRNAs of the LGN/GPSM2 gene, or antibodies against the LGN/GPSM2 polypeptide. The present compositions can be used for treating or preventing cancer, in particular, breast cancer.

The compositions may be used as pharmaceuticals for humans and other mammals, such as mice, rats, guinea-pigs, rabbits, cats, dogs, sheep, pigs, cattle, monkeys, baboons, and chimpanzees.

In the context of the present invention, suitable pharmaceutical formulations for the active ingredients of the present invention detailed below (including screened agents, antisense nucleic acids, double-stranded molecules (siRNA), antibodies, etc.) include those suitable for oral, rectal, nasal, topical (including buccal and sub-lingual), vaginal or parenteral (including intramuscular, subcutaneous and intravenous) administration, or for administration by inhalation or insufflation. Preferably, administration is intravenous. The formulations are optionally packaged in discrete dosage units.

Pharmaceutical formulations suitable for oral administration include capsules, microcapsules, cachets and tablets, each containing a predetermined amount of active ingredient. Suitable formulations also include powders, elixirs, granules, solutions, suspensions and emulsions. The active ingredient is optionally administered as a bolus electuary or paste. Alternatively, according to needs, the pharmaceutical composition may be administered non-orally, in the form of injections of sterile solutions or suspensions with water or any other pharmaceutically acceptable liquid. For example, the active ingredients of the present invention can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredient contained in such a preparation makes a suitable dosage within the indicated range acquirable.

Examples of additives that can be admixed into tablets and capsules include, but are not limited to, binders, such as gelatin, corn starch, tragacanth gum and arabic gum; excipients, such as crystalline cellulose; swelling agents, such as corn starch, gelatin and alginic acid; lubricants, such as magnesium stearate; sweeteners, such as sucrose, lactose or saccharin; and flavoring agents, such as peppermint, Gaultheria adenothrix oil and cherry. A tablet may be made by compression or molding, optionally with one or more formulational ingredients. Compressed tablets may be prepared by compressing in a suitable machine the active ingredients in a free-flowing form such as powder or granules, optionally mixed with a binder, lubricant, inert diluent, lubricating, surface active or dispersing agent. Molded tablets may be made via molding in a suitable machine a mixture of the powdered compound moistened with an inert liquid diluent. The tablets may be coated according to methods well known in the art. The tablets may optionally be formulated so as to provide slow or controlled release of the active ingredient in vivo. A package of tablets may contain one tablet to be taken on each of the month.

Furthermore, when the unit-dosage form is a capsule, a liquid carrier, such as oil, can be further included in addition to the above ingredients.

Oral fluid preparations may be in the form of, for example, aqueous or oily suspensions, solutions, emulsions, syrups or elixirs, or may be presented as a dry product for reconstitution with water or other suitable vehicle prior to use. Such liquid preparations may contain conventional additives such as suspending agents, emulsifying agents, non-aqueous vehicles (which may include edible oils) or preservatives.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Moreover, sterile composites for injection can be formulated following normal drug implementations using vehicles, such as distilled water, suitable for injection. Physiological saline, glucose, and other isotonic liquids, including adjuvants, such as D-sorbitol, D-mannose, D-mannitol, and sodium chloride, can be used as aqueous solutions for injection. These can be used in conjunction with suitable solubilizers, such as alcohol, for example, ethanol; polyalcohols, such as propylene glycol and polyethylene glycol; and non-ionic surfactants, such as Polysorbate 80™ and HCO-50.

Sesame oil or soy-bean oil can be used as an oleaginous liquid, which may be used in conjunction with benzyl benzoate or benzyl alcohol as a solubilizer, and may be formulated with a buffer, such as phosphate buffer and sodium acetate buffer; a pain-killer, such as procaine hydrochloride; a stabilizer, such as benzyl alcohol and phenol; and/or an anti-oxidant. A prepared injection may be filled into a suitable ampoule.

Formulations for rectal administration include suppositories with standard carriers such as cocoa butter or polyethylene glycol. Formulations for topical administration in the mouth, for example, buccally or sublingually, include lozenges, which contain the active ingredient in a flavored base such as sucrose and acacia or tragacanth, and pastilles comprising the active ingredient in a base such as gelatin, glycerin, sucrose or acacia. For intra-nasal administration of an active ingredient, a liquid spray or dispersible powder or in the form of drops may be used. Drops may be formulated with an aqueous or non-aqueous base also comprising one or more dispersing agents, solubilizing agents or suspending agents.

For administration by inhalation the compositions are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichiorotetrafluoroethane, carbon dioxide or other suitable gas. In the case of a pressurized aerosol, the dosage unit may be determined by providing a valve to deliver a metered amount.

Alternatively, for administration by inhalation or insufflation, the compositions may take the form of a dry powder composition, for example, a powder mix of an active ingredient and a suitable powder base such as lactose or starch. The powder composition may be presented in unit dosage form in, for example, capsules, cartridges, gelatin or blister packs from which the powder may be administered with the aid of an inhalator or insufflators.

Other formulations include implantable devices and adhesive patches; which release a therapeutic agent.

When desired, the above-described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited under the item of ‘V. Method for treating or preventing cancer’ (infra), of each of the active ingredients of the present invention or an appropriate fraction thereof.

V-1. Pharmaceutical Compositions Containing Screened Agents

The present invention provides compositions for treating or preventing cancers comprising any of the agents selected by the above-described screening methods of the present invention.

An agent screened by the method of the present invention can be directly administered or can be formulated into a dosage form according to any conventional pharmaceutical preparation method detailed above.

V-2. Pharmaceutical Compositions Comprising Double-Stranded Molecules

A double-stranded molecules against the LGN/GPSM2 gene (hereinafter, also referred to as ‘LGN/GPSM2 siRNA’) can be used to reduce the expression level of the gene. The phrase “double-stranded molecule” is the same meaning defined in the item ‘Definitions’.

Herein, the term “siRNA” refers to a double-stranded RNA molecule which prevents translation of a target mRNA as defined in the item ‘Definitions’. In the context of the present invention, the siRNA comprises a sense nucleic acid sequence and an antisense nucleic acid sequence against the up-regulated marker gene, LGN/GPSM2. The siRNA is constructed so that it both comprises a portion of the sense and complementary antisense sequences of the target gene (i.e., LGN/GPSM2 gene), and may also be a single construct taking a hairpin structure, wherein the sense and antisense strands are linked via a single-strand. The siRNA may either be a dsRNA or shRNA. A double-stranded molecule against the LGN/GPSM2 gene hybridizes to target mRNA, i.e., associates with the normally single-stranded mRNA transcript and thereby interfering with translation of the mRNA, which finally decreases or inhibits production (expression) of the polypeptide encoded by the gene. Thus, a double-stranded molecule of the invention can be defined by its ability to specifically hybridize to the mRNA of the LGN/GPSM2 gene under stringent conditions. Herein, the portion of the double-stranded molecule that hybridizes with the target mRNA is referred to as “target sequence” or “target nucleic acid” or “target nucleotide”.

In the context of the present invention, the target sequence of a double-stranded molecule is preferably less than 500, 200, 100, 50, or 25 base pairs in length. More preferably, the target sequence of a double-stranded molecule is 19-25 base pairs in length. Exemplary target nucleic acid sequences of LGN/GPSM2 siRNA includes the nucleotide sequences of SEQ ID NO:20 or 21. The nucleotide “t” in the sequence should be replaced with “u” in RNA or derivatives thereof. Accordingly, for example, the present pharmaceutical composition may comprise a double-stranded RNA molecule (siRNA) comprising the nucleotide sequence

5′-GCAUGAGAGAAGACCAUUC-3′ (for SEQ ID NO: 20) or

5′-GGACGUGCCUUUGGAAAUC-3′ (for SEQ ID NO:21) as the sense strand.

In order to enhance the inhibition activity of the double-stranded molecule, several nucleotides can be added to the 3′end of the target sequence in the sense and/or antisense strand. The number of nucleotides to be added is at least 2, generally 2 to 10, preferably 2 to 5. The added nucleotides form a single strand at the 3′end of the sense and/or antisense strand of the double-stranded molecule, which are referred as to “3′-overhang”. The preferred examples of nucleotides to be added include “t” and “u”, but are not limited to. In cases where double-stranded molecules consists of a single polynucleotide to form a hairpin loop structure, a 3′ overhang sequence may be added to the 3′ end of the single polynucleotide. Although the double-stranded molecule is an siRNA, 3′-overhangs may be replaced by deoxyribonucleotides (Elbashir S M et al., Genes Dev 2001 Jan. 15, 15(2): 188-200).

A loop sequence consisting of an arbitrary nucleotide sequence can be located between the sense and antisense strands in order to form a hairpin loop structure. Thus, the double stranded molecule contained in the composition of the present invention may take the general formula 5′-[A]-[B]-[A′]-3′, wherein [A] is a polynucleotide strand which comprises the sense strand sequence of a target sequence specifically hybridizing to an mRNA or a cDNA of the LGN/GPSM2 gene. Herein, the polynucleotide strand which comprises the sense strand sequence of a target sequence specifically hybridizing to an mRNA or a cDNA of the LGN/GPSM2 gene may be referred to as “sense strand”. In preferred embodiments, [A] is the sense strand; [B] is a single stranded polynucleotide consisting of 3 to 23 nucleotides; and [A′] is a polynucleotide strand which comprises the antisense strand sequence of a target sequence specifically hybridizing to an mRNA or a cDNA of the LGN/GPSM2 gene (i.e., a sequence hybridizing to the target sequence of the sense strand [A]). Herein, the polynucleotide strand which comprises the antisense strand sequence of a target sequence specifically hybridizing to an mRNA or a cDNA of the LGN/GPSM2 gene may be referred to as “antisense strand”. The region [A] hybridizes to [A′], and then a loop consisting of the region [B] is formed. The loop sequence may be preferably 3 to 23 nucleotides in length. The loop sequence, for example, can be selected from a group consisting of following sequences (www.ambion.com/techlib/tb/tb_{—}506.html):
CCC, CCACC, or CCACACC: Jacque J M et al., Nature 2002, 418: 435-8.
UUCG: Lee N S et al., Nature Biotechnology 2002, 20:500-5; Fruscoloni P et al., Proc Natl Acad Sci USA 2003, 100(4):1639-44.
UUCAAGAGA: Dykxhoom D M et al., Nature Reviews Molecular Cell Biology 2003, 4:457-67.
‘UUCAAGAGA (“ttcaagaga” in DNA)’ is a particularly suitable loop sequence. Furthermore, loop sequence consisting of 23 nucleotides also provides an active siRNA (Jacque J M et al., Nature 2002, 418:435-8).
Exemplary hairpin siRNA suitable for use in the context of the present invention include, for LGN/GPSM2-siRNA,
5′-GCAUGAGAGAAGACCAUUC-[b]-GAAUGGUCUUCUCUCAUGC-3′ (target sequence of SEQ ID NO:20); and
5′-GGACGUGCCUUUGGAAAUC-[b]-GAUUUCCAAAGGCACGUCC-3′ (target sequence of SEQ ID NO:21).

Other nucleotide sequences of suitable siRNAs for the present invention can be designed using an siRNA design computer program available from the Ambion website (www.ambion.com/techlib/misc/siRNA_finder.html). The computer program selects nucleotide sequences for siRNA synthesis based on the following protocol.

Selection of siRNA Target Sites:

1. Beginning with the AUG start codon of the object transcript, scan downstream for AA dinucleotide sequences. Record the occurrence of each AA and the 3′ adjacent 19 nucleotides as potential siRNA target sites. Tuschl et al. Genes Cev 1999, 13(24):3191-7 don't recommend designing siRNA to the 5′ and 3′ untranslated regions (UTRs) and regions near the start codon (within 75 nucleotides) as these may be richer in regulatory protein binding sites. UTR-binding proteins and/or translation initiation complexes may interfere with binding of the siRNA endonuclease complex.

2. Compare the potential target sites to the human genome database and eliminate from consideration any target sequences with significant homology to other coding sequences. The homology search can be performed using BLAST (Altschul S F et al., Nucleic Acids Res 1997, 25:3389-402; J Mol Biol 1990, 215:403-10.), which can be found on the NCBI server at: www.ncbi.nlm.nih.gov/BLAST/.

3. Select qualifying target sequences for synthesis. At Ambion, preferably several target sequences can be selected along the length of the gene to evaluate. Standard techniques are known in the art for introducing double-stranded molecule into cells. For example, an double-stranded molcule can be directly introduced into the cells in a form that is capable of binding to the mRNA transcripts. In these embodiments, the double-stranded molecules are typically modified as described above for antisense molecules. Other modifications are also available, for example, cholesterol-conjugated siRNAs have shown improved pharmacological properties (Song et al., Nature Med 2003, 9:347-51). These conventionally used techniques may also be applied for the double-stranded molecule contained in the present compositions.

Alternatively, a DNA encoding the double-stranded molecule may be carried in a vector (hereinafter, also referred to as ‘double-stranded molecule vector’) and the double-stranded molecule may be contained in the present composition in the form of vector which enables expression of the double-stranded molecule in vivo. Such vectors may be produced, for example, by cloning a portion of the target LGN/GPSM2 gene sequence sufficient to inhibit the in vivo expression of the LGN/GPSM2 gene into an expression vector having operatively-linked regulatory sequences (e.g., a RNA polymerase III transcription unit from the small nuclear RNA (snRNA) U6 or the human H1 RNA promoter) flanking the sequence in a manner that allows for expression (by transcription of the DNA molecule) of both strands (Lee N S et al., Nature Biotechnology 2002, 20: 500-5). For example, an RNA molecule that is antisense to mRNA of the LGN/GPSM2 gene is transcribed by a first promoter (e.g., a promoter sequence 3′ of the cloned DNA) and an RNA molecule that is the sense strand for the mRNA of the LGN/GPSM2 gene is transcribed by a second promoter (e.g., a promoter sequence 5′ of the cloned DNA). The sense and antisense strands hybridize in vivo to generate an siRNA construct for silencing the expression of the LGN/GPSM2 gene. Alternatively, the sense and antisense strands may be transcribed together with the help of one promoter. In this case, the sense and antisense strands may be linked via a polynucleotide sequence to form a single-stranded siRNA construct having secondary structure, e.g., hairpin.

Thus, the present pharmaceutical composition for treating or preventing cancer, including breast cancer and hepatocellular carcinoma, comprises at least any one of the double-stranded molecule and a vector expressing thereof in vivo. In U.S. Pat. No. 7,345,156, it is disclosed that antisense S-oligonucleotides of LGN/GPSM suppresses growth of human hepatoma SNU475 cells. Therefore, the double-stranded molecule of the present invention is useful for treating or preventing cancer, including breast cancer and hepatocellular carcinoma.

For introducing the double-stranded molecule or vector into the cell, transfection-enhancing agent can be used. FuGENE6 (Roche diagnostics), Lipofectamine 2000 (Invitrogen), Oligofectamine (Invitrogen), and Nucleofector (Wako pure Chemical) are useful as the transfection-enhancing agent. Therefore, the present pharmaceutical composition may further include such transfection-enhancing agents.

In the present invention, the double-stranded molecule can be administered to the subject either as a naked nucleic acids, in conjunction with a delivery reagent, or as a recombinant plasmid or viral vector which expresses the double-stranded molecule.

Suitable delivery reagents for administration in conjunction with the present double-stranded molecule include the Minis Transit TKO lipophilic reagent; lipofectin; lipofectamine; cellfectin; or polycations (e.g., polylysine), or liposomes. A preferred delivery reagent is a liposome.

Liposomes can aid in the delivery of the double-stranded molecule to a particular tissue, such as retinal or tumor tissue, and can also increase the blood half-life of the inhibitory nucleic acids. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present double-stranded molecules comprises a ligand molecule that can deliver the liposome to the cancer site. Ligands which bind to receptors prevalent in tumor cells, such as monoclonal antibodies that bind to tumor antigens, are preferred.

Particularly preferably, the liposomes encapsulating the present double-stranded molecules are modified so as to avoid clearance by the mononuclear macrophage and reticuloendothelial systems, for example, by having opsonization-inhibition moieties bound to the surface of the structure. In one embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present inhibitory nucleic acids to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH. sub. 3 and a solvent mixture such as tetrahydrofuran and water in a 30:12 ratio at 60. degree. C. Vectors expressing inhibitory nucleic acids of the invention are discussed above. Such vectors expressing at least one inhibitory nucleic acids of the invention can also be administered directly or in conjunction with a suitable delivery reagent, including the Minis Transit LT1 lipophilic reagent; lipofectin; lipofectamine; cellfectin; polycations (e.g., polylysine) or liposomes. Methods for delivering recombinant viral vectors, which express inhibitory nucleic acids of the invention, to an area of cancer in a patient are within the skill of the art.

The double-stranded molecules of the present invention can be administered to the subject by any means suitable for delivering the double-stranded molecule into cancer sites. For example, the double-stranded molecules can be administered by gene gun, electroporation, or by other suitable parenteral or enteral administration routes. Suitable enteral administration routes include oral, rectal, or intranasal delivery.

Suitable parenteral administration routes include intravascular administration (e.g., intravenous bolus injection, intravenous infusion, intra-arterial bolus injection, intraarterial infusion and catheter instillation into the vasculature); peri- and intra-tissue injection (e.g., peri-tumoral and intra-tumoral injection, intra-retinal injection, or subretinal injection); subcutaneous injection or deposition including subcutaneous infusion (such as by osmotic pumps); direct application to the area at or near the site of cancer, for example by a catheter or other placement device (e.g., a retinal pellet or a suppository or an implant comprising a porous, non-porous, or gelatinous material); and inhalation. It is preferred that injections or infusions of the double-stranded molecules or vector be given at or near the site of cancer.

The inhibitory nucleic acids of the invention can be administered in a single dose or in multiple doses. Where the administration of the double-stranded molecules of the invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the double-stranded molecules of the invention to a given subject. For example, the double-stranded molecules can be administered to the subject once, for example, as a single injection or deposition at or near the cancer site. Alternatively, the double-stranded molecules can be administered once or twice daily to a subject for a period of from about three to about twenty-eight days, more preferably from about seven to about ten days. In a preferred dosage regimen, the double-stranded molecules are injected at or near the site of cancer once a day for seven days. Where a dosage regimen comprises multiple administrations, it is understood that the effective amount of an double-stranded molecules administered to the subject can comprise the total amount of an double-stranded molecules administered over the entire dosage regimen.

V-3. Pharmaceutical Compositions Comprising Antisense Nucleic Acids

Antisense nucleic acids targeting the LGN/GPSM2 gene can be used to reduce the expression level of the gene, which is up-regulated in cancerous cells, including breast cancer cells. Such antisense nucleic acids are useful for the treatment of cancer, in particular breast cancer, and thus are also encompassed by the present invention. An antisense nucleic acid acts by binding to the nucleotide sequence of the LGN/GPSM2 gene, or mRNAs corresponding thereto, thereby inhibiting the transcription or translation of the gene, promoting the degradation of the mRNAs, and/or inhibiting the expression of the protein encoded by the gene.

Thus, as a result, an antisense nucleic acid inhibits the LGN/GPSM2 protein to function in the cancerous cell. Herein, the phrase “antisense nucleic acids” refers to nucleotides that specifically hybridize to a target sequence and includes not only nucleotides that are entirely complementary to the target sequence but also that comprise mismatches of one or more nucleotides. For example, the antisense nucleic acids of the present invention include polynucleotides that have a homology of at least 70% or higher, preferably of at least 80% or higher, more preferably of at least 90% or higher, even more preferably of at least 95% or higher over a span of at least 15 continuous nucleotides of the LGN/GPSM2 gene or the complementary sequence thereof. Algorithms known in the art can be used to determine such homology.

Antisense nucleic acids of the present invention act on cells producing proteins encoded by the LGN/GPSM2 gene by binding to the DNA or mRNA of the gene, inhibiting their transcription or translation, promoting the degradation of the mRNA, and inhibiting the expression of the protein, finally inhibiting the protein to function.

Antisense nucleic acids of the present invention can be made into an external preparation, such as a liniment or a poultice, by admixing it with a suitable base material which is inactive against the nucleic acids.

Also, as needed, the antisense nucleic acids of the present invention can be formulated into tablets, powders, granules, capsules, liposome capsules, injections, solutions, nose-drops and freeze-drying agents by adding excipients, isotonic agents, solubilizers, stabilizers, preservatives, pain-killers, and such. An antisense-mounting medium can also be used to increase durability and membrane-permeability. Examples include, but are not limited to, liposomes, poly-L-lysine, lipids, cholesterol, lipofectin, or derivatives of these. These can be prepared by following known methods.

The antisense nucleic acids of the present invention inhibit the expression of the LGN/GPSM2 protein and are useful for suppressing the biological activity of the protein. In addition, expression-inhibitors, comprising antisense nucleic acids of the present invention, are useful in that they can inhibit the biological activity of the LGN/GPSM2 protein.

The antisense nucleic acids of present invention also include modified oligonucleotides. For example, thioated oligonucleotides may be used to confer nuclease resistance to an oligonucleotide.

V-4. Pharmaceutical Compositions Comprising Antibodies

The function of a gene product of the LGN/GPSM2 gene which is over-expressed in cancers, in particular breast cancer can be inhibited by administering a compound that binds to or otherwise inhibits the function of the LGN/GPSM2 gene products. An antibody against the LGN/GPSM2 polypeptide is such a compound and can be used as the active ingredient of a pharmaceutical composition for treating or preventing breast cancer.

The present invention relates to the use of antibodies against a protein encoded by the LGN/GPSM2 gene, or fragments of the antibodies. As used herein, the term “antibody” refers to an immunoglobulin molecule having a specific structure, that interacts (i.e., binds) only with the antigen that was used for synthesizing the antibody (i.e., the gene product of an up-regulated marker) or with an antigen closely related thereto. Molecules comprising the antigen that was used for synthesizing the antibody and molecules comprising the epitope of the antigen recognized by the antibody can be mentioned as closely related antigens thereto.

Furthermore, an antibody used in the present pharmaceutical compositions may be a fragment of an antibody or a modified antibody, so long as it binds to the protein encoded by the LGN/GPSM2 gene (e.g., an immunologically active fragment of anti-LGN/GPSM2 antibody). For instance, the antibody fragment may be Fab, F(ab′)₂, Fv, or single chain Fv (scFv), in which Fv fragments from H and L chains are ligated by an appropriate linker (Huston J S et al., Proc Natl Acad Sci USA 1988, 85:5879-83). Such antibody fragments may be generated by treating an antibody with an enzyme, such as papain or pepsin. Alternatively, a gene encoding the antibody fragment may be constructed, inserted into an expression vector, and expressed in an appropriate host cell (see, for example, Co M S et al., J Immunol 1994, 152:2968-76; Better M et al., Methods Enzymol 1989, 178:476-96; Pluckthun A et al., Methods Enzymol 1989, 178:497-515; Lamoyi E, Methods Enzymol 1986, 121:652-63; Rousseaux J et al., Methods Enzymol 1986, 121:663-9; Bird R E et al., Trends Biotechnol 1991, 9:132-7).

An antibody may be modified by conjugation with a variety of molecules, such as polyethylene glycol (PEG). The present invention includes such modified antibodies. The modified antibody can be obtained by chemically modifying an antibody. Such modification methods are conventional in the field.

Alternatively, the antibody used for the present invention may be a chimeric antibody having a variable region derived from a non-human antibody against the LGN/GPSM2 polypeptide and a constant region derived from a human antibody, or a humanized antibody, comprising a complementarity determining region (CDR) derived from a non-human antibody, a frame work region (FR) and a constant region derived from a human antibody. Such antibodies can be prepared by using known technologies. Humanization can be performed by substituting rodent CDRs or CDR sequences for the corresponding sequences of a human antibody (see e.g., Verhoeyen et al., Science 1988, 239:1534-6). Accordingly, such humanized antibodies are chimeric antibodies, wherein substantially less than an intact human variable domain has been substituted by the corresponding sequence from a non-human species.

Complete human antibodies comprising human variable regions in addition to human framework and constant regions can also be used. Such antibodies can be produced using various techniques known in the art. For example in vitro methods involve use of recombinant libraries of human antibody fragments displayed on bacteriophage (e.g., Hoogenboom et al., J Mol Biol 1992, 227:381-8). Similarly, human antibodies can be made by introducing human immunoglobulin loci into transgenic animals, e.g., mice in which the endogenous immunoglobulin genes have been partially or completely inactivated. This approach is described, e.g., in U.S. Pat. Nos. 6,150,584, 5,545,807; 5,545,806; 5,569,825; 5,625,126; 5,633,425; and 5,661,016.

When the obtained antibody is to be administered to the human body (antibody treatment), a human antibody or a humanized antibody is preferable for reducing immunogenicity.

Antibodies obtained as above may be purified to homogeneity. For example, the separation and purification of the antibody can be performed according to separation and purification methods used for general proteins. For example, the antibody may be separated and isolated by the appropriately selected and combined use of column chromatographies, such as affinity chromatography, filter, ultrafiltration, salting-out, dialysis, SDS polyacrylamide gel electrophoresis, isoelectric focusing, and others (Antibodies: A Laboratory Manual. Ed Harlow and David Lane, Cold Spring Harbor Laboratory (1988)), but are not limited thereto. A protein A column and protein G column can be used as the affinity column. Exemplary protein A columns to be used include, for example, Hyper D, POROS, and Sepharose F.F. (Pharmacia).

Exemplary chromatography, with the exception of affinity includes, for example, ion-exchange chromatography, hydrophobic chromatography, gel filtration, reverse-phase chromatography, adsorption chromatography, and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

VI. Methods for Treating or Preventing Cancer:

Cancer therapies directed at specific molecular alterations that occur in cancer cells have been validated through clinical development and regulatory approval of antitumor pharmaceuticals such as trastuzumab (Herceptin) for the treatment of advanced cancers, imatinib mesylate (Gleevec) for chronic myeloid leukemia, gefitinib (Iressa) for non-small cell lung cancer (NSCLC), and rituximab (anti-CD20 mAb) for B-cell lymphoma and mantle cell lymphoma (Ciardiello F et al., Clin Cancer Res 2001, 7:2958-70, Review; Slamon D J et al., N Engl J Med 2001, 344:783-92; Rehwald U et al., Blood 2003, 101:420-4; Fang G et al., Blood 2000, 96:2246-53). These drugs are clinically effective and better tolerated than traditional anti-tumor agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L, Oncology 2002, 63 Suppl 1:47-56; Klejman A et al., Oncogene 2002, 21:5868-76). Therefore, future cancer treatments will involve combining conventional drugs with target-specific agents aimed at different characteristics of tumor cells such as angiogenesis and invasiveness.

These modulatory methods can be performed ex vivo or in vitro (e.g., by culturing the cell with the agent) or, alternatively, in vivo (e.g., by administering the agent to a subject). The methods involve administering a protein or combination of proteins or a nucleic acid molecule or combination of nucleic acid molecules as therapy to counteract aberrant expression of the differentially expressed genes or aberrant activity of their gene products.

Diseases and disorders that are characterized by increased (relative to a subject not suffering from the disease or disorder) expression levels or biological activities of LGN/GPSM2 genes and gene products, respectively, may be treated with therapeutics that antagonize (i.e., reduce or inhibit) activity of the over-expressed gene. Therapeutics that antagonize activity can be administered therapeutically or prophylactically.

Accordingly, therapeutics that may be utilized in the context of the present invention include, e.g., (i) a polypeptide of the over-expressed LGN/GPSM2 gene or analogs, derivatives, fragments or homologs thereof; (ii) antibodies against the over-expressed gene or gene products; (iii) nucleic acids encoding the over-expressed gene; (iv) antisense nucleic acids or nucleic acids that are “dysfunctional” (i.e., due to a heterologous insertion within the nucleic acids of over-expressed gene); (v) double-stranded molecule (e.g., siRNA); or (vi) modulators (i.e., inhibitors, antagonists that alter the interaction between an over-expressed polypeptide and its binding partner). The dysfunctional antisense molecules are utilized to “knockout” endogenous function of a polypeptide by homologous recombination (see, e.g., Capecchi, Science 1989, 244: 1288 92).

Increased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well known within the art include, but are not limited to, immunoassays (e.g., by Western blot analysis, immunoprecipitation followed by sodium dodecyl sulfate (SDS) polyacrylamide gel elec trophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods of the present invention may include the step of contacting a cell with an agent that modulates one or more of the activities of the LGN/GPSM2 gene products. Examples of agent that modulates protein activity include, but are not limited to, nucleic acids, proteins, naturally occurring cognate ligands of such proteins, peptides, peptidomimetics, and other small molecule.

Thus, the present invention provides methods for treating or alleviating a symptom of breast cancer, or preventing breast cancer in a subject by decreasing the expression of the LGN/GPSM2 gene or the activity of the gene product. The present method is particularly suited for treating or preventing breast cancer expressing LGN/GPSM2 including breast carcinoma. In the present invention, it was confirmed that siRNA against the LGN/GPSM gene, which is up-regulated in breast cancer, suppresses the growth of the breast cancer cells. Therefore, the double-stranded molecule against the LGN/GPSM gene is useful for treating breast cancer. In addition, U.S. Pat. No. 7,345,156 discloses that antisense S-oligonucleotides of LGN/GPSM suppresses growth of human hepatoma SNU475 cells. Therefore, the siRNA against the LGN/GPSM gene is also useful for treating hepatocellular carcinoma.

Suitable therapeutics can be administered prophylactically or therapeutically to a subject suffering from or at risk of (or susceptible to) developing a breast cancer. Such subjects can be identified by using standard clinical methods or by detecting an aberrant expression level (“up-regulation” or “over-expression”) of the LGN/GPSM2 gene or aberrant activity of the gene product.

According to an aspect of the present invention, an agent screened through the present method may be used for treating or preventing breast cancer. Methods well known to those skilled in the art may be used to administer the agents to patients, for example, as an intraarterial, intravenous, or percutaneous injection or as an intranasal, transbronchial, intramuscular, or oral administration. If said agent is encodable by a DNA, the DNA can be inserted into a vector for gene therapy and the vector administered to a patient to perform the therapy.

The dosage and methods for administration vary according to the body-weight, age, sex, symptom, condition of the patient to be treated and the administration method; however, one skilled in the art can routinely select suitable dosage and administration method.

For example, although the dose of an agent that binds to the LGN/GPSM2 polypeptide and regulates the activity of the polypeptide depends on the aforementioned various factors, the dose is generally about 0.1 mg to about 100 mg per day, preferably about 1.0 mg to about 50 mg per day and more preferably about 1.0 mg to about 20 mg per day, when administered orally to a normal adult human (60 kg weight).

When administering the agent parenterally, in the form of an injection to a normal adult human (60 kg weight), although there are some differences according to the patient, target organ, symptoms and methods for administration, it is convenient to intravenously inject a dose of about 0.01 mg to about 30 mg per day, preferably about 0.1 to about 20 mg per day and more preferably about 0.1 to about 10 mg per day. In the case of other animals, the appropriate dosage amount may be routinely calculated by converting to 60 kg of body-weight.

Similarly, a pharmaceutical composition of the present invention may be used for treating or preventing breast cancer. Methods well known to those skilled in the art may be used to administer the compositions to patients, for example, as an intraarterial, intravenous, or percutaneous injection or as an intranasal, transbronchial, intramuscular, or oral administration.

For each of the aforementioned conditions, the compositions, e.g., polypeptides and organic compounds, can be administered orally or via injection at a dose ranging from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age, body weight and sex of the subject, the precise disorder being treated, and its severity. Also the route of administration may vary depending upon the condition and its severity. In any event, appropriate and optimum dosages may be routinely calculated by those skilled in the art, taking into consideration the above-mentioned factors.

In particular, an siRNA against the LGN/GPSM2 gene can be given to the patient by direct application onto the ailing site or by injection into a blood vessel so that it will reach the site of ailment.

The dosage of the siRNA of the present invention can be adjusted suitably according to the patient's condition and used in desired amounts. For example, a dose range of 0.1 to 100 mg/kg, preferably 0.1 to 50 mg/kg can be administered.

VII. Double-Stranded Molecules and Vectors Encoding Them

According to the present invention, siRNA comprising either of the sequences of SEQ ID NOs: 20 and 21 was demonstrated to suppress cell growth or viability of cells expressing the LGN/GPSM2 gene. Therefore, double-stranded molecules comprising any of these sequences and vectors expressing the molecules are considered to serve as preferable pharmaceutics for treating or preventing diseases which involve the proliferation of LGN/GPSM2 gene expressing cells (e.g., breast cancer). Thus, according to an aspect, the present invention provides double-stranded molecules comprising a sequence selected from the group of SEQ ID NOs: 20and 21, and a vector, or vectors expressing the molecules. More specifically, the present invention provides a double-stranded molecule, when introduced into a cell expressing the LGN/GPSM2 gene, inhibits expression of the gene, which molecule comprises a sense strand and an antisense strand, wherein the sense strand comprises a nucleotide sequence selected from the group consisting of SEQ ID NOs: 20 and 21 as the target sequence, and the antisense strand comprises a nucleotide sequence complementary to the target sequence of the sense strand so that the sense and antisense strands hybridize to each other to form the double-stranded molecule.

The target sequence comprised in the sense strand may consist of a sequence of a portion of SEQ ID NO: 39 or 41 that is less than about 500, 400, 300, 200, 100, 75, 50 or 25 contiguous nucleotides. For example, the target sequence may be from about 19 to about 25 contiguous nucleotides from the nucleotide sequence of SEQ ID NO: 39or 41.

Accordingly, the present invention provides the double-stranded molecules comprising a sense strand and an antisense strand, wherein the sense strand comprises a nucleotide sequence corresponding to a target sequence. In preferable embodiments, the sense strand hybridizes with antisense strand at the target sequence to form the double-stranded molecule having between 19 and 25 nucleotide pair in length.

The present invention is not limited thereto, but suitable target sequences include the sequences of SEQ ID NOs: 20 and 21.

The double-stranded molecule of the present invention may be composed of two polynucleotide constructs, i.e., a polynucleotide comprising the sense strand and a polynucleotide comprising the antisense strand. Alternatively, the molecule may be composed of one polynucleotide construct; i.e., a polynucleotide comprising both the sense strand and the antisense strand, wherein the sense and antisense strands are linked via a single-stranded polynucleotide which enables hybridization of the target sequences within the sense and antisense strands by forming a hairpin structure. Herein, the single-stranded polynucleotide may also be referred to as “loop sequence” or “single-strand”. The single-stranded polynucleotide linking the sense and antisense strands may consist of 3 to 23 nucleotides. See under the item of “IV-2. Pharmaceutical compositions comprising double-stranded molecules” for more details on the double-stranded molecules of the present invention.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include phosphorothioate linkages, 2′-0-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxyabasic residue incorporation (US20060122137).

In another embodiment, modifications can be used to enhance the stability or to increase targeting efficiency of the double-stranded molecule. Modifications include chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deaza, 7-alkyl, or 7-alkenyl purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the invention may comprise both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule consisting of a DNA strand (polynucleotide) and an RNA strand (polynucleotide), a chimera type double-stranded molecule comprising both DNA and RNA on any or both of the single strands (polynucleotides), or the like may be formed for enhancing stability of the double-stranded molecule. The hybrid of a DNA strand and an RNA strand may be the hybrid in which either the sense strand is DNA and the antisense strand is RNA, or the opposite so long as it has an activity to inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene.

In order to enhance stability of the double-stranded molecule, the molecule preferably contains as much DNA as possible, whereas to induce inhibition of the target gene expression, the molecule is required to be RNA within a range to induce sufficient inhibition of the expression. As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. That is, in preferable embodiments, a region flanking to the 3′-end of the antisense strand, or both of a region flanking to the 5′-end of sense strand and a region flanking to the 3′-end of antisense strand consists of RNA. For instance, the chimera or hybrid type double-stranded molecule of the present invention comprise following combinations.

sense strand:

5′-[- - - DNA - - - ]-3′

3′-(RNA)-[DNA]-5′

:antisense strand,

sense strand:

5′-(RNA)-[DNA]-3′

3′-(RNA)-[DNA]-5′

:antisense strand, and

sense strand:

5′-(RNA)-[DNA]-3′

3′-( - - - RNA - - - )-5′

:antisense strand.

The upstream partial region preferably is a domain consisting of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA comprises the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

Also included in the invention is a vector containing one or more of the double-stranded nucleic acid molecules described herein, and a cell containing the vector. A vector of the present invention preferably encodes a double-stranded nucleic acid molecule of the present invention in an expressible form. Herein, the phrase “in an expressible form” indicates that the vector, when introduced into a cell, will express the molecule. In a preferred embodiment, the vector includes regulatory elements necessary for expression of the double-stranded nucleic acid molecule. Such vectors of the present invention may be used for producing the present double-stranded nucleic acid molecules, or directly as an active ingredient for treating cancer. Specifically, the present invention provides a vector comprising each or both of a combination of polynucleotide comprising a sense strand nucleic acid and an antisense strand nucleic acid, wherein said sense strand nucleic acid comprises nucleotide sequence of SEQ ID NOs: 20 or 21, and wherein the antisense strand comprises a nucleotide sequence which is complementary to said sense strand, wherein the transcripts of said sense strand and said antisense strand hybridize to each other to form said double-stranded molecule, and wherein said vector, when introduced into a cell expressing the LGN/GPSM2 gene, inhibits expression of said gene.

Alternatively, the present invention provides vectors comprising each of a combination of polynucleotide comprising a sense strand nucleic acid and an antisense strand nucleic acid, wherein said sense strand nucleic acid comprises nucleotide sequence of SEQ ID NOs: 20 or 21, and said antisense strand nucleic acid consists of a sequence complementary to the sense strand, wherein the transcripts of said sense strand and said antisense strand hybridize to each other to form a double-stranded molecule, and wherein said vectors, when introduced into a cell expressing the LGN/GPSM2 gene, inhibits expression of said gene. Preferably, the polynucleotide is an oligonucleotide of between about 19 and 25 nucleotides in length (e.g., contiguous nucleotides from the nucleotide sequence of SEQ ID NO: 39 or 41). More preferably, the combination of polynucleotide comprises a single nucleotide transcript comprising the sense strand and the antisense strand linked via a single-stranded nucleotide sequence. More preferably, the combination of polynucleotide has the general formula 5′-[A]-[B]-[A′]-3′, wherein [A] is a nucleotide sequence comprising SEQ ID NO: 20 or 21; [B] is a nucleotide sequence consisting of about 3 to about 23 nucleotide; and [A′] is a nucleotide sequence complementary to [A].

Vectors of the present invention can be produced, for example, by cloning a LGN/GPSM2 sequence into an expression vector so that regulatory sequences are operatively-linked to the LGN/GPSM2 sequence in a manner to allow expression (by transcription of the DNA molecule) of both strands (Lee NS et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded nucleic acid molecule constructs for silencing of the gene. Alternatively, two vectors construct respectively encoding the sense and antisense strands of the double-stranded nucleic acid molecule are utilized to respectively express the sense and anti-sense strands and then forming a double-stranded nucleic acid molecule construct. Furthermore, the cloned sequence may encode a construct having a secondary structure (e.g., hairpin); namely, a single transcript of a vector contains both the sense and complementary antisense sequences of the target gene.

The vectors of the present invention may also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687). The vectors of the present invention may be, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded nucleic acid molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded nucleic acid molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

Hereinafter, the present invention is described in more detail with reference to the Examples. However, the following materials, methods and examples only illustrate aspects of the invention and in no way are intended to limit the scope of the present invention. As such, methods and materials similar or equivalent to those described herein can be used in the practice or testing of the present invention.

### EXAMPLS

I. Materials and Methods

1. Cell Lines and Breast Cancer Clinical Samples

Human breast cancer cell lines, HCC1937, MCF-7, MDA-MB-231, SK-BR-3, T47D, YMB-1, BT20, BT474, HBL100, HCC1395, MDA-MB-157, HCC1599, ZR-75-1, HCC1143, HCC1500, MDA-MB-453 and OCUB-F and human embryonic kidney, HEK293 cell were purchased from American Type Culture Collection (ATCC, Rockville, Md., USA). HMEC was purchased from Cambrex Bio Science Walkersville Inc. (CAMBREX, Walkersville, Md., USA). HBC4, HBC5 and BSY-1 cell lines were kindly provided from Dr. Takao Yamori of Division of Molecular Pharmacology, Cancer Chemotherapy Center, Japanese Foundation for Cancer Research. All cells were cultured according to previous reports (Park J H et al., Cancer Res. 2006, 66:9186-9195, Lin M L et al., Breast Cancer Res. 2007, 9:R17, Shimo A et al., Cancer cells. 2007,98:174-181). Tissue samples from surgically resected breast cancers and their corresponding clinical information were obtained from Department of Breast Surgery, Cancer Institute Hospital, Tokyo after obtaining written informed consent.

2. Semi-Quantitative RT-PCR

Total RNAs were extracted from each of microdissected breast cancer clinical samples, microdissected normal breast ductal cells and breast cancer cell lines using Rneasy Mini kits (Qiagen, Valencia, Calif., USA), and poly(A)⁺ RNAs isolated from mammary gland purchased from Takara Clontech (Kyoto, Japan) as described previously (Nishidate et al., Int J Oncol 2004, 25:797-819). Subsequently, T7-based amplification and reverse transcription were carried out as described previously (Nishidate et al., Int J Oncol 2004, 25:797-819). Appropriate dilutions of each single-stranded cDNA was prepared for subsequent PCR by monitoring Beta-actin as a quantitative control. The specific primer sequences are as follows: 5′-GGCACGTAAGTAACACTTCCTGG-3′ (SEQ ID NO: 1) and 5′-GTTACAGGCACTTACGGGAACC-3′ (SEQ ID NO: 2) for Hs.659320, 5′-CCAGTTGGGCAATGCTTATT-3′ (SEQ ID NO: 3) and 5′-CTCTTGCTTCTCCCACCTTG-3′ (SEQ ID NO: 4) for LGN/GPSM2, 5′-TTAGCTGTGCTCGCGCTACT-3′ (SEQ ID NO: 5) and 5′-TCACATGGTTCACACGGCAG-3′ (SEQ ID NO: 6) for Beta 2-microglobulin (Beta 2MG), 5′-GAGGTGATAGCATTGCTTTCG-3′ (SEQ ID NO: 7) and 5′-CAAGTCAGTGTACAGGTAAGC-3′ (SEQ ID NO: 8) for Beta-actin. Centre

3. Isolation and DNA Sequencing of cDNA

Among genes that were overexpressed in the majority of the invasive breast carcinoma examined on a cDNA microarray, one clone FLJ20046 (UniGene Accesson No. Hs.659320 (SEQ ID NO: 38)) was focused. In order to obtain a full-length cDNA of the transcript, 5′-RACE (rapid amplification of cDNA ends)-PCR was performed using SMART RACE cDNA amplification kit (Clontech) according to the supplier's recommendations. The cDNA template was synthesized from breast cancer cell line, MDA-MB-231 mRNA using oligo dT primer and an adaptor sequence, SMARTIIA oligo (5′-AAGCAGTGGTATCAACGCAGAGTACGCGGG-3′ (SEQ ID NO: 9)). RACE PCR was performed with universal primer mix (long primer; 5′-CTAATACGACTCACTATAGGGCAAGCAGTGGTATCAACGCAGAGT-3′ (SEQ ID NO: 10) and short primer; 5′-CTAATACGACTCACTATAGGGC-3′ (SEQ ID NO: 11)) and gene specific primer (5′-AACTGCAGACAGGACATCAGTCAGCA-3′ (SEQ ID NO: 12)) by 25 cycles of 94 degrees C. for 5sec, 68 degrees C. for 10 sec and 72 degrees C. for 3 min. PCR products were subjected to nested PCR using nested universal primer (5′-AAGCAGTGGTATCAACGCAGAGT-3′ (SEQ ID NO: 13)) and nested gene specific primer (5′-GCAGACTTCACAGACATCAGGTGTCC-3′ (SEQ ID NO: 14)) by 20 cycles of 94 degrees C. for 5 sec, 68 degrees C. for 10 sec and 72 degrees C. for 3 min. Nested PCR product was gel-extracted, cloned into pCR2.1 vector (Invitrogen) and sequenced. DNA sequences were confirmed by DNA sequencing (ABI3700, PE Applied Biosystems, Foster, Calif.). Obtained sequences were used as a query sequence to screen the BLAST database (http://www.ncbi.nlm.nih.gov).

4. Northern Blot Analysis

Northern blot membrane for breast cancer cell lines was prepared as described previously (Park J H et al., Cancer Res. 2006 66:9186-9195). Human multiple-tissue northern blots (Takara Clontech) were hybridized with [alpha-³²P]-dCTP-labeled specific probes prepared by RT-PCR (see below). Prehybridization, hybridization and washing were performed according to the supplier's recommendations. The blots were autoradiographed with intensifying screens at −80 degrees C. for 7 days. Specific probes for GPSM2 were prepared by RT-PCR using the following primer sets: 5′-GGCACGTAAGTAACACTTCCTGG-3′ (SEQ ID NO: 15) and 5′-GTTACAGGCACTTACGGGAACC-3′ (SEQ ID NO: 16) were for Hs.659320 (Probe1), 5′-GGCCATTGATTATCATCTGAAGC-3′ (SEQ ID NO: 17) and 5′-TCCTTACCGTGTTTGAAAGGAA-3′ (SEQ ID NO: 18) were for LGN/GPSM2 coding region (Ex8-15).

5. Plasmids and Oligonucleotide siRNA

Plasmids expressing siRNAs specific to LGN/GPSM2 were prepared by cloning the double-stranded oligonucleotides into psiU6BX3.0 vector (Shimokawa T et al., Cancer Res. 2003 63:6116-6120). The target sequences of the oligonucleotides for siRNA are as follows: 5′-GCGCGCTTTGTAGGATTCG-3′ (SEQ ID NO: 19) for control SCR (chroloplast Euglena gracilis gene coding for 5S and 16S rRNAs), 5′-GCATGAGAGAAGACCATTC-3′ (SEQ ID NO: 20) for si #1, 5′-GGACGTGCCTTTGGAAATC-3′ (SEQ ID NO: 21) for si #2, 5′-TCATGCGAGC AGACCATTC-3′ (SEQ ID NO: 22) for si #1-mm (underlines indicate mismatch sequence) and 5′-TCAACATGAGGAGACAGTC-3′ (SEQ ID NO: 23) for si #1-scramble. Complementary oligonucleotides were each phosphorylated by incubation with T4-polynucleotide kinase at 37 degrees C. for 30 min, followed by boiling and then slow cooling to room temperature to anneal the two oligonucleotides. Each product was ligated into psiU6BX3.0 to construct LGN/GPSM2-siRNA expression vectors. The gene-silencing effect of each vector was verified by semi-quantitative RT-PCR using GPSM2/LGN-specific primer, 5′-CCAGTTGGGCAATGCTTATT-3′ (SEQ ID NO: 24) and 5′-CTCTTGCTTCTCCCACCTTG-3′ (SEQ ID NO: 25).

To construct GPSM2 expression vectors, the entire coding sequence of GPSM2 cDNA was amplified by PCR using following primers; 5′-ATGCATGCCTCGAG TTATGAGAGAAGACCATTCTTTTCATG-3′ (SEQ ID NO: 26) and 5′-ACGTACGTGACTCGAGCTAATGGTCTGCCGATTTTTTCCC-3′ (SEQ ID NO: 27) (underlines indicate restriction enzyme sites). PCR product was inserted into the XhoI sites of pCAGGSnHA expression vector in frame with N-terminal HA tag. To construct GPSM2 expression vector controlled by Tet-Off inducible system, HA-tagged GPSM2 was PCR-amplified using following primers; 5′-ATGCATGC GCTAGCAAGCATGTACCCATACGATGTTCCAGATTACGCTGGAGGAGGAG GAAGAGAAGACCATTCTTTTCATGTT-3′ (SEQ ID NO: 28), 5′-ATGCATGC GATATCCTAATGGTCTGCCGATTTTTTCC-3′ (SEQ ID NO: 29) and pCAGGSnHA-GPSM2 as a template. PCR product was inserted into the NheI and EcoRV site of pTRE2 vector (Clontech).

For construction of full-length GPSM2 expression vector in E. coli, the entire coding sequence of GPSM2 was PCR-amplified using following primers; 5′-ATGCATGC CATATGAGAGAAGACCATTCTTTTCATGTTC-3′ (SEQ ID NO: 30) and 5′-ACGTACGTGACTCGAGATGGTCTGCCGATTTTTTCCCTGA-3′ (SEQ ID NO: 31), and the PCR product was inserted into the NdeI and XhoI site of pET28a vector (Novagen).

For construction of TRIOBP expression vectors, the entire coding sequence of TRIOBP isoform1 was PCR-amplified using following primers; 5′-ATGCATGC GAATTCGGCGGATGGAAGGGGCCGG-3′ (SEQ ID NO: 32) and 5′-ATGCATGC CTCGAGCTACTCAGCCAGGCTGTTGCG-3′ (SEQ ID NO: 33) and the PCR product was inserted into the pCAGGSn3F vector in frame with N-terminal 3xFLAG tag. PBK/TOPK expression vector was generated by Dr. J. H.-Park (Park J H et al., Cancer Res. 2006 66:9186-9195). DNA sequences of all constructs were confirmed by DNA sequencing (ABI3700, PE Applied Biosystems, Foster, Calif.).

siRNA oligonucleotides (Sigma Aldrich Japan KK, Tokyo, Japan) was used to further verify the knockdown effects of LGN/GPSM2 on cell cycle and proliferation. The sequences targeting each gene were as follows: 5′-GAAGCAGCAC-GACUUCUUC-3′ (SEQ ID NO: 34) (sense) and 5′-GAAGAAGUCGUGCUGCUUC-3′ (SEQ ID NO: 35) (antisense) for siEGFP (control), 5′-GGACGUGCCUUUGGAAAUC-3′ (SEQ ID NO: 36) (sense) and 5′-GAUUUCCAAAGGCACGUCC-3′ (SEQ ID NO: 37) (antisense) for siLGN/GPSM2. The sequences of siRNA targeting PBK/TOPK is described previously (Park J H et al., Cancer Res. 2006 66:9186-9195).

6. Western Blot Analysis

Cells were lysed with RIPA buffer (20 mM Tris-HCl, 150 mM NaCl, 1% Nonidet P-40, 0.5% deoxycholate, 0.1% SDS, 1 mM sodium fluoride, 1 mM sodium orthovanadate, pH8.0) containing 0.1% protease inhibitor cocktail III (Calbiochem, San Diego, Calif., USA). After homogenization, the cell lysates were incubated on ice for 30 minutes and centrifuged at 14,000 rpm for 15 minutes to separate the supernatant from the cell debris. The amount of total protein was estimated by protein assay kit (Bio-Rad, Hercules, Calif.), and then the cell lysates were mixed with SDS-sample buffer and boiled for 3 minutes before loading into SDS-polyacrylamide gels (Bio-Rad). After electrophoresis, the proteins were blotted onto nitrocellulose membrane (GE Healthcare, Buckinghamshire, United Kingdom). After blocking with 4% BlockAce blocking solution (Dainippon Pharmaceutical. Co., Ltd, Osaka, Japan), membranes were incubated with the primary antibodies as describe below. Finally, the membrane was incubated with HRP conjugated-secondary antibody (1:10000 dilution; GE Healthcare), and proteins were visualized by the ECL detection reagent (GE Healthcare). Beta-actin was used as a loading control. The primary antibodies used are as follows; Beta-actin (1:30000 dilution; clone AC-15, Sigma-Aldrich), anti-GPSM2 rabbit polyclonal antibody (1:500 dilution; ProteinTech, Chicago, Ill., USA).

7. Fluorescent-Activated Cell Sorting Analysis

T47D cells were synchronized their cell cycle by two different ways of treatments as follows; to synchronize the cell cycle from G1 phase, cells were treated with 1 mcg/ml of aphidicolin (Sigma-Aldrich) for 16 hours. Subsequently, the cells were collected every 3 hours up to 24 hours. To synchronize the cell cycle from mitotic phase, the cells were incubated with 0.3 mcg/ml nocodazole (Sigma-Aldrich) for 18 hours followed by gentle shaking-off the less-attached mitotic cells. Harvested cells were reseeded onto the plate and collected at each time point (0, 0.5, 1, 1.5, 2, 4 and 6 hours). The cells were fixed with 70% ethanol at −20 degrees C. overnight. Then, the cells were incubated with 1 mg/ml RNase A at 37 degrees C for 30minutes and stained with 50 mcg/ml propidium iodide (PI). The DNA content of the cell at each time points were analyzed by FACSCalibur (Becton Dickinson, Franklin Lakes, N.J., USA).

8.Lambda-Protein Phosphatase Assay

Cells were lysed with lysis buffer (50 mM Tris-HCl, 0.5% Igepal, 0.5% TritonX-100, 2% glycerol, 150 mM NaCl, 0.2% protease inhibitor cocktail Set III (Calbiochem), pH7.4). Aliquots of 10 mcg protein were supplemented with 2 mM MnCl₂, incubated with 800 units of lambda-protein phosphatase (New England Biolabs, Beverly, Mass.) or 25 mM of sodium fluoride for 60 minutes at 30 degrees C. Incubation was terminated by addition of SDS sample buffer and the samples were boiled for 3 minutes, subjected to western blot analysis according to the method described in western blot analysis section.

9. Cell Transfection and Treatments

T47D and HEK293 cells were transfected with expression vector constructs using FuGENE 6 transfection regent (Roche) according to the manufacturer's reccomendation. BT20 cells were trabsfected with expression vector using Lipofectamine2000 (Invitrogen). T47D cells were transfected with siRNA oligonucleotides using Lipofectamine RNAiMAX reagent (Invitrogen) according to the manufacturer's protocol. For establishment of cell lines that stably overexpress HA-LGN/GPSM2 under the control of Tet-Off-system, pTRE2-HA-LGN/GPSM2 was transfected into MCF7 Tet-Off cells (Invitrogen), using Lipofectamine2000 (Invitrogen). Transfected cells were incubated in the culture medium containing 0.4 mg/ml of hygromycin (Sigma) and 1 mcg/ml of Doxycuclin. Three weeks later, 120 individual colonies were selected by limiting dilution and screened for HA-LGN/GPSM2-stably-overexpressing clones. The expression of HA-LGN/GPSM2 was induced by the incubation in the Doxycyclin-free media for five days and the expression level of HA-LGN/GPSM2 in each clone was examined by western blot and immunocytochemical staining analyses using anti-HA monoclonal antibody (Roche).

10.Cell-Growth Assays

T47D and BT20 cells transfected with psiU6 plasmids were maintained in media containing appropriate concentrations of Geneticin. Cell viability was measured by MTT assay 10 days later, using Cell-counting kit8 (Dojindo). For colony-formation assays, the cells were fixed with 4% paraformaldehyde and stained with Giemsa solution 14 days after Geneticin selection. Cell growth of COS-7 cells transfected with pCAGGSnHA-LGN/GPSM2 vectors were measured by MTT assay 4 to 5 days after transfection.

11.Immunocytochemical Staining

T47D cells were fixed with phosphate-buffered saline (PBS) (−)-containing 4% paraformaldehyde for 15 minutes at room temperature, and rendered permeable with PBS(−)-containing 0.1% TritonX-100 at room temperature for 2 minutes. Subsequently, the cells were covered with 3% bovine serum albumin in PBS(−) for 1 hour at room temperature to block non-specific hybridization, followed by incubation with anti-GPSM2 rabbit polyclonal antibody (Proteintech) at 1:100 dilutions and anti-alpha-tubulin mouse monoclonal antibody (T6199: Sigma) at 1:100 dilutions for 1 hour at room temperature. After washing with PBS(−), cells were stained by A exa594-conjugated anti-rabbit secondary antibody and Alexa488-conjugated anti-mouse secondary antibody (molecular Probe, Eugene, Oreg., USA) at 1:1000 dilutions for 1 hour at room temperature. F-actin was stained with Alexa488-conjugated Phalloidin at 1:100 dilutions for 1 hour at room temperature. Nuclei were counter-stained with 4′,6′ -diamidine-2′-phenylindole dihydrochloride (DAPI). Fluorescent images were obtained under a TCS SP2 AOBS microscope (Leica, Tokyo, Japan).

12.Bromodeoxyuridine Incorporation Assay

HEK293 cells transfected with plasmids designed to express LGN/GPSM2 or mock plasmids, were cultured in DMEM containing 10% FCS with 10 micro mol//L bromodeoxyuridine (BrdUrd). These cells were incubated for 24 hours and fixed; incorporated BrdUrd was measured using a commercially available kit (Cell Proliferation ELISA, BrdUrd; Roche Diagnostics, Basel, Switzerland) according to manufacturer's recommendation.

13.Immunoprecipitation

MCF7/Tet-OFF-HA-LGN/GPSM2 cells were incubated in doxycyclin-free medium for five days to induce the protein expression of HA-LGN/GPSM2. As a control, identical cells were maintained in doxycyclin-containing medium to suppress the expression. Then, the cells were synchronized with lmcg/ml aphidicolin for 16 h, followed by release for 10 h in aphidicolin-free medium to enrich the G2/M phase cells. Cells were lysed in immunoprecipitation buffer (50 mM Tris-HCl, 150 mM NaCl, 0.1% NP-40, 1 mM Na₃VO₄, 1 mM NaF, 0.1% Protease inhibitor Cocktail III, pH7.5), followed by incubation with anti-HA antibody agarose conjugate (Sigma). Bound proteins were eluted with HA-peptide, subjected to SDS-PAGE and stained with Silver stain DAIICHI (Daiichi Pure Chemicals, Tokyo, Japan). An approximately 55 kDa band, which was seen in immunoprecipitation products from HA-LGN/GPSM2 induced cells was extracted. Its peptide sequence was determined by MALDI-TOF mass spectrometry (Shimazu). For co-immunoprecipitation analysis, HEK293 cells were transfected with pCAGGSnHA-GPSM2, pCAGGSn3F-TRIOBP, pCAGGSn3F-PBK/TOPK or empty vector using FuGENE6 transfection regent (Roche). Transfected cells were lysed in immunoprecipitation buffer and immunoprecipitated using anti-HA agarose conjugate as described above. Bound proteins were eluted with HA-peptide and analyzed by SDS-PAGE and Western blotting.

14. Generation and Purification of His-Tagged Recombinant LGN/GPSM2

Escherichia coli strain BL21 codon-plus (DE3) RIL competent cells (Stratagene) was transformed with pET28a-LGN/GPSM2 and cultured in LB medium. Protein expression was induced by incubation with 0.5 mM isopropylbeta-D-thiogalactopyranoside (IPTG) at 25 degrees C. for 2 hours. Bacterial pellet was lysed in the lysis buffer (50 mM sodium-phosphate, 300 mM NaCl, 1% Tween20, 1 mM phenylmethylsulfonylfluoride, pH8.0) and followed by the affinity-purification using Ni-NTA superflow (QIAGEN) according to supplier's instruction.

15. Generation and Purification of GST-Tagged LGN/GPSM2

To generate GST-tagged LGN/GPSM2 recombinant protein, Escherichia coli strain BL21 codon-plus (DE3) RIL competent cells (Stratagene) was transformed with pGEX 6P-2-LGN/GPSM2 and cultured in LB medium. Protein expression was induced by incubation with 1.0 mM isopropyl-beta-D-thiogalactopyranoside (IPTG) at 27° C. for 2 hours. Bacterial pellet was lysed in the lysis buffer (40 mM Tris-HCl, 5 mM EDTA, 0.5% TritonX-100 supplemented with appropriate protease inhibitors, pH 8.0) and followed by the affinity-purification using Glutathione Sepharose™ 4B (GE Healthcare). GST-LGN/GPSM2 protein was bound to Glutathione Sepharose™ 4B at 4° C. for 1 hour, washed with lysis buffer for five times. Bound proteins were eluted with elution buffer (50 mM Tris-HCl, 150 mM NaCl, 2 mM DTT, 5% glycerol (v/v), 50 mM Glutathione (pH7.5)) subsequently dialyzed against dialyze buffer (50 mM Tris-HCl, 150 mM NaCl, 2 mM DTT, 5% glycerol (v/v) (pH7.5)).

16. In Vitro Kinase Assay

For PBK/TOPK assay, 0.4mcg of recombinat PBK/TOPK (Invitrogen) was incubated in 15 mcl kinase assay buffer (50 mM Tris-HCl, 10 mM MgCl₂, 2 mM dithio-threitol, 1 mM EGTA, 0.01% Brij-35, 100mcM ATP, pH7.5). For Aurora kinase assay, 0.05 mcg of recombinant Aurora kinase A or B (SignalChem) was incubated in 12 mcl Aurora-kinase assay buffer (25 mM MOPS, 12.5 mM beta-glycerol-phosphate, 25 mM MgCl2, 5 mM EGTA, 2 mM EDTA, 0.25 mM dithiothreitol and 100 mcM ATP, pH7.2). In both assays, samples were supplemented with SmcCi of [gamma-³²P]-ATP (Perkin Elmer). For substrate, 0.2 mcg of the full-length of LGN/GPSM2 recombinant protein was added into the reaction solutions. After incubation at 30 degrees C. for 60-120 min, the reactions were terminated by addition of SDS-sample buffer and analyzed by western blotting with anti-phosphorylated threonine antibody (Cell Signaling). Alternatively, as substrate, 1.0 mcg of GST-tagged LGN/GPSM2 recombinant proteins were added into the reaction solutions. After incubation at 30 degrees C. for 15-30 min, reactions were terminated by addition of SDS-sample buffer and subjected to autoradiography.

In-Gel Digestion, Mass Spectrometry

17. In-Gel Digestion, Mass Spectrometry

HA-LGN/GPSM2 was immunoprecipitated from MCF7 Tet-Off cells synchronized at mitosis using 0.3 mcg/ml Nocodazole for 18 hours as described above. Immunoprecipitated samples were subjected to SDS-PAGE followed by Coomassie-staining using SimplyBlue™ SafeStain (Invitrogen). The excised protein bands were reduced in 10 mM tris(2-carboxyethyl)phosphine (Sigma) with 50 mM ammonium bicarbonate (Sigma) for 30 min at 37 degrees C. and alkylated in 50 mM iodoacetamide (Sigma) with 50 mM ammonium bicarbonate for 45 min in the dark at 25 degrees C. Porcine trypsin (Promega) was added for a final enzyme to protein ratio of 1:20. The digestion was conducted at 37 degrees C. for 16 hours. Digests were analyzed in HCTultra-ETD II mass spectrometer (Bruker Daltonics) coupled to 1200 Series Rapid Resolution LC System (Agilent Technologies) with HPLC-Chip Cube (Agilent Technologies). The liquid chromatography separation was performed in Protein ID chip #2 (75 m 150 mm analytical column with 40 nl enrichment column) using a 35 min linear gradient from 5.4% to 29.2% of acetonitrile in 0.1% formic acid at 300 nl/min. MS/MS peak list was generated by Compass software (Bruker Daltonics) and exported to a local MASCOT search engine version 2.2.03 (Matrix Science) for protein data base search.

18. cDNA Mutagenesis

Site-directed mutagenesis was performed with two-step mutagenesis PCR. We generated the following mutations in pCAGGS-nHA-LGN/GPSM2 and pGEX6P-2-LGN/GPSM2: S401A, T519A and S558A. The oligonucleotides used to create point mutations in the LGN/GPSM2 cDNA were as follows: 5′-CGCCGGCAT GCTATGGAAAATATGG-3′ (SEQ ID NO:46) and 5′-CCATATTTTCCATAGC ATGCCGGCG-3′ (SEQ ID NO:47) for S401A, 5′-ACTTCTTCCGC ]TCCCC-CTAAAATG-3′ (SEQ ID NO:48) and 5′-CATTTTAGGGGGAGCGGAAGAAGT-3′ (SEQ ID NO:49) for T519A, 5′-CAGAGGGCTGCTTTCAGTAATTTG-3′ (SEQ ID NO:50) and 5′-CAAATTACTGAAAGCAGCCCTCTG-3′ (SEQ ID NO:51) for S558A (underlines indicate the nucleotides that were substituted from the wild type).

Thurough examples, positions of the amino acid residues of LGN/GPSM2 were shown according to the amino acid sequence defined by Genbank accession No. U54999 (SEQ ID NO: 53).

II. Results

1. Overexpression of LGN/GPSM2 in Breast Cancer Cells

The elevated expression of the LGN/GPSM2 was validated gene in 8 of 15 clinical breast cancer cases by semiquantitative RT-PCR analysis (FIG. 1A) as well as cDNA microarray data (Nishidate T et al., Int J Oncol. 2004 25:797-819). Subsequent northern-blot analysis using a LGN/GPSM2 cDNA fragment as a probe confirmed overexpression of an approximately 8-kb transcript of LGN/GPSM2 in breast cancer cell lines (FIG. 1B). On the other hand, LGN/GPSM2 expression was hardly detectable in any of vital organs (FIG. 1C) as concordant to the results of cDNA microarray analysis.

Since the assembled cDNA sequence of LGN/GPSM2 (FLJ20046 fis; AK000053.1; 1855 bp (SEQ ID NO: 38)) was much smaller than the 8-kb transcript indicated by northern-blot analysis, the inventers performed the exon-connection and 5′ RACE experiments, and obtained the partial cDNA sequence of LGN/GPSM2 consisting of 5611 nucleotides (Genebank accession; AB445462 (SEQ ID NO: 39)) containing the complete open reading frame sequence which encodes a protein of 684 amino acids (FIG. 1D; longer transcript). To validate the expression pattern of LGN/GPSM2, the present inventers did northern blot analysis using a probe where located in its coding region, and found overexpression of an approximately 4.0 kb transcript in breast cancer cell lines, indicating that this transcript is the splicing variant of LGN/GPSM2 gene (Genebank accession number NM_{—}013296 (SEQ ID NO: 41)) consisting of 3039 nucleotide. These two variants share same ORF sequences, and consist of 15 and 16 exons, respectively; the V2 variant lacked of exon 16.

2. Immunosytochemical-Staining Analysis of LGN/GPSM2

To characterize the biological role of LGN/GPSM2 protein in breast cancer cell, the present inventers first examined subcellular localization of endogenous LGN/GPSM2 by immunocytochemical-staining analysis using T47D cell. As shown in FIG. 2A, LGN/GPSM2 protein was weakly seen in nucleus and cytoplasm in interphase cells. After disappearance of nuclear membrane, LGN/GPSM2 gathered near chromosomes. From metaphase to anaphase, LGN/GPSM2 co-localized with microtubules at spindle pole (FIG. 2B). Then, LGN/GPSM2 was concentrated at midzone in the late anaphase cells and showed partial co-localization with microtubules and complete colocalization with F-actin at midbody of cytokinetic cells (FIG. 2C). It was confirmed that similar subcellular localizations of LGN/GPSM2 protein in MDA-MB-231 cells as well as T47D cells (data not shown).

3. Cell-Cycle Dependent Expression and Phosphorylation of LGN/GPSM2

Since LGN/GPSM2 was observed to be various localizations during mitosis, the cell-cycle dependent alteration of endogenous LGN/GPSM2 protein was investigated. The present inventers synchronized T47D cells at G1 phase with treatment of aphidicolin, and performed western blot and semi-quantitative RT-PCR analyses. The results showed that LGN/GPSM2 protein showed highest expression at G2/M phase (9-12h) at both transcriptional and protein levels (FIG. 3A and B). Then, expression of LGN/GPSM2 decreased immediately after entry of next G1 phase at protein as well as transcriptional levels. Furthermore, the inventers found that LGN/GPSM2 showed slow-migrating band-shift during G2/M phase, indicating its possible post-translational modification. To further investigate its expression during mitotic phase in more detail, T47D cells were synchronized with nocodazole and mitotic cells were harvested by gentle shaking-off. It was also confirmed that LGN/GPSM2 protein showed high expression and significant band-shift from 0 to 1.5 hours in synchronized mitotic cells (FIGS. 3C and D). To clarify this hypothesis, lamda protein phosphatase analysis was performed using nocodazole-treated T47D cell extracts (see Materials and methods), and it was found that its shift-band was disappeared after treatment of lamda protein phosphatase (FIG. 3E), indicating that phosphorylation of LGN/GPSM2 in mitotic cells.

4. Effect of LGN/GPSM2-siRNA on Growth of Breast Cancer Cell Lines

To investigate the role of LGN/GPSM2 in cell growth or survival, siRNA expression vectors specific to LGN/GPSM2 was constructed under the control of the U6 promoter (psiU6BX-siGPSM2, #1 and #2), and transfected them into T47D or BT20 cells, in which expression of LGN/GPSM2 was high level. Treatment of two siGPSM2 (si #1 and si #2) caused effectively reduction of LGN/GPSM2 exresion with control siRNAs (siEGFP and siSCR). MTT and colony formation assays revealed that the number of viable cells was reduced in both cell lines in comparison with controls (FIG. 4A-F). To confirm the specificity of siLGN/GPSM2, the expression vector encoding 3-base mismatch and scrambled sequence of siLGN/GPSM2-si #1 (si #1-mm and si #1-SCR) were constructed. T47D cells transfected with either mismatch or scramble show no growth suppression, showing the specificity of the siRNA sequence (FIG. 4G-I). To further investigate the effects of LGN/GPSM2 on the cell growth, the inventers did BrdUrd-incorporation assays using HEK293 cells transiently transfected with LGN/GPSM2-expressing plasmids. DNA synthesis seemed to be enhanced by the induction of LGN/GPSM2 expression (P=0.019) (FIGS. 5A and B). Further, the cell growth was examined using COS-7 cells transiently transfected with LGN/GPSM2-expressing plasmids. Cell-growth was significantly up-regulated by the induction of LGN/GPSM2 expression (p=0.004) (FIGS. 5C and D).

For detailed analysis, T47D cells was transfected with LGN/GPSM2-specific siRNA oligonucleotide (siLGN/GPSM2), and it was confirmed that the significant knockdown effect at the protein level (FIG. 6A). Knocking-down of LGN/GPSM2 expression resulted in a remarkable increase in the population of G1-phase cells (80.7%), compared to the cells transfected with a control siEGFP (71.6%) as shown by flow cytometry analysis (FIG. 6B). Morphological observation showed the intercellular bridge formation in the cells transfected with siLGN/GPSM2, suggesting the disordered cytokinesis (FIG. 6C). Thus, the apparent ‘G1-phase arrest’ of siLGN/GPSM2 was caused by such aberrantly divided cells tethered by intercellular bridge, which is highly fragile and torn off.

5. LGN/GPSM2 Interacts with TIOBP/Tara, F-Actin Associating Protein

It has been reported that LGN/GPSM2 is associated with several molecules, such as microtubule spindle-associatin protein, NuMA (Du Q et al., Curr Biol. 2002 12:1928-1933, Du Q et al., Nat Cell Biol. 2001 3:1069-1075, Du and Macara, Cell 2004 119:503-516) and heterotrimeric G-protein a subunit (G alpha; Mochizuki et al., Gene 1996 181:39-43, McCudden et al., Biochem Biophys Acta. 2005 1745:254-264). However, none of these proteins were sufficient to demonstrate the role of LGN/GPSM2 in cytokinesis of cancer cells. Therefore, to search its interacting protein(s), the present inventers immunoprecipitated LGN/GPSM2 from breast cancer cell line enriched in G2/M phase and isolated the interacting proteins by means of MS spectrometry analysis. This approach identified TRIOBP/Tara (Seipel et al., J Cell Sci. 2001 114:389-399) as a candidate protein (FIG. 7A). Co-immunoprecipitation assay confirmed the interaction of LGN/GPSM2 with TRIOBP/Tara. Interestingly, it was found that its interaction was enhanced in the mitotic cells which collected by treatment with nocodazole (FIG. 7B), indicating that these proteins interact in a cell-cycle dependent manner. Both endogenous LGN/GPSM2 and TRIOBP/Tara were observed to localize at midbody of dividing cells (FIG. 7C). By observations from cross-section, LGN/GPSM2 was found in the center of midbody as shown in FIG. 2C and abut with TRIOBP/Tara, which was seen in a continuous ring around the midbody (FIG. 7C, right two panel).

Since LGN/GPSM2 plays a role in the midbody of cytokinetic cells, expression and subcellular localization of F-actin in LGN/GPSM2-depleted cells was examined by siRNA treatment. Interestingly, the cytokinetic cells depleted of LGN/GPSM2 showed the poor F-actin structure between dividing cells compared to the cells treated with siEGFP (FIG. 7D), indicating that LGN/GPSM2 is indispensable for actin polymerization at midbody.

6. LGN/GPSM2 is Phosphorylated by PBK/TOPK at Mitosis

As described above, the transient phosphorylation of LGN/GPSM2 was found at G2/M phase in breast cancer cells. PBK/TOPK is a serine/threonine protein kinase and considered to be crucial in cytokinesis because it localizes at midbody at cytokinetic cells and knock down of its expression caused aberrant cell morphology with intercellular bridge due to cytokinetic failure (Park et al., unpublished data). FIG. 8A shows that LGN/GPSM2 interacted with PBK/TOPK and their interaction was enhanced in the mitotic cells. Phosphorylated PBK/TOPK seem to be preferentially associated with LGN/GPSM2. To investigate whether PBK/TOPK phosphorylates LGN/GPSM2 directly, the inventers performed kinase assay using a purified full-length LGN/GPSM2 recombinant protein and a full-length PBK/TOPK recombinant protein, and found phosphorylation of the LGN/GPSM2 protein by PBK/TOPK in vitro (FIG. 8B). Additionaly, kinase assay was performed using a purified full-length GST-LGN/GPSM2 recombinant protein and a full-lenght PBK/TOKP recombinant protein. The autoradiography images also showed phosphorylation of the LGN/GPSM2 protein by PBK/TOPK (FIG. 8C). Knocking-down of PBK/TOPK expression using siRNA oligonucleotide resulted in the disappearance of its phosphoryalted band in the cells synchronized at G2/M although the cell cycle was comparable to the cells treated with siEGFP (FIG. 8C), demonstrating the phosphorylation by PBK/TOPK in vivo.

7. Identification of S401, T519 and S558 of SEQ ID NO: 53 as Phosphorylation Sites on GPSM2

FIG. 3B, 3D and 3E indicated the existence of phosphorylated form of GPSM2 at mitosis. To explore the phosphorylation sites on GPSM2 in mitotic phase, transiently-expressed HA-tagged LGN/GPSM2 was immunoprecipitated from MCF-7 cells synchronized at mitosis and was analyzed with LC/MS/MS (FIG. 9A). Four LGN/GPSM2-derived peptides: residues 399-409, 508-526, and 551-566 were finally identified as phosphorylated peptides in the MASCOT database search (FIG. 9B-9D). This result indicated that LGN/GPSM2 was phosphorylated at serine 401, threonine 519 and serine 558 of SEQ ID NO: 53 phosphorylated in Nocodazole-treated MCF-7 cells. Threonine 519 and serine 558 are located in GoLoCo domain (FIG. 9E). Serine 401 is located in an amino acids sequence that is frequently found among the substrates of Aurora kinase family (Ohashi et al., 2006). Substitution of Threonine 519 to Ala showed the significant increase in mobility on SDS-PAGE (FIG. 9F), suggesting that significant change of mobility on SDS-PAGE was due to the phosphorylation at threonine 519. Substitution of serine 401 to alanine distinguished the phosphorylation of GST-LGN/GPSM2 by both Aurora kinase A and B, indicating LGN/GPSM2 is phosphorylated at serine 401 by Aurora kinase A and/or B at mitosis (FIG. 10A,10B). PBK/TOPK, which was has been shown to phosphorylate LGN/GPSM2 in FIG. 8, did not phosphorylate these amino acids identified here (FIG. 10C). Amino acids targeted by PBK/TOPK probably exist among the amino acids those were not covered by mass spectrometry analysis. Possible kinases for threonine 519 and serine 558 are still under investigation.

Overexpression of LGN/GPSM2 enhances the cell growth as shown in FIG. 5B. To determine whether phosphorylation at serine 401, threonine 519 and/or serine 558 of SEQ ID NO: 53 are required for LGN/GPSM2-mediated growth enhancement, MTT assay was performed with COS-7 cells transfected with wild type or each substitutes. While wild type LGN/GPSM2 enhanced cell growth compared with mock transfectant, all substitutes suppressed growth-enhancement activity to mock control level (FIG. 11), suggesting that all of these phosphorylation sites contribute to growth control. Whether these phosphorylation sites regulate the cell growth by different or same mechanism is to be elucidated.

III. Discussion

Through identification and characterization of cancer-related genes and their products, molecular-targeting drugs for cancer therapy have been developed in the last two decades, but the proportion of patients who are able to have a benefit by presently-available treatments is still very limited (Navolanic and McCubrey Int J Oncol. 2005 27:1341-1344, Bange et al., Nat Med. 2001 7:548-552). Therefore, it is urgent to develop new anticancer agents that will be highly specific to malignant cells and have the minimal risk of adverse reactions. In the present invention, LGN/GPSM2 has been demonstrated to be upregulated in clinical breast cancer cases and cell lines, but to be hardly detectable in any normal human vital tissues examined.

LGN/GPSM2 gene encodes a putative 684-amino acid protein that contains six highly-conserved TPR (Tetratricopeptide repeats) domains at N-termius and four GoLoco domains at C-terminus which were predicted by SMART prediction. These results also demonstrated that LGN/GPSM2 protein was mainly localized in the

WO 2010/023854 PCT/JP2009/004017 nucleus of interphase cells, accumulated as a series of narrow bars at spindle midzone in the anaphase cells, and was finally concentrated at the contractile ring in telophase and cytokinesis stages. These findings demonstrate a role of this protein in cell-cycle progression.

It has been further demonstrated by means of the siRNA technique that knocking down of the endogenous LGN/GPSM2 expression significantly suppressed the cell growth of breast cancer cell lines, T47D and BT-20, due to abnormal cell division and subsequent cell death, probably due to the dysfunction in the cytokinetic process.

These findings imply important roles of LGN/GPSM2 in growth of breast cancer cells and demonstrate that LGN/GPSM2 is a molecular target for the treatment of breast cancer.

### INDUSTRRIAL APPLICABILITY

The gene-expression analysis of cancers described herein using the combination of laser-capture dissection and genome-wide cDNA microarray has identified specific genes as targets for cancer prevention and therapy. Based on the expression of a differentially expressed gene, LGN/GPSM2, the present invention provides a molecular dia gnostic marker for identifying and detecting cancer, in particular, breast cancer.

The data provided herein add to a comprehensive understanding of cancers, facilitate development of novel diagnostic strategies, and facilitate identification of molecular targets for therapeutic drugs and preventative agents. Such information contributes to a more profound understanding of tumorigenesis, and provide indicators for developing novel strategies for diagnosis, treatment, and ultimately prevention of cancers.

All patents, patent applications, and publications cited herein are incorporated by reference in their entirety.

Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method for diagnosing cancer or a predisposition for developing cancer in a subject, comprising the step of determining the expression level of the LGN/GPSM2 gene in a subject-derived biological sample, wherein an increase in said expression level as compared to a normal control level of said gene indicates that said subject suffers from or is at a risk of developing cancer.

2. The method of claim 1, wherein said expression level is at least 10% greater than the normal control level.

3. The method of claim 1, wherein said expression level is determined by any of the methods selected from the group consisting of:
(a) detecting mRNA of the LGN/GPSM2 gene;
(b) detecting a protein encoded by the LGN/GPSM2 gene; and
(c) detecting a biological activity of the protein encoded by the LGN/GPSM2 gene.

4. The method of claim 1, wherein the subject-derived biological sample is biopsy.

5. The method of claiml, wherein the cancer is breast cancer.

6. A kit for diagnosing or detecting cancer, wherein said kit comprises a detection reagent which binds to the transcription or translation product of the LGN/GPSM2 gene.

7. The kit of claim 6, wherein the cancer is breast cancer.

8. A method of screening for a candidate compound for treating or preventing cancer, which comprises the steps of:
(a) contacting a test compound with the LGN/GPSM2 polypeptide or a fragment thereof;
(b) detecting the binding between the polypeptide or fragment and the test compound; and
(c) selecting the test compound that binds to the polypeptide or fragment as a candidate compound for treating or preventing cancer.

9. A method of screening for a candidate compound for treating or preventing cancer, wherein said method comprises the steps of:
(a) contacting a test compound with the LGN/GPSM2 polypeptide or a fragment thereof;
(b) detecting the biological activity of the polypeptide or fragment;
(c) comparing the biological activity of the polypeptide or fragment with the biological activity detected in the absence of the test compound; and
(d) selecting the test compound that suppresses the biological activity of the polypeptide as a candidate compound for treating or preventing cancer.

10. The method of claim 9, wherein the biological activity is cell proliferative activity or DNA synthesis enhancing activity.

11. A method of screening for a candidate compound for treating or preventing cancer, which comprises the steps of:
(a) contacting a test compound with a cell expressing the LGN/GPSM2 gene;
(b) detecting the expression level of the LGN/GPSM2 gene;
(c) comparing the expression level with the expression level detected in the absence of the test compound; and
(d) selecting the test compound that reduces the expression level as a candidate compound for treating or preventing cancer.

12. A method of screening for a candidate compound for treating or preventing cancer, wherein said method comprises the steps of:
(a) contacting a test compound with a cell introduced with a vector that comprises the transcriptional regulatory region of the LGN/GPSM2 gene and a reporter gene expressed under the control of the transcriptional regulatory region;
(b) measuring the expression level or activity of said reporter gene;
(c) comparing the expression level or activity with the expression level or activity detected in the absence of the test compound; and
(d) selecting the test compound that reduces the expression level or activity as a candidate compound for treating or preventing cancer.

13. A method of screening for a candidate compound for treating or preventing cancer, said method comprising the steps of:
(a) contacting a polypeptide comprising a TRIOBP/tara-binding domain of a LGN/GPSM2 polypeptide with a polypeptide comprising a LGN/GPSM2-binding domain of a TRIOBP/tara polypeptide in the presence of a test compound;
(b) detecting binding between the polypeptides; and
(c) selecting the test compound that inhibits the binding between the polypeptides as a candidate compound for treating or preventing cancer.

14. The method of claim 13, wherein the polypeptide comprising the TRIOBP/tara-binding domain comprises a LGN/GPSM2 polypeptide.

15. The method of claim 13, wherein the polypeptide comprising the LGN/GPSM2-binding domain comprises a TRIOBP/tara polypeptide.

16. A method of screening for a candidate compound for treating or preventing cancer, said method comprising the steps of:
(a) contacting a polypeptide comprising a PBK/TOPK-binding domain of a LGN/GPSM2 polypeptide with a polypeptide comprising a LGN/GPSM2-binding domain of a PBK/TOPK polypeptide in the presence of a test compound;
(b) detecting binding between the polypeptides or the phosphorylation level of the polypeptide comprising a PBK/TOPK-binding domain of a LGN/GPSM2 polypeptide; and
(c) selecting the test compound that inhibits binding between the polypeptides or the phosphorylation level of LGN/GPSM2 as a candidate compound for treating or preventing cancer.

17. The method of claim 16, wherein the polypeptide comprising the PBK/TOPK-binding domain comprises a LGN/GPSM2 polypeptide.

18. The method of claim 16, wherein the polypeptide comprising the LGN/GPSM2-binding domain comprises a PBK/TOPK polypeptide.

19. A method of screening for a candidate compound for treating or preventing cancer, said method comprising the steps of:
(a) contacting a LGN/GPSM2 polypeptide or a functional equivalent thereof with a protein kinase in the presence of a test compound under a suitable condition for phosphorylation;
(b) detecting the phosphorylation level of the LGN/GPSM2 polypeptide or functional equivalent thereof at one or two serine residues and/or a threonine residue corresponding to Ser401, Thr519 and/or Ser558 in the amino acid sequence of SEQ ID NO: 53;
(c) comparing the phosphorylation level with the expression level or activity detected in the absence of the test compound; and
(d) selecting the test compound that reduces the phosphorylation level as a candidate compound for treating or preventing cancer.

20. The method of claim 8, wherein the cancer is breast cancer.

21. A double-stranded molecule comprising a sense strand and an antisense strand, wherein the sense strand comprises a nucleotide sequence corresponding to a target sequence consisting of SEQ ID NO: 20 or 21, and wherein the antisense strand comprises a nucleotide sequence which is complementary to said sense strand, and wherein said double-stranded molecule, when introduced into a cell expressing the LGN/GPSM2 gene, inhibits expression of said gene.

22. The double-stranded molecule of claim 21, wherein the sense strand hybridize with antisense strand at the target sequence to form the double-stranded molecule having between 19 and 25 nucleotide pair in length.

23. The double-stranded molecule of claim 21, wherein said double-stranded molecule is a single oligonucleotide comprising the sense strand and the antisense strand linked via a single-stranded nucleotide sequence.

24. The double-stranded molecule of claim 21, wherein said polynucleotide has the general formula 5′-[A]-[B]-[A′]-3′ wherein [A] is a nucleotide sequence comprising SEQ ID NO: 20 or 21; [B] is a nucleotide sequence consisting of about 3 to about 23 nucleotides; and [A′] is a nucleotide sequence complementary to [A].

25. A vector comprising each or both of a combination of polynucleotide comprising a sense strand nucleic acid and an antisense strand nucleic acid, wherein said sense strand nucleic acid comprises nucleotide sequence of SEQ ID NOs: 20 or 21, and wherein the antisense strand comprises a nucleotide sequence which is complementary to said sense strand, wherein the transcripts of said sense strand and said antisense strand hybridize to each other to form said double-stranded molecule, and wherein said vector, when introduced into a cell expressing the LGN/GPSM2 gene, inhibits expression of said gene.

26. (canceled)

27. The vector of claim 25, wherein the polynucleotide is an oligonucleotide of between about 19 and 25 nucleotides in length.

28. The vector of claim 25, wherein said double-stranded molecule is a single nucleotide transcript comprising the sense strand and the antisense strand linked via a single-stranded nucleotide sequence.

29. The vector of claim 28, wherein said polynucleotide has the general formula 5′-[A]-[B]-[A′]-3′, wherein [A] is a nucleotide sequence comprising SEQ ID NO: 20 or 21; [B] is a nucleotide sequence consisting of about 3 to about 23 nucleotide; and [A′] is a nucleotide sequence complementary to [A].

30. A method of treating or preventing cancer expressing LGN/GPSM2 in a subject comprising administering to said subject a pharmaceutically effective amount of a double-stranded molecule against a LGN/GPSM2 gene or a vector encoding thereof, wherein said double-stranded molecule inhibits the cell proliferation contacting with the cell expressing LGN/GPSM2 gene as well as the expression of the LGN/GPSM2 gene, and a pharmaceutically acceptable carrier.

31. A method of claim 30, wherein the double-stranded molecule comprises a sense strand and an antisense strand, wherein the sense strand comprises a nucleotide sequence corresponding to a target sequence consisting of SEQ ID NO: 20 or 21, and wherein the antisense strand comprises a nucleotide sequence which is complementary to said sense strand, and wherein said double-stranded molecule, when introduced into a cell expressing the LGN/GPSM2 gene, inhibits expression of said gene, wherein the vector comprises each or both of a combination of polynucleotide comprising a sense strand nucleic acid and an antisense strand nucleic acid, wherein said sense strand nucleic acid comprises nucleotide sequence of SEQ ID NOs: 20 or 21, and wherein the antisense strand comprises a nucleotide sequence which is complementary to said sense strand, wherein the transcripts of said sense strand and said antisense strand hybridize to each other to form said double-stranded molecule, and wherein said vector, when introduced into a cell expressing the LGN/GPSM2 gene, inhibits expression of said gene.

32. A method of claim 30, wherein the cancer expressing LGN/GPSM2 is breast cancer or hepatocellular carcinoma.

33. A composition for treating or preventing cancer, which comprises a pharmaceutically effective amount of a double-stranded molecule against a LGN/GPSM2 gene or a vector encoding thereof, wherein the double-stranded molecule inhibits the cell proliferation contacting with the cell expressing LGN/GPSM2 gene as well as the expression of the LGN/GPSM2 gene, and a pharmaceutically acceptable carrier.

34. A composition of claim 33, wherein the double stranded molecule comprises a sense strand and an antisense strand, wherein the sense strand comprises a nucleotide sequence corresponding to a target sequence consisting of SEQ ID NO: 20 or 21, and wherein the antisense strand comprises a nucleotide sequence which is complementary to said sense strand, and wherein said double-stranded molecule, when introduced into a cell expressing the LGN/GPSM2 gene, inhibits expression of said gene, wherein the vector comprises each or both of a combination of polynucleotide comprising a sense strand nucleic acid and an antisense strand nucleic acid, wherein said sense strand nucleic acid comprises nucleotide sequence of SEQ ID NOs: 20 or 21, and wherein the antisense strand comprises a nucleotide sequence which is complementary to said sense strand, wherein the transcripts of said sense strand and said antisense strand hybridize to each other to form said double-stranded molecule, and wherein said vector, when introduced into a cell expressing the LGN/GPSM2 gene, inhibits expression of said gene.

35. The composition of claim 33, wherein the cancer is breast cancer.

36. An isolated nucleic acid comprising the nucleotide sequence of SEQ ID NO: 39.

37. The method of claim 9, wherein the cancer is breast cancer.

38. The method of claim 11, wherein the cancer is breast cancer.

39. The method of claim 12, wherein the cancer is breast cancer.

40. The method of claim 13, wherein the cancer is breast cancer.

41. The method of claim 16, wherein the cancer is breast cancer.

42. The method of claim 19, wherein the cancer is breast cancer.
